(19) United States
(12) Patent Application Publication
(10) Pub. No.: US 2013/0315886 A1
(43) Pub. Date: Nov. 28, 2013

(54) RETROELEMENTS AND MENTAL DISORDERS AND METHODS OF MEASURING L1 RETROTRANSPOSITION

(71) Applicants: Fred H. Gage, La Jolla, CA (US); Nicole Coufal, San Diego, CA (US); Michael J. McConnell, Charlottesville, VA (US); Alysson Muotri, La Jolla, CA (US); Maria C.N. Marchetto, La Jolla, CA (US)

(72) Inventors: Fred H. Gage, La Jolla, CA (US); Nicole Coufal, San Diego, CA (US); Michael J. McConnell, Charlottesville, VA (US); Alysson Muotri, La Jolla, CA (US); Maria C.N. Marchetto, La Jolla, CA (US)

(73) Assignee: Salk Institute for Biological Studies, La Jolla, CA (US)

(21) Appl. No.: 13/883,962

(22) PCT Filed: Aug. 4, 2010
(86) PCT No.: PCT/US2010/044358

§ 371 Date: Aug. 8, 2013

Related U.S. Application Data
(60) Provisional application No. 61/273,599, filed on Aug. 5, 2009.
(60) Provisional application No. 61/231,663, filed on Aug. 5, 2009.

(51) Int. Cl. C12Q 1/68

(52) U.S. Cl. CPC C12Q 1/6883
USPC 424/94.1; 506/9; 435/375; 435/368; 435/6.11

(57) ABSTRACT

A method of treating increased non-LTR retrotransposition in a cell. The method includes exposing a neural cell to a retrotransposition inhibitor in an amount sufficient to decrease the non-LTR retrotransposition in the neural cell or a progeny of the neural cell. In various embodiments, the non-LTR retrotransposition involves at least one L1 retrotransposon. Also provided is a method of assaying retrotransposition in neural cells. The method includes sorting synchronized neural cells of the same genetic background into single neural cells, and subjecting one or more of the sorted single neural cells to quantitative polymerase chain reaction amplification of at least one retrotransposon. In addition, a method of identifying an inhibitor of retrotransposition and a identifying a neural condition associated with non-LTR retrotransposition are provided.

### CROSS-REFERENCE TO RELATED APPLICATIONS

This application claims the benefit of Provisional Patent Application No. 61/231,663, filed on Aug. 5, 2009, and Provisional Patent Application No. 61/273,599, filed on Aug. 5, 2009, which are both incorporated by reference herein.

### STATEMENT REGARDING FEDERALLY SPONSORED RESEARCH OR DEVELOPMENT

This invention was made with Government support under Grant No. R56 MH082070 from the National Institute of Health. The Government has certain rights in this invention.

### REFERENCE TO SEQUENCE LISTING

This application contains a paper copy and an electronic form of a sequence listing. The contents of sequence listing are incorporated herein by reference.

### BACKGROUND

1. Field of the Invention

The invention relates to retrotransposons in neural cells.

2. Related Art

L1s are abundant retrotransposons that comprise approximately 20% of mammalian genomes (1-3). Recently-evolved L1s are polymorphic, resulting in individual variations in retrotransposition capacity (4,5). Although most L1s are retrotransposition-defective (6,7), active L1 retrotransposons can impact the genome in a variety of ways, creating insertions, deletions, new splice sites or gene expression fine-tuning (8-10). Previous data showed that, during neuronal differentiation, an EGFP-tagged L1 element could insert near or within neuron-associated genes, affecting gene expression and cell function. An analysis of the sequence data from several L1 insertions in neuronal precursor cells (NPCs), derived from neural stem cells (NSCs), indicated that the integration process might be regulated (11). Thus, neuronal networks may be affected by de novo L1 insertions during brain development (12,13).

### BRIEF SUMMARY

In one aspect, a method of treating non-LTR retrotransposition that occurs in neural cells is provided. The method includes exposing a neural cell to a retrotransposition inhibitor in an amount sufficient to decrease non-LTR retrotransposition occurring in the neural cell or a progeny of the neural cell. The neural cell can be a neural stem cell or a neural precursor cell. In particular embodiments, the neural cell is a mammalian cell such as a human cell, and can be a fetal or embryonic cell. The neural cell can be identified with a nervous system condition that results from non-LTR retrotransposition in neural cells. In some embodiments, the neural cell is in a patient, which can be a newborn, a child or an adult. In some embodiments, the neural cell is in an embryo or fetus in a pregnant patient. The non-LTR retrotransposition can involve at least one L1 retrotransposon. A decrease in non-LTR retrotransposition can be determined by comparison to a control cell, for example, by comparison to a comparable neural cell not exposed to a retrotransposition inhibitor.

Nervous system conditions resulting from non-LTR retrotransposition in neural cells include, but are not limited to, autism or autism spectrum disorders, schizophrenia, Rett syndrome, Tourette syndrome, ataxia telangiectasia and other ataxias, xeroderma pigmentosum, Cockyne syndrome, fragile x, aspergers syndrome, childhood disintegrative disorder, tuberous sclerosis complex, or psychiatric disorders such as neurogiromatosis, Prader-Willi, Angelman, Joubert, Down, Williams or Cowdern syndrome or other psychiatric disorders, or any combination of conditions thereof.

Transposition inhibitors include, but are not limited to, anti-retroviral drugs, inhibitors of RNA stability, inhibitors of reverse transcription, inhibitors of L1 endonuclease activity, stimulators of DNA repair machinery, zinc-fingers that target the L1 promoter region, enzymes that inhibit L1, repressors that inhibit L1, or any combination thereof.

In another aspect, a method of assaying retrotransposition in neural cells is provided. The method includes sorting synchronized neural cells of the same genetic background into single neural cells, and subjecting one or more of the sorted single neural cells to quantitative polymerase chain reaction (“qPCR”) amplification of at least one retrotransposon. The synchronized neural cells can be neural stem cells or neural precursor cells. In some embodiments, the content of the at least one retrotransposon determined by the qPCR amplification is compared to the content of the at least one retrotransposon in one or more control cells. The control cells can be neural or non-neural cells depending on the type of comparison, and are of the same, or comparable, genetic background as the synchronized neural cells. In various embodiments, the retrotransposon is a non-LTR retrotransposon, and can be an L1 retrotransposon.

In a further aspect, a method of identifying an inhibitor of retrotransposition is provided. The method includes exposing one or more neural precursor cells to a candidate inhibitor, determining the content of at least one retrotransposon in the one or more neural precursor cells or in progeny of the one or more neural precursor cells, or both, and comparing the content of the at least one retrotransposon in the one or more neural precursor cells, or their progeny, or both, to the content of the at least one retrotransposon in one or more control cells not exposed to the candidate inhibitor. Depending on the type of comparison, the control cells can be neural precursor cells, or their progeny, or both. In this method, a decrease in the content of the retrotransposon in the one or more neural precursor cells, or their progeny, or both, compared to the one or more control cells is indicative of inhibition of retrotransposition. In various embodiments, the retrotransposon is a non-LTR retrotransposon, and can be an L1 retrotransposon.

In another aspect, a method of identifying a neural condition associated with non-LTR retrotransposition is provided. The method includes determining the content of at least one non-LTR retrotransposon in a neural cell in comparison to the content of the at least one non-LTR retrotransposon in one or more control cells. In this method, the neural cell is of a genotype associated with a nervous system condition. An increase in non-LTR retrotransposition content in neural cells versus control cells is an indication that the nervous system condition is associated with non-LTR retrotransposition. The nervous system condition can be autism or autism spectrum disorders, schizophrenia, Rett syndrome, Tourette syndrome, ataxia telangiectasia and other ataxias, xeroderma pigmentosum, Cockyne syndrome, fragile x, aspergers syndrome, childhood disintegrative disorder, tuberous sclerosis complex, or neurogiromatosis, Prader-Willi, Angelman, Joubert, Down, Williams and Cowdern syndrome or other psychiatric disorders, or any combination of conditions thereof. In some embodiments, the neural cell is from a knockout animal or an individual having the nervous system condition. In various embodiments, the retrotransposon is a non-LTR retrotransposon, and can be an L1 retrotransposon.

In a further aspect, highly efficient methods to measure Line-1 retrotransposition in tissue samples and single cells are provided. The methods and procedures provided herein may be used to measure Line-1 retrotransposition in a single cell as well as in multi-cellular samples. The assay may be used to monitor Line-1 retrotransposition in individual cells derived from fresh or frozen tissue samples, biopsies, fertilized eggs, induced pluripotent stem cells as well as tumor cells and therefore provides a valuable tool to monitor genomic mosaicism and genomic rearrangement. The present invention provides a novel diagnostic tool to monitor genomic rearrangement in cells. Examples for areas of application are cancer diagnosis, genomic screening in the context of in vitro fertilization, preventative screening, diagnosis of neurologic disorders as well as measuring neural plasticity.

In another aspect, a method of measuring Line-1 retrotransposition activity in single cells is provided. The method includes separating a tissue into single cells and isolating genomic DNA from the single cells, thereby forming single cell DNA samples. The single cell DNA samples are incubated with Line-1 primers and control primers. A Line-1 DNA is amplified with the Line-1 primers, thereby forming an amplified Line-1 DNA and a control DNA is amplified with the control primers, thereby forming an amplified control DNA. An amount of the amplified Line-1 DNA is compared with an amount of the amplified control DNA, thereby measuring Line-1 retrotransposition activity in the single cells.

In another aspect, a kit for measuring Line-1 retrotransposition activity in a single cell is provided. The kit includes Line-1 specific primers, control primers and a single cell.

In a further aspect, a kit for measuring Line-1 retrotransposition activity in a tissue is provided. The kit includes Line-1 specific primers, control primers and a tissue.

### BRIEF DESCRIPTION OF THE DRAWINGS

For a more complete understanding of the present invention, reference is now made to the following descriptions taken in conjunction with the accompanying drawings.

FIG. 1 is a panel showing that MeCP2 silences L1 expression by a repressor association with the L1 promoter region. (A) In vitro methylation by the Hpa II enzyme reduced the transcriptional activity up to 50% of the 5′UTR promoter. (B) Transfection of NSCs with siRNAs against MeCP2 mRNA reduced protein levels by approximately 65%. (C) SiRNA inactivation of MeCP2 transcripts correlates with increased L1 promoter activity. NSCs were transfected with a luciferase reporter gene driven by a wild-type (WT) L1 promoter (control=siRNA scramble) or siRNA against the MeCP2 mRNA. (D) Co-immunoprecipitation between MeCP2 and Sox2. Using high stringency conditions, MeCP2 IP resulted in a Sox2-enriched signal by Western blot. The reverse conditions provided similar results; Sox2 IP resulted in a MeCP2 enriched signal. Two different exposure times (20″) and (60″) are displayed. (E) L1 promoter activity, measured by luciferase expression, was higher in neuroepithelial cells that lack MeCP2 when compared to WT cells. Sox2 overexpression reduced the luciferase expression by approximately 50% in MeCP2 KO cells. Control cells, co-transfected with the pGL3-basic plasmid, gave no detectable signals (not shown). (F) Dynamic recruitment of MeCP2 and Sox2 on the endogenous rat L1 promoter region by ChIP. Extracts of formaldehyde-fixed cells were precipitated with MeCP2 antibody either in undifferentiated state (FGF-2) or after induction to neuronal differentiation (RA/FSK) and then analyzed by PCR with primers for the L1 5′UTR. (G) Occupancy of MeCP2 on the endogenous rat L1 promoter region requires DNA methylation. Removal of DNA methylation by treatment with 5-Azacitidine (5-Aza) reduced MeCP2 but not Sox2 association to the L1 promoter region. (H) Co-immunoprecipitation between MeCP2 and Sox2 in the presence or not of 5-Aza treatment. The association of MeCP2 and Sox2″ increased in the presence of 5-Aza. Error bars in all panels show s.e.m.

FIG. 2 is a panel showing that MeCP2 modulates neuronal L1 retrotransposition in vivo. (A) Analysis of L1-EGFP retrotransposition in the brains of WT and MeCP2 KO animals. EGFP-positive cells, indicating de novo somatic L1 retrotransposition, were found in several regions of the brain, but new insertions were increased in numbers in certain areas in the MeCP2 KO genetic background, such as the cerebellum and striatum. (B) Double-blinded quantification of whole brain sections in MeCP2 KO background revealed overall more EGFP-positive cells when compared to WT (6 age-matched animals were analyzed per group). Error bars in all panels show s.e.m. (C) Non-random distribution of L1 retrotransposition events in mouse brain. Representative images from a three-dimensional reconstruction of WT and MeCP2 KO mouse brains carrying the L1-EGFP transgene. Single dots (green) represent individual neurons that supported L1-EGFP retrotransposition. Different brain regions (olfactory bulb in red, striatum in magenta and cerebellum in cyan) are highlighted for better comparison. The increased number of EGFP-positive neurons, compared to the WT brain in each structure, is indicated below the MeCP2 KO model. R, rostral; C, caudal; D, dorsal and V, ventral.

FIG. 3 is a panel showing detection of L1 retrotransposition in other tissues of the L1-EGFP transgenic mice in the MeCP2 KO genetic background. Adult transgenic animals carrying only the L1-EGFP transgene were selected for tissue analysis. Several tissue samples were prepared as described for the brain (see Methods in Examples) and analyzed by immunofluorescence after staining with anti-GFP antibody. EGFP-positive cells were only found in testes (inset, 40×) but not in several other somatic cells tested, such as skin, muscle, liver, lung, heart or intestine. Bar=250 μm.

FIG. 4 is a panel showing endogenous L1 retrotransposition in neuroepithelial cells. (A) Neuroepithelial cells were harvested from E11.5 sibling embryos. Synchronized cells were sorted in individual wells of 96-well plates followed by qPCR. (B) The graph represents the distribution of CT values (inversely correlated to L1 amount) as a function (%) of its frequency in the cell population (each experiment used a population of 96 cells). Approximately 50% of cells in the MeCP2 KO background had higher L1 ORF2 DNA content compared to WT cells. (C) Fibroblasts derived from the different genetic backgrounds did not display variation in the L1 ORF2 DNA amount and were more homogeneous compared to neuroepithelial cells. CT values cannot be compared between neuroepithelial cells and fibroblasts due to differences in culture conditions. (D) Internal controls using specific primers for the L1 5′UTR and 5S RNA ribosomal genes (E) in neuroepithelial cells. None showed significant differences in DNA content between WT and MeCP2 KO genetic backgrounds. “Water” in all graphs represents a pool of CT values obtained from several independent experiments.

FIG. 5 is a panel showing multiplex qPCR for L1 sequences in human tissues. (A) Detecting genetic variation of L1 sequences in somatic tissues. Samples from brain and heart from the same individuals were obtained from Rett syndrome (RTT) patients and age/gender-matched controls. After DNA extraction, a Taqman multiplex qPCR approach was used to compare the number of L1 ORF2 sequences in the human genome. Primers for L1 ORF2 were used to multiplex with primers for control sequences. (B) The 5S ribosomal RNA gene (5S) is a non-mobile, conserved and repetitive control sequence. The inverse ratio of ORF2/5S represents the amount of L1 ORF2 DNA sequence in each sample. Under these conditions, L1 ORF2 sequences are more frequent in brains when compared to heart tissue from five individuals. Moreover, RTT patients' brains show significantly more L1 ORF2 sequences when compared to control individuals. Similar results were obtained when different primers/probe for ORF2 (ORF2-2, see Methods) were multiplex/normalized to other control sequences, such as the L1 5′UTR (C), using two different pair of primers (5′UTR-1 or 5′UTR-2); the non-mobile human endogenous retrovirus-H sequences (HERV) multiplexing with primers/probes ORF2-1 (D) or tandem copies of the satellite alpha (SATA) sequences multiplexing with primers/probe ORF2-2 (E). These graphics were derived by grouping different individuals, represented in FIG. 21. Error bars in all panels show s.e.m.

FIG. 6 is a panel showing MeCP2 protein amount and L1 5′UTR DNA methylation status during neuronal differentiation. (A) MeCP2 protein levels remains constant during a 4-day neuronal differentiation protocol. (B) Five CpG islands, close to the transcription starting point of L1s, were analyzed by bisulfite. 5-Azacytidine treatment for 4-days completely removed methyl radicals from the 5′UTR L1 region under analysis. Open circles=unmethylated CpG site, closed circles=methylated CpG site, dashes=L1 polymorphisms when compared to the template sequence. (C) Analysis of each of the 5 CpG sites analyzed in the L1 5′UTR promoter region revealed tendency of CpG islands in the 5′UTR L1 promoter region to de-methylated during neuronal differentiation, specially at the 3′ end, close to the transcriptional starting point.

FIG. 7 is a drawing of an L1-EGFP retrotransposition reporter strategy. The L1-EGFP transgenic mouse harbors a retrotransposition-competent human L1 element under the control of its endogenous promoter and carries an EGFP reporter construct in its 3′ UTR region. The EGFP gene is interrupted by the γ-globin IVS2 intron in the same transcriptional orientation as the L1 transcript. This arrangement ensures that EGFP-positive cells will arise only when a transcript initiated from the promoter driving L1 expression is spliced, reverse transcribed, and integrated into chromosomal DNA, thereby allowing expression of the EGFP gene from the pCMV promoter. In our animal model, the retrotransposed EGFP gene was detected in neurons and in germ cells, but not in other somatic tissues analyzed (11).

FIG. 8 is a panel showing L1-EGFP retrotransposition in different brain regions. EGFP-positive cells can be found in several anatomical regions of the brain. In the MeCP2 KO background, some regions, such as the striatum and cerebellum are more prone to retrotransposition than others, such as the cortex. The images illustrate the reproducibility of that susceptibility in two different animals from the two genetic backgrounds (WT animals ID#3 and 5; MeCP2 KO animals ID #8 and 12).

FIG. 9 is a panel showing measurement of endogenous L1 retrotransposition in neuroepithelial cells. (A) In vitro cell duplication timing of WT and MeCP2 KO neuroepithelial cells. (B) Representative image of a metaphase chromosome spread from a single mouse neuroepithelial cell with the expected 40 chromosomes. All cell preparations were karyotyped using standard protocols for chromosome spread preparation in metaphase and counterstaining with DAPI (4′,6-diamidino-2-phenylindole). At this stage of neural development, both genetic backgrounds showed a majority of neuroepithelial cells (˜95%) with 40 chromosomes. (C) Single-cell amplification using primers for L1 ORF2 region. Each lane represents the amplification product of the qPCR reaction in wells containing single cells or water as a negative control. All products have the expected length of about 50 bp and the sequences aligned to endogenous potential active mouse L1 elements. (D) Linear amplification by genomic qPCR. Single fibroblast cells from WT mice were mixed with known copies of L1 plasmid template prior to the reaction.

FIG. 10 is a graph showing higher number of L1 ORF2 sequences in MeCP2 KO neuroepithelial cells. The variation in the number of L1 ORF2 sequences, measured by qPCR, in fibroblasts derived from WT and MeCP2 KO background is around 10%. In contrast, neuroepithelial cells from the MeCP2 KO background can have up to 22.8% more L1 ORF2 sequences when compared to the WT mean.

FIG. 11 is a graph of L1 number copy quantification in the brains of RTT patients. Genomic DNA from RTT and control brains (80 pg), as well as, control DNA mixed with 100, 1000 and 10000 copies of L1 plasmid template. Given that the genome of a single cell is approximately 6.6 pg of genomic DNA, each reaction represents DNA from approximately 12 cells. Therefore, the copy number increase in RTT brains compared to controls is an average of 10 L1 insertions/cell.

FIG. 12 is a panel of graphs showing genetic variation of L1 sequences in somatic tissues. L1 ORF2 content in brain and heart samples from different individuals was obtained by multiplex qPCR using different primers and probes for ORF2 and normalized by 5S RNA ribosomal gene (A), L1 5′UTR (B), satellite alpha (SATA) (C) or human endogenous retrovirus-H (HERV) (E) sequences. Individual ID numbers are shown on the “x” axis. RTT patients and controls displayed a higher variability in ORF2 content in brain compared to a more homogeneous distribution in heart tissue. These data originated FIG. 5. Error bars in all panels show s.e.m.

FIG. 13 is a panel showing increased L1 retrotransposition in neuronal precursor cells (NPCs) derived from induced pluripotent cells iPSCs-RTT. (A) Schematic view of the NPC differentiation protocol from iPSCs, followed by L1_{RE3}-EGFP electroporation. (B) Representative images of NPCs expressing EGFP after de novo L1 retrotransposition. Bar=10 μm. (C) Quantification of the EGFP-positive cells in NPCs 7 days after transfection. Error bars in all panels show s.e.m. (D) PCR analysis of genomic DNA isolated from different NPC populations transfected with the L1_{RE3}-EGFP plasmid. The 1,243 bp PCR product corresponds to the original L1 vector harboring the intron-containing EGFP indicator cassette. The 343-bp PCR product, diagnostic for the loss of the intron, indicates a retrotransposition event.

FIG. 14 is a panel showing L1 retrotransposition in hCNS-SCns. (A) Experimental rationale. (B) PCR of genomic DNA; 1,243 bp product contains the intron; 342 bp product indicates intron loss and retrotransposition. Lane 1, weight standards; Lane 2, hCNS-SCns transfected with JM111/L1RP; Lanes 3-5, three hCNS-SCns lines transfected with L1RP, Lane 6-7, primary astrocytes and fibroblasts transfected with L1RP; Lane 8, positive control; Lane 9, water. (C) Southern blot of hCNS-SCns (line FBR-BR3); 2,547 bp band represents plasmid; 1,645 bp band is diagnostic for genomic insertion. (D) Time course of L1 retrotransposition. (E) EGFP-positive cells express Nestin and Sox2. (F) EGFP-positive cells can differentiate to neurons (βIII tubulin and Map2a+2b positive). (G) EGFP-positive cells can differentiate into glia (GFAP positive, βIII tubulin negative). Scale bar=25 μm, arrows indicate co-labeled cell body; arrowheads indicate co-labeled processes.

FIG. 15 is a panel showing L1 retrotransposition in hESC-derived NPCs. (A) Experimental rationale. (B) L1 retrotransposition in H13B (top, LRE3-GFP) and H7 (bottom, LRE3-mneol)-derived NPCs (BF=brightfield). G418-resistant foci can express progenitor (SOX3) and neuronal (βIII tubulin) markers. (C) L1 5′ UTR is induced upon differentiation. (D) H13B-derived NPCs express endogenous ORF1p. RNP=ribonucleoprotein particle samples; WCL=whole cell lysate. (E-G) EGFP-positive, HUES6-derived NPCs express SOX2 and Nestin and can differentiate to be tyrosine hydroxylase (TH) positive. Scale bar=25 μm. LRE-EGFP positive neuron from which (I)-(K) were obtained. Scale bar=10 μm. i, Transient Na+ (asterisk) and sustained K+ currents (arrow) in response to voltage step depolarizations j, Suprathreshold responses to somatic current injections. Spontaneous action potentials (Vm=−50 mV). Arrows indicate cell soma co-localization; arrowheads indicate co-labeled processes.

FIG. 16 is a panel showing methylation analysis and chromatin immunoprecipitation (ChIP) for the endogenous human L1 5′ UTR. (A) Schematic illustrating the L1 CpG island, and SRY/SOX2 binding sites. (B) Cumulative distribution function (CDF) plot, comparing overall methylation and collapsing CpG sites into a single data point, (two-sample Kolmogorov-Smirnov test). (C) Individual methylation of sequences exhibiting highest sequence similarity to consensus RC-L1s. Open circles=unmethylated, closed circles=methylated CpG dinucleotides. (D) ChIP identifying MeCP2 and Sox2 occupying the endogenous human L1 promoter, extracts were analyzed by PCR towards the L1 5′ UTR SRY binding region (Sox2 immunoprecipitation) or CpG island region (MeCP2 immunoprecipitation). CpG dinucleotides exhibited higher methylation at the 5′ end of the CpG island; higher methylation overall was observed in skin samples.

FIG. 17 is a panel showing multiplex quantitative PCR analyses of L1 copy number in human tissues. (A) Experimental schematic. (B-C) Relative quantity of L1, standardized such that the lowest liver value was normalized to 1.0. Hi=hippocampus, C=cerebellum, H=heart, and L=liver. Additional L1 ORF2 assays with other internals controls, FIGS. S9-10. Error bars all s.e.m. *, p<0.05 (repeated measures one-way ANOVA with Bonferroni correction, n=3 individuals, with 3 repeat samples from each tissue). (D) Ten samples from various brain regions (n=3 individuals) compared to somatic liver and heart. One-way t-test, p≦0.0001 with 34 degrees of freedom. Multiplexing of 5S rDNA with α-satellite indicated no significant change, p≦0.5054. Hippocampal tissue compared to liver and heart spiked with estimated plasmid copy numbers of L1 (10, 100, 1,000, and 10,000 copies).

FIG. 18 is a schematic drawing of the rationale for the LINE-1 retrotransposition assay. (A) A cartoon of an RC-L1 is shown at the top of the figure. The dark blue rectangle represents the 5″UTR. The yellow and blue arrows represent ORF1 and ORF2, respectively. The relative positions of the endonuclease (EN), reverse transcriptase (RT) and C-domain (C) in L1 ORF2 are indicated. The 3′ UTR of the L1 was tagged with a retrotransposition indicator cassette, which consists of a reporter gene in the reverse orientation (REP, gray arrow) containing its own promoter and polyadenylation signal. The reporter gene is also interrupted by an intron in the same transcriptional orientation as the RC-L1 (IVS2, black rectangle). This arrangement ensures that the reporter cassette will only be activated and expressed (gray oval) if the spliced RC-L1 mRNA undergoes a successful round of retrotransposition. (B) Several retrotransposition markers (EGFP, top left; NEO, top right; BLAST, below) are useful for studying retrotransposition. Each scheme also indicates the relative position of the primers used to confirm splicing of the intron from the retrotransposition indicator cassette (red arrows). A schematic showing the anticipated results of the PCR-intron removal assay is shown at the right of each figure.

FIG. 19 is a panel showing characterization of L1 retrotransposition events in hCNS-SCns. (A-B) L1-EGFP-positive hCNS-SCns express the neural stem cell markers Musashi1 (nuclear) and SOX1 (nuclear) as well as nestin (cytoplasmic). DAPI, nuclear stain. (C) L1-EGFP-positive hCNS-SCns are still capable of cell division (Ki-67-positive; white, nuclear); Ki-67-positive cells also express the cytoplasmic progenitor marker Nestin. In all images, arrows indicate co-labeled cells. (D) Bright-field images of primary astrocytes and fibroblasts. (E) FACS analyses of hCNS-SCns cells (FBR4, see methods in Examples) transfected with L1_{RP} results in a low, but reproducible rate of L1 retrotransposition. Retrotransposition events were not observed in hCNS-SCns transfected with JM111/L1_{RP} or in primary human astrocytes or fibroblasts transfected with L1_{RP}.

FIG. 20 is a panel showing retrotransposition in hESC-derived NPCs. (A) The schematic at the top of the figures outlines the NPC differentiation protocol used for the H7, H9, H13B and BG01 hESCs. The number of days for each step of differentiation is indicated above the arrows. The medium used in the derivation is indicated below the arrow. Bright-field images of representative cells at each stage of the derivation are shown below the graphic. (B) Dissociated neurospheres express the nuclear neural stem cell markers SOX1 and SOX3. (C) L1-EGFP-positive, HUES6-derived NPCs express SOX1 (nuclear) and Nestin (cytoplasmic). (D) H13B-derived NPCs support L1-EGFP retrotransposition and express SOX3 (nuclear). (E) L1-EGFP-positive, HUES6-derived neurons co-label for the neuronal markers βIII tubulin and Map2a+2b (both cytoplasmic). (F) L1-EGFP-positive, HUES6-derived NPCs can differentiate to a glial lineage that is positive for the cytoplasmic marker GFAP but negative for the neuronal marker βIII tubulin. Arrows indicate co-labeled cells; arrowheads indicate cellular processes that are co-labeled.

FIG. 21 is a panel showing the characterization of L1 retrotransposition in hESC-derived NPCs. (A) FACS analysis of HUES6-derived NPCs transfected with either L1RP or JM111/L1RP. (B) The synapsin promoter is strongly induced upon NPC differentiation. X axis, differentiation time course (days post-differentiation); Y axis, luciferase activity (fold activity). (C-D) Transfection of both HUES6- (C) and H7- (D) derived NPCs with the engineered LRE3-mneoI construct indicates that G418-resistant colonies contain a retrotransposition event, lacking the intron from the indicator cassette. (E) The L1.3 mblastI construct also retrotransposes in HUES6-derived NPCs. (F-G) LRE3 mEGFPI also retrotransposes in both H13B- (F) HUES6-(G) derived NPCs. Molecular size standards are shown at the right of the gel images in panels C-G. Western blot for SOX2 and MeCP2 indicates expression of SOX2 decreases with neural differentiation, whereas MeCP2 expression is upregulated.

FIG. 22 is a panel showing that NPCs exhibit a grossly normal karyotype. (A-C) The three hCNS-SCns cell lines have a normal karyotype. (D) FISH (fluorescence in situ hybridization) using a probe cocktail specifically designed to identify small populations of cells with changes in chromosome 12 and 17 copy number, a common karyotypic abnormality observed in the culturing of hESC29, revealed that HUES-6 cells demonstrated a normal signal pattern for the ETV6 BAP (TEL) gene located on chromosome 12. All cells also demonstrated a normal signal pattern for the chromosome 17 centromere. Two hundred interphase nuclei were examined using this procedure. In sum, we did not detect any evidence of trisomy 12 and/or trisomy 17. (E-F) The HUES6 (E) and H9 ES (F) hESCs exhibit a grossly normal karyotype.

FIG. 23 is a panel showing quantification of L1 RNA transcripts. (A) Quantitative RT-PCR analysis of L1 ORF2 transcripts in in vitro cell types, standardized to actin. (B) RT-PCR analysis of L1 ORF1 transcripts, with GAPDH as a loading control. (C) Quantitative RT-PCR analysis of L1 ORF2 transcripts from fetal brain, skin, and liver, n=3 individuals. (D) RT-PCR analysis of L1 ORF1 transcripts from the same tissues.

FIG. 24 is a panel of analyses of the EGFP-positive and EGFP-negative FACS-sorted NPC populations. (A) PCR on genomic DNA from EGFP-positive and EGFP-negative cell populations revealed L1 retrotransposition events (342 bp product) in the EGFP-positive cells and little of the original LRE3 expression construct (1,243 bp product) in either sample. (B) Characterization of retrotransposition events in hESC-derived NPCs revealed structural hallmarks of LINE-1 retrotransposition. The caricature represents a fully characterized engineered L1 retrotransposition LRE3 event in HUES6-derived NPCs (see Table 3). The schematic shows the sequence of the pre-integration (SEQ ID NO:189) (bottom) and post-integration (SEQ ID NO:190) (top) sites. Also shown are the nucleotide position of the truncation site within L1 (in this example, truncation occurred in the EGFP cassette), the approximate length of the poly (A) tail, target-sited duplications that flank the retrotransposed L1, and the endonuclease recognition site. (C) Both EGFP-positive and EGFP-negative sorted HUES6-derived NPC populations expressed the neural stem cell markers Nestin and SOX2. (D) Both EGFP-positive and EGFP-negative HUES6 derived NPC populations could differentiate to cells of both the neuronal (βIII tubulin) and glial (GFAP) lineages.

FIG. 25 is a panel of methylation analysis of the human L1 5′ UTR. (A-B) The X axis shows the sequence identity (percent) of each L1 5′ UTR analyzed from the brain and skin samples as compared to the database of RC-L1 5′ UTR sequences. The cutoff for analysis was the mean sequence identity to the RC-L1 database minus one standard deviation. The Y-axis shows the percentage of unmethylated CpG dinucleotides in each sample. (C) The conversion of isolated cytosine residues that were not part of a CpG dinucleotide was used to measure the efficiency of the bisulfite conversion reaction. A conversion efficiency of >90% for all analyzed sequences was obtained, with no statistically significant difference between samples (left, D80 female, right, D82 male). (D) The dinucleotide sequences of the L1 5′ UTRs from the brain and skin samples were compared to one another. The only statistically significant difference between brain and skin samples was in the conversion of CpG to TpG dinucleotides, indicating a lesser degree of L1 methylation, in the brain samples when compared to the skin samples. Statistically significant changes were not observed in the first base of any other dinucleotide sequence between the two samples, indicating there was no statistically significant sampling bias of different L1 subtypes between data sets.

FIG. 26 is panel of multiplex qPCR data from human brain areas and somatic tissues. (A) The ratio of ORF2/internal control represents the amount of L1 ORF2 DNA sequence in each sample relative to the amount of L1 5′ UTR, standardized such that the lowest liver value is normalized to 1.0 and all other samples are reported relative to the lowest liver value. Hi=Hippocampus, C=Cerebellum, H=Heart, and L=Liver. Under these conditions, the copy numbers of L1 ORF2 sequences were higher in the hippocampus and, to a lesser extent, the cerebellum when compared to the heart and liver samples. Graphs were obtained by grouping data from different individuals in FIG. 27A. *p<0.05 as a result of a repeated measures one-way ANOVA with a Bonferroni correction (n=3 individuals, with 3 repeat samples from each tissue). (B) Ten additional samples from various brain regions (n=3 individuals) were compared to somatic liver and heart samples (ORF2/5S rDNA). An unpaired t-test comparing grouped brain samples with the somatic tissues, p≦0.002 with degrees of freedom=34. (C) Data from the three individuals were combined for each tissue into a single data point to generate the data in FIG. 4D (Error bars=SEM from 3 different tissue samples).

FIG. 27 is a panel of multiplex qPCR data from hippocampus, cerebellum, liver and heart DNAs isolated from three individuals. (A) Data from the three individuals were combined for each tissue into a single data point to generate the data in FIG. 17B-C and FIG. 26A (n=3 for each tissue in A, n=9 collapsed across the three individuals in FIG. 17). Notably, in two individuals (1079 and 1846), the difference in L1 copy number was evident in multiple multiplex PCR reactions; however, the increase in L1 copy number was more modest in a third individual (4590). (B) Each primer set amplified only a single PCR product, tested on a single hippocampal tissue, 60 cycles of PCR.

FIG. 28 is a representation of a Euclidian distance map based on exon-splicing array data (14). (A) Each cell type was assayed in triplicate and compared to duplicates of human fetal brain standardized RNA (Ambion/Applied Biosystems). In all cases the replicates clustered well together. Fetally derived NPCs clustered closer to HUES6 cells, whereas the HUES6-derived NPCs cluster closer to fetal brain. (B) Promoter analysis of the L1.3 5′ untranslated region indicated two SRY/SOX2 binding sites that were assayed in ChIP experiments. Analysis of a scrambled L1 5′ UTR is included on the right.

FIG. 29 is a flow chart of de novo RT assay procedure. Protocol steps are stated in order following the arrow from top left to bottom right. Tissue is dissected, fresh-frozen on dry ice, and stored at −80 C. Nuclei are isolated from frozen tissue and then sorted via FACS so that one nuclei is in each well of a 96 well microtiter plate. Multiplex quantitative PCR (qPCR) is performed using taqman probes. De novo RT events in brain are quantified relative to heart nuclei from the same individual.

FIG. 30 is a graph showing de novo RT events in three individuals. Cumulative probability plots show the fraction of individual nuclei (y axis) that have an indicated number of de novo RT events (x axis). Each point is the value for an individual cell. The red points are from one individual's hippocampus, blue points are from two individual cortices (one individual in dark blue, the other in light blue). The total number of cells in each sample is indicated by “n=” in the legend. Fold-change in these samples was calculated by rank; in other words the lowest dCt value heart nucleus was paired with the lowest dCt value hippocampal or cortical nucleus, the highest paired with the highest, etc. De novo RT events were calculated by multiplying the fold change by 2382, the number of Orf2 templates identified in the reference genome using BLAT (UCSC).

### DETAILED DESCRIPTION

Various embodiments of methods are provided involving the transposition of retrotransposons, and in particular, non-LTR retrotransposons, in neural cells. As used herein, a “neural cell” is a neuroepithelial cell, a neural stem cell, a neural precursor cell, a neuron, a nerve cell, or a neurocyte. Retrotransposons include both long terminal repeat (“LTR”) retrotransposons and non-LTR retrotransposons. Non-LTR retrotransposons include LINE1 (long interspersed nucleotide elements, or L1) retrotransposons, SINE (short interspersed nucleotide elements) retrotransposons, and SVA (SINE-R, VNTR, Alu) retrotransposons. As is known, L1 retrotransposons are autonomous transposons containing many of the activities necessary for their mobility, while SINE and SVA retrotransposons are non-autonomous elements mobilized by L1 retrotransposons.

In some embodiments, the content of one or more retrotransposons in neural cells or their progeny is compared to the content of one or more retrotransposons in a control neural or non-neural cell. By “content” is meant the amount of DNA encoding a retrotransposon per cell, or the copy number of the retrotransposon per cell.

Some embodiments involve treating non-LTR retrotransposition in a cell. By “treating” is meant to decrease the level of non-LTR retrotransposition occurring in the cell. Although non-LTR retrotransposition may be a normal process of the developing nervous system, in some cases non-LTR retrotransposition can be associated with certain nervous system conditions. In those cases, decreasing non-LTR retrotransposition would be beneficial. In particular cases, the nervous system condition may be affected by, or be the result of, increased non-LTR retrotransposition above that which normally occurs during nervous system development, and in such cases, decreasing non-LTR retrotransposition would also be beneficial. In some embodiments, the treated cell is in a patient, and in such embodiments, “treating” can also mean to lessen the symptoms of a condition, or a total avoidance of a condition, in the patient.

Examples of nervous systems conditions for treatment include, but are not limited to, autism or autism spectrum disorders, schizophrenia, Rett syndrome, Tourette syndrome, ataxia telangiectasia and other ataxias, xeroderma pigmentosum, Cockyne syndrome, fragile x, aspergers syndrome, childhood disintegrative disorder, tuberous sclerosis complex, or neurogiromatosis, Prader-Willi, Angelman, Joubert, Down, Williams and Cowdern syndrome or other psychiatric disorders, or any combination of conditions thereof. A neural cell “identified” with a nervous system condition means the neural cell has a genotype, genetic background, and/or phenotype that causes or predisposes an individual to a nervous system condition.

In embodiments involving treatment, a neural cell is exposed to a retrotransposition inhibitor. The term “expose” means bringing the exterior and/or interior of a cell in contact with the inhibitor. A neural cell in culture can be exposed to a retrotransposition inhibitor by adding the inhibitor to the culture medium. A neural cell in a patient can be exposed to an inhibitor by administering the inhibitor to the patient. The routes of administration will vary, naturally, with the particular patient, condition and retrotransposition inhibitor, and can include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation. For exposure of neural cells in an embryo or fetus in a pregnant patient, routes of administration can include in utero or perinatal administration, injections into the maternal vasculature, or through or into maternal organ including the uterus, cervix and vagina, and into embryo, fetus, neonate and allied tissues and spaces such as the amniotic sac, the umbilical cord, the umbilical artery or veins and the placenta. Both bolus and continuous administration of an inhibitor are contemplated. The dose or quantity to be administered, the particular route and formulation, and the administration regimen are within the skill of those in the clinical arts.

In addition, prior to treatment of a patient, the genotype and/or phenotype of the patient can be determined with respect to the nervous system condition being treated. For example, with respect to Rett syndrome, the MeCP₂ genotype and the Rett syndrome phenotype of the patient can be determined. Similarly, prior to exposure of neural cells in a patient to a retrotransposition inhibitor, the genotype and/or phenotype of the neural cells in the patient can be determined with respect to the nervous system condition being treated. Also, the genotype and/or phenotype of siblings of the patient, or the genotype and/or phenotype of progeny or children of the patient, can be determined with regard to the nervous system condition being treated. Separately or in combination, these determinations can indicate which patient or neural cells in a patient are to be treated or exposed.

Examples of retrotransposition inhibitors include, but are not limited to, an anti-retroviral drug (such as AZT, tenofovir, or nevirapine); an inhibitor of RNA stability; an inhibitor of reverse transcription (such as ddI or ddC); an inhibitor of L1 endonuclease activity; an inhibitor of DNA repair machinery (such as ATM inhibitor CP466722); a zinc-finger that targets the L1 promoter region; an enzyme that inhibits L1 (such as the protein APOBEC3G); and a repressor that inhibit L1 (such as MePC2 and/or Sox2); and any combination thereof.

The retrotransposition inhibitors can be formulated in neutral or salt forms, and with one or more carriers. Pharmaceutically-acceptable salts include the acid addition salts (formed with the free amino groups of the protein) and those which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions can be administered in a manner compatible with the dosage formulation and in such amount as is therapeutically effective to reduce non-LTR retrotransposition. The formulations can be administered in a variety of dosage forms such as injectable solutions, drug release capsules and the like.

The term “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

In embodiments involving the assaying of retrotransposition in neural cells, the content of a retrotransposon in a neural cell is compared to the content of the retrotransposon in a control cell. The nature of the control cell depends on the type of comparison. For example, the assayed neural cell can be a mutant neural cell, such as an MeCP₂ knockout (“KO”) cell, while the control cell is an MeCP₂ wild type neural cell. Alternatively, the control cell can be a non-neural cell, such as a fibroblast, a heart cell, a hepatocyte, a muscle cell, or another non-neural cell.

Embodiments that involve identifying an inhibitor of retrotransposition also compare the content of a retrotransposon in a neural cell to a control cell. In these embodiments, the control cell is typically a similar type of neural cell that has not been exposed to the inhibitor, and can be of the same or comparable genetic background as the neural cell.

In embodiments involving the identification of a neural condition associated with non-LTR retrotransposition by comparison to control cells, the nature of the control cell depends on the type of comparison. For example, the assayed neural cell can be a mutant neural cell, such as an MeCP₂ knockout (“KO”) cell, while the control cell is an MeCP₂ wild type neural cell. Alternatively, the control cell can be a non-neural cell, such as a fibroblast, a heart cell, a hepatocyte, a muscle cell, or another non-neural cell. In addition, the content of the neural cell can be determined by specific methods such as copy number determination by polymerase chain reaction, or by measuring the hybridization signal of a probe. The identification of a neural condition associated with non-LTR retrotransposition can be part of a subject's diagnostic or treatment regimen, where the diagnosed neural condition is then treated by exposing the subject's neural cells to a transposition inhibitor in an amount sufficient to decrease non-LTR retrotransposition in the neural cells.

The neural cells in various embodiments can be in culture, in an organ, or in an individual.

It is contemplated that any method or composition described herein can be implemented with respect to any other method or composition described herein. Also, the use of the word “a” or “an” when used in conjunction with the term “comprising” in the claims and/or the specification may mean “one,” but is also consistent with the meaning of “one or more,” “at least one,” and “one or more than one.”

### Example 1

### Methods

### Cell Culture and Transfection

Rat NSCs were isolated, characterized and cultured as described (31,32). For neuronal differentiation, cells were cultured in N2 medium (Invitrogen) containing retinoic acid (RA, 1 μM, Sigma) and forskolin (5 μM, Sigma) for 4 days (11). Freshly isolated neuroepithelial cells from time-pregnant midgestation (E11.5) telencephalons from WT and MeCP2 KO sibling mouse embryos from the same background (C57BL/6J) were briefly cultured for 2-3 passages in FGF-2 as described elsewhere (19). Primary skin fibroblasts were isolated from tail biopsy and cultured in DMEM (Invitrogen) with 10% FBS (Invitrogen). Samples of all isolated cells were used for genotyping using previously described primers (33). Plasmid transfections were done by electroporation following the manufacturer's instructions (Amaxa Biosystem).

In Vitro Methylation, Luciferase Assay and siRNA Sequences

The L1 5′UTR-Luc plasmid was methylated by Hpa II (NEB) according to the manufacturer's protocol. Complete methylation was checked by digestion with Hpa II restriction enzyme. Luciferase activity was measured with the Dual-Luciferase reporter assay system (Promega) according to the manufacturer's protocol. Luciferase activity was usually measured 48 h after transfection. A plasmid containing the Renilla luciferase gene was used as an internal control. All the experiments were done at least 3 times independently and transfection efficiency was about 30% for all samples. The siRNAs used in this study were purchased from Dharmacon and used according to the manufacturer's protocol.

### Chromatin Immunoprecipitation (ChIP)

ChIP assay was done essentially following the manufacturer's protocol using a ChIP assay kit (Upstate). Antibodies used were anti-MeCP2 (Upstate), Sox2 (Chemicon), and IgG. Purified DNA was amplified by PCR using primers for the rat L1 5′UTR promoter region (L1.3, accession # X03095).

### Bisulfite Analysis

Genomic DNA from NSCs was isolated using standard phenol-chloroform extraction techniques. Subsequently, DNA was digested with the restriction enzyme EcoRI and the bisulfite conversion reaction was performed using the Epitect kit (Quiagen), following manufacturer's instructions. Primers were designed based on the rat L1 sequence Mlvi2, using Methyl Primer Express; primers for L1 converted 5′UTR region: forward 5-AACAAAGTAACACTAGAGATAA-3′ (SEQ ID NO:1) and reverse 5′-TTTGGTGGGAGAATTGGGCT-3′ (SEQ ID NO:2). PCR products were cloned into TOPO TA 2.1 plasmids (Invitrogen) and 40 bacterial colonies were analyzed by sequencing.

### Immunofluorescence, Immunoblotting and Co-Immunoprecipitation

Immunofluorescence for EGFP was performed as previously described (11). A non-transgenic animal was used to measure the background fluorescence in the brain and to establish a threshold for detection. Western blotting was carried out using standard protocols with the following antibodies: mouse anti-Actin (1/500, Ambion), rabbit anti-MeCP2 (1/1000, Imgenex or Upstate), and rabbit anti-Sox2 (1/100, Chemicon). All secondary antibodies were purchased from Jackson ImmunoResearch. For co-immunoprecipitation, the Nuclear Complex Co-IP kit (Active Motif) was used, following the manufacturer's protocol with the highest stringency buffers.

Single-Cell Genomic Quantitative PCR (qPCR)

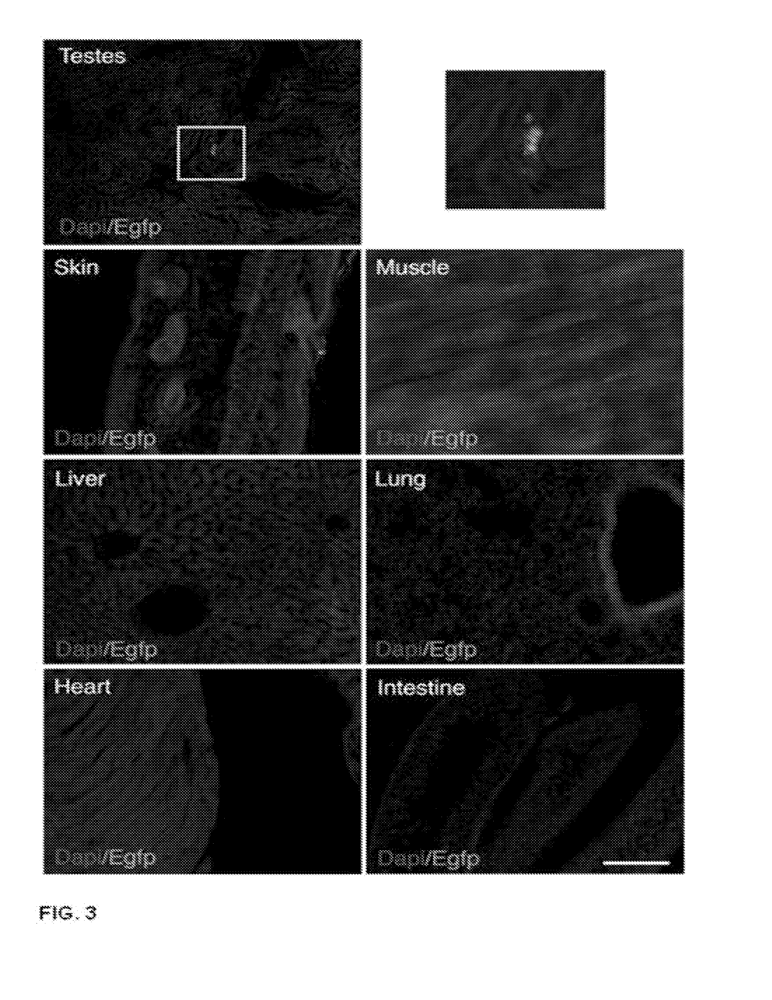

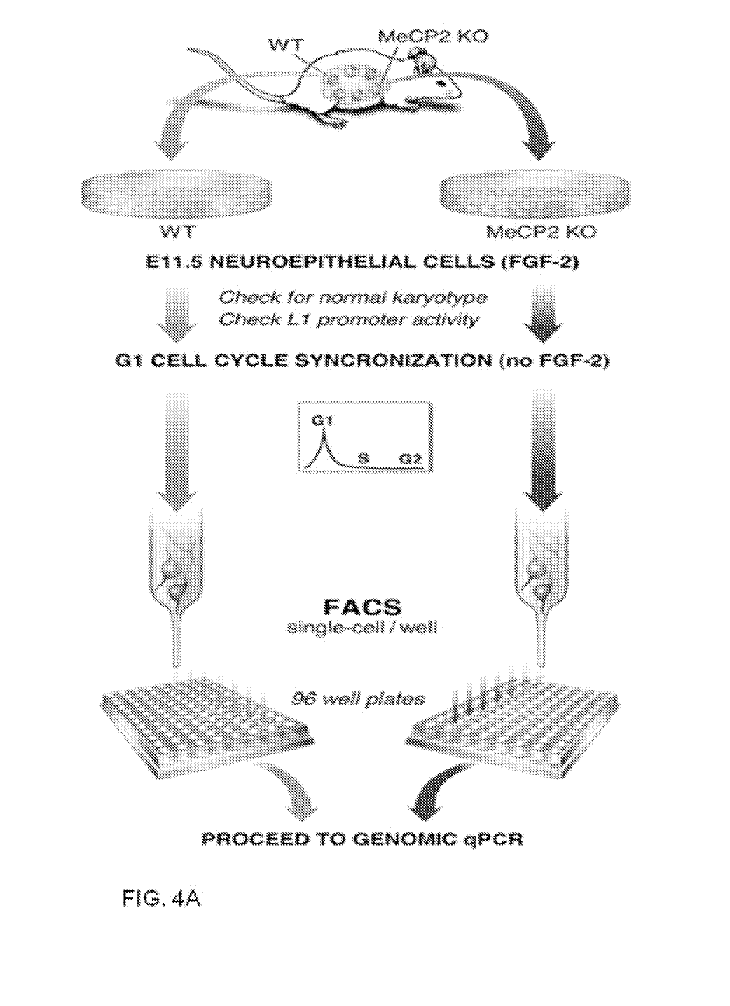

Cell cycle-arrested cells were subjected to a Fluorescent Activated Cell (FACs) sorting in which matched passage number single cells were sorted into an Optical 96-well reaction plate (MicroAmp™-Applied Biosystems, CA) suitable for use in Real Time PCR. The plates containing 1 cell/well were then snap frozen at −70° C. until the day of the qPCR. The qPCR was performed using the protocol available on the manufacturer's website. Briefly, a solution containing forward/reverse primers and SYBR® Green PCR Master Mix was added to the previously sorted cells and the detection of DNA products was carried out in a ABI PRISM® 7700 Sequence Detection System. The SYBR green dye fluoresces only upon binding to the minor groove of double stranded DNA. Thus, the mass of DNA generated by PCR could be quantified and verified by a dissociation (melting) curve analysis that was performed using the Dissociation Curves application software. The primers used for qPCR were designed using specifications on the Primer Express software. Computational estimates using BLAT (May 2006 mouse genome assembly, see Methods) indicated that at least 1,290 endogenous active L1 elements could be detected using ORF2 primers (ORF2-F: 5′-ctggcgaggatgtggagaa-3′ (SEQ ID NO:3), ORF2-R: 5′-cctgcaatcccaccaacaat-3′ (SEQ ID NO:4)). Primers were designed to amplify a product of 52-57 bp. Amplicons of the predicted size were detected in most single cells analyzed (FIG. 3E). Sequence analysis from the cloned PCR product revealed several L1 elements, mostly from full length, retrotransposition competent, mouse T_{F} and G_{F} families (17). Several control primers for the mouse L1 5′UTR were used, giving similar results (primer set A: 5′UTR-F: 5′-taagagagcttgccagcagaga-3′ (SEQ ID NO:5), 5′UTR-R: 5′-gcagacctgggagacagattct-3′ (SEQ ID NO:6); primer set B: 5′UTR-F: 5′-agagagcttgccagcagagagt-3′ (SEQ ID NO:7), 5′UTR-R: 5′-gcagacctgggagacagattct-3′ (SEQ ID NO:8), primer set C: 5′UTR-F: 5′-tgtctcccaggtctgctgataga-3′ (SEQ ID NO:9), 5′UTR-R: 5′-gattgttcttctggtgattctgttacc-3′ (SEQ ID NO:10)). These experiments were reproduced several times, with isolated cells from two different mice for both MeCP2 KO and WT backgrounds.

Multiplex qPCR in Human Tissues

Oligonucleotide PCR primers and TaqMan-MGB probes were designed using Primer Express software (Applied Biosystems). Primers were purchased from Allele Biotech, and probes were purchased from Applied Biosystems. L1 primers were verified using the L1 database (http://llbase.molgen.mpg.de/) and matched at least 140 of 145 identified full length retrotransposition competent L1s. Human tissues were obtained from the NICDH Brain and Tissue Bank for Developmental Disorders at the University of Maryland, Baltimore, Md. Patients were between 17 and 22 years of age. Human genomic DNA was extracted and purified from human tissues using a Blood & Tissue kit (Quiagen), according to the manufacturer's instructions. PCR reactions were carried out using 80 pg of DNA and were verified empirically as amplifying with a CT between 20 and 25 (n=16). Quantitative PCR experiments were performed using an ABI Prism 7000 sequence detection system and Taqman Gene Expression Mastermix from Applied Biosystems. Data analysis was performed using the SDS 2.3 software (Applied Biosystems). The multiplexing reaction was optimized by limiting reaction components until both reactions amplified as completely as each individual reaction. Primer efficiency was verified using a PCR standard curve of plasmid DNA to have a slope of near −3.32. Standard curves of genomic DNA ranging from 2 ηg to 3.2 pg were performed to verify that the 80 pg dilution used was within the linear range.

### Primers and Probes

### L1 Primers

ORF2-1 primers match 5,543 L1's in the genome, and align with 4,560 L1 sequences in the genome. These primers match 144 of the full length L1's in an L1 database (on the Internet at llbase.molgen.mpg.de/). ORF2-1 probe: ctgtaaactagttcaaccatt (SEQ ID NO:11), ORF2-1F: 5′-tgcggagaaataggaacactttt-3′ (SEQ ID NO:12), ORF2-1R: 5′-tgaggaatcgccacactgact-3′ (SEQ ID NO:13). ORF2-2 primers match 3,447 L1's in the genome and align with 2,918 L1 sequences in the genome. ORF2-2 probe: 5′-aggtgggaattgaac-3′ (SEQ ID NO:14), ORF2-2F: 5′-caaacaccgcatattctcactca-3′ (SEQ ID NO:15), ORF2-2R: 5′-cttcctgtgtccatgtgatctca-3′ (SEQ ID NO:16). L15′UTR probes: L15′UTR-1 primers match 1,299 L1's in the genome, and align with 965 μl sequences in the genome. L15′UTR-1 probe: 5′-aaggcttcagacgatc-3′ (SEQ ID NO:17), L15′UTR-1F: 5′-gaatgattttgacgagctgagagaa-3′ (SEQ ID NO:18), L1 5′UTR-1R: 5′-gtcctcccgtagctcagagtaatt-3′ (SEQ ID NO:19). L15′UTR-2 primers match 1,442 L1's in the genome, and align with 876 μl sequences therein. L15′UTR-2 probe: 5′-tcccagcacgcagc-3′ (SEQ ID NO:20), L15′UTR-2F: 5′-acagctttgaagagagcagtggtt-3′ (SEQ ID NO:21), L15′UTR-2R: agtctgcccgttctcagatct-3′ (SEQ ID NO:22).

### Primers Toward Genomic Controls

Satellite alpha (SATA) primers match millions of tandem copies in the genome, with little sequence variability. SATA-probe: 5′-tcttcgtttcaaaactag-3′ (SEQ ID NO:23), SATA-F: 5′-ggtcaatggcagaaaaggaaat-3′ (SEQ ID NO:24), SATA-R: 5′-cgcagtttgtgggaatgattc-3′ (SEQ ID NO:25). There are 47 copies of the 5S ribosomal RNA gene found in the genome, with 35 probe matches. 5S RNA-probe 5′-agggtcgggcctgg-3′(SEQ ID NO:26), 5S-F: 5′-ctcgtctgatctcggaagctaag-3′ (SEQ ID NO:27), 5S-R: 5′-gcggtctcccatccaagtac-3′ (SEQ ID NO:28). There are 316 primer matches to human endogenous retrovirus H (HERVH), a repetitive non-mobile element in the genome, with 99 probe matches. HERVH-probe: 5′-cccttcgctgactctc-3′ (SEQ ID NO:29), HERVH-F: 5′-aatggccccacccctatct-3′ (SEQ ID NO:30), HERH-R: 5′-gcgggctgagtccgaaa-3′ (SEQ ID NO:31).

### Animals and Tissue Preparation

MeCP2 KO mice (33) were obtained. The generation of the L1-EGFP animals has been previously described (11). The L1-EGFP transgene was incorporated in the MeCP2 KO background by crossing L1-EGFP males to MeCP2+/− females. Six gender-matched mice, from the same C57BL/6J background, were used per group. Tissues were prepared from adult animals (8 weeks old) as previously described (11). Quantification of EGFP-positive cells in whole brain slices was done by individuals blinded to mice genotypes. EGFP-positive cells were counted in a one-in-six series of sections (approximately 240 μm apart). Images were taken by a z-step of 1 μm using a Biorad radiance 2100 confocal microscope. All experimental procedures and protocols were approved by the Animal Care and Use Committees of The Salk Institute, La Jolla, Calif.

### 3D Brain Reconstruction

Whole slides containing multiple 40-μm sections of brain from a representative individual from each genetic background were scanned on an iCyte™ fluoro-chromatic imaging cytometer (CompuCyte Corperation, Cambridge, Mass.). The iCyte is a research imaging cytometer based on an inverted platform utilizing lasers, photomultiplier tubes and a scatter detector. The stained slides were scanned at 20× using an argon laser. Sequential images were captured across the entire slide in the X, Y and Z planes. Image processing was completed using iBrowser™ (CompuCyte Corperation) software to stitch the images together into a high-resolution single image of the entire slide. Image Pro Plus™ software (MediaCybernetics, Silver Springs, Md.) was used to extract the images of individual serial sections of brain. The stained cells were manually tagged using Image Pro to identify the X and Y coordinates prior to performing three-dimensional reconstruction. Three-dimensional brain reconstruction was possible using MATLAB software expanded with the MATLAB Image Processing and Virtual Reality Toolboxes. A combination of small, custom programs designed to automate the process of reconstruction from two-dimensional slice images first performed an image contrast calibration on the image stack to increase the illumination of each slice with respect to the slide background. Then, imaged slices were ordered to correspond to a two-dimensional coronal representation of the Allen Reference Atlas. A MATLAB algorithm performed an approximate overlay of the positive cells onto the reference atlas slices, and then the complete data set was converted into Virtual Reality Modeling Language (VRML) format for three-dimensional display. This method was favored over a three-dimensional reconstruction of the actual imaged slices because tears and imperfect alignment of the original slices produced a reconstruction that was more difficult to interpret.

### Results

### MeCP2 is Part of a L1 Promoter Repressor Complex in NSCs

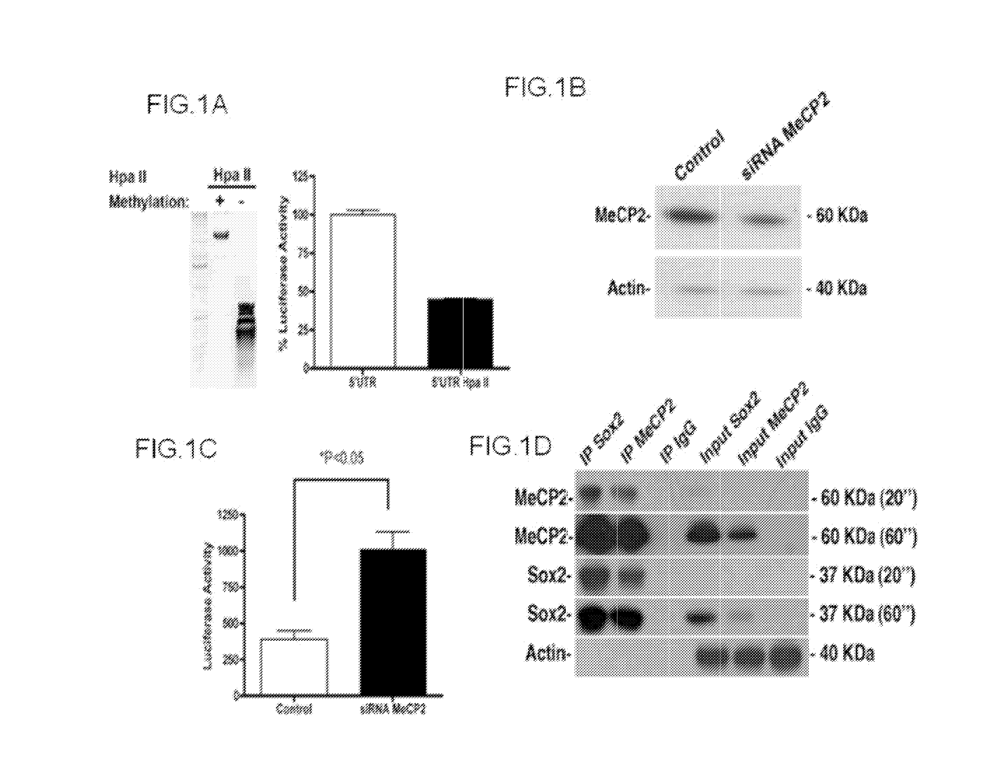

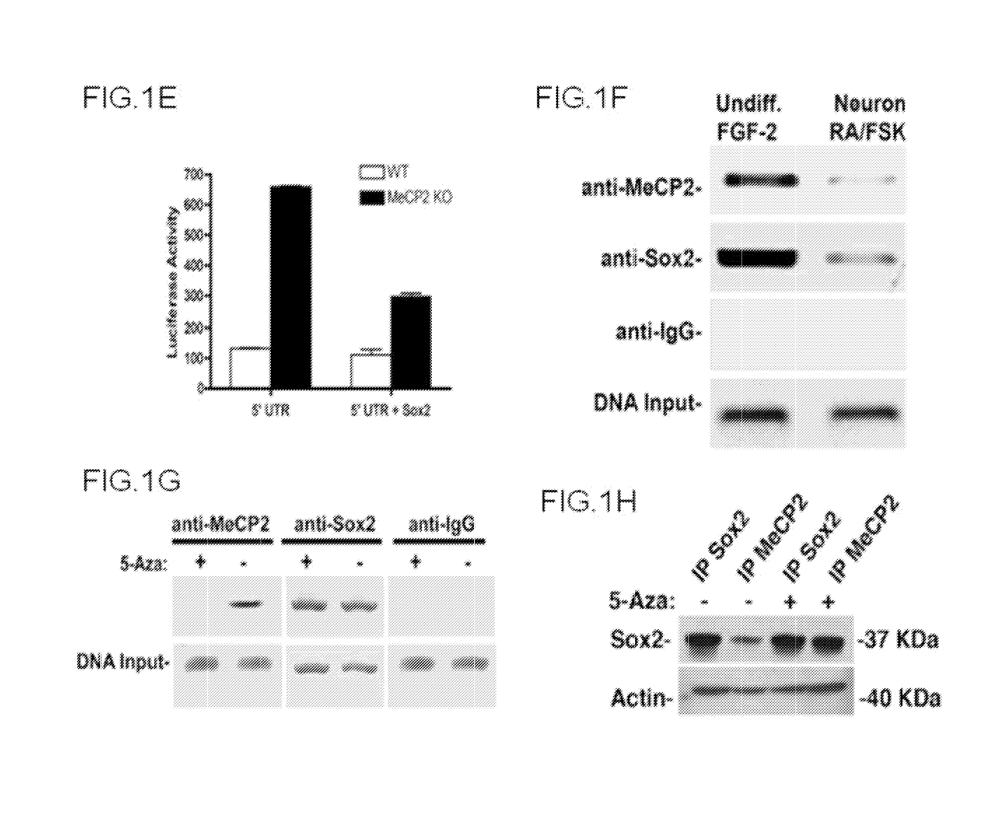

In rat undifferentiated NSCs, the repressor complex in the L1 5′UTR includes the transcriptional factor Sox2 and the histone deacetylase 1 (HDAC1) protein (11), a well-characterized partner of MeCP2 (14,15). MeCP2 was shown to bind to methylated CpG islands in the L1 promoter and reduce retrotransposition in an artificial, non-neural in vitro system (16). Therefore, the role of MeCP2 in the promoter activity of L1 elements in rat NSCs cultured in the presence of FGF-2 was investigated. For that purpose, the human L1 5′UTR promoter region was cloned upstream to the luciferase gene, generating the L1 5′UTR-Luc plasmid (11). Reduction of MeCP2 protein levels by approximately 65%, using specific siRNA against MeCP2 transcripts, led to a 3-fold increase in luciferase activity from the in vitro methylated L1 5′UTR-Luc plasmid (FIG. 1A-C), a finding that is consistent with the idea that endogenous MeCP2 represses L1 expression in NSCs.

Immunoprecipitation of MeCP2 in protein extracts isolated from rat NSCs revealed Sox2 enrichment in Western blot analysis. The reverse was also true: immunoprecipitating Sox2 enriched the detection of MeCP2 in NSCs (FIG. 1D). This result suggests that MeCP2 and Sox2 can be part of the same L1 repressor complex in undifferentiated cells. Additionally, transfection of the L1 5′UTR-Luc methylated plasmid in freshly isolated neuroepithelial cells from C57BL/6J mouse embryonic (E11.5) brain revealed that the L1 promoter activity was approximately 6 times more active in the MeCP2 KO cells when compared to wild-type (WT), confirming that MeCP2 is able to repress the L1 5′UTR promoter activity (FIG. 1E). Overexpression of Sox2 reduced the luciferase activity in MeCP2 KO cells by half, indicating that both Sox2 and MeCP2 are important for silencing L1 expression in NSCs and reflects a MeCP2-independent role for Sox2 (FIG. 1E).

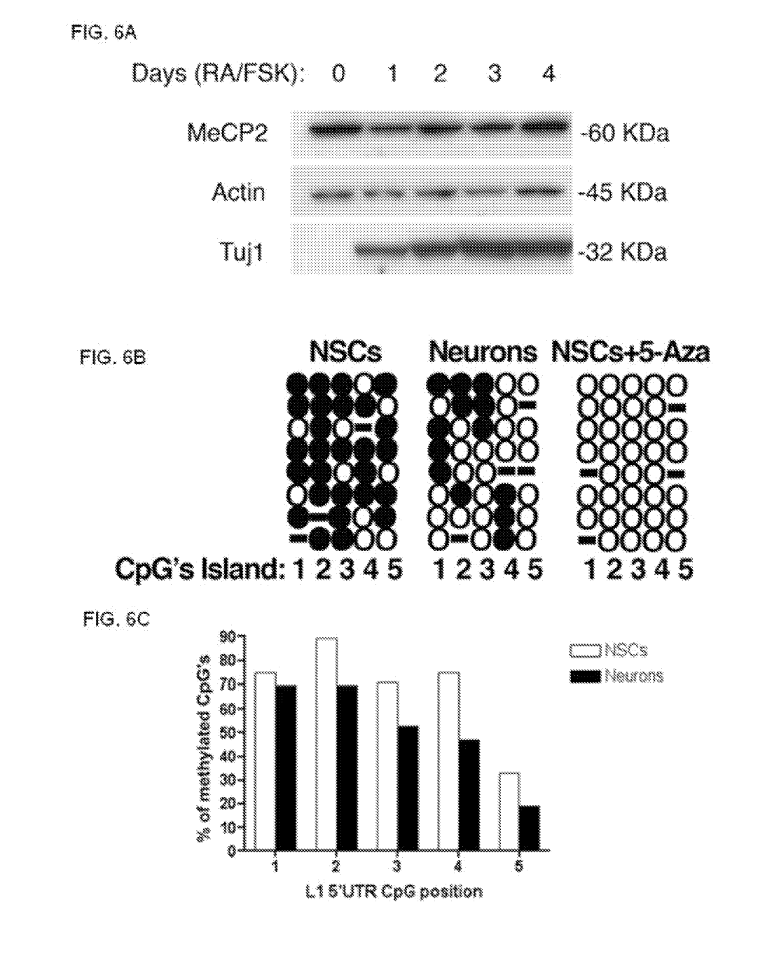

Using chromatin immunoprecipitation (ChIP), high levels of MeCP2 were detected in association with the L1 promoter region (i.e., 5′UTR) in rat NSCs compared to differentiated neurons (FIG. 1F). To rule out differences in protein abundance that may differently influence ChIP amplification, western blot analysis was performed using lysates from cells that had undergone neuronal differentiation. In our four-day system, MeCP2 protein levels did not dramatically change during neuronal differentiation, suggesting that differential amplification was not accounted for by differences in protein recruitment to the L1 promoter (FIG. 6). Interestingly, it was observed that the L1 promoter has a tendency to de-methylate CpG islands close to the transcription starting point during neuronal differentiation, indicating that DNA methylation may silence L1 expression by attracting MeCP2 (FIG. 6). After NSCs treatment with 500 ng/ml of 5-Azacytidine (5-Aza, a global demethylating agent) for 4 days, the MeCP2 ChIP signal was lost, demonstrating that binding was dependent on DNA methylation. In contrast, treatment with 5-Aza did not prevent Sox2 binding to the L1 promoter, suggesting that the association of Sox2 to the DNA does not require the presence of MeCP2 in the methylated site (FIG. 1G). Next, it was asked if releasing of MeCP2 from the methylated DNA would increase its association with Sox2 in solution. The Co-IP experiment in the presence of the 5-Aza was then repeated. Indeed, the 5-Aza treatment increased the amount of MeCP2 in association with Sox2 in solution (FIG. 1H). Taken together, these data suggest that MeCP2 may be associated with the L1 promoter either via DNA methylation and/or by interaction with the Sox2 repressor complex. It may be that both configurations can occur on the same L1 promoter region or each possibility is a L1 element sequence-dependent event.

### MeCP2 Regulates L1 Retrotransposition In Vivo

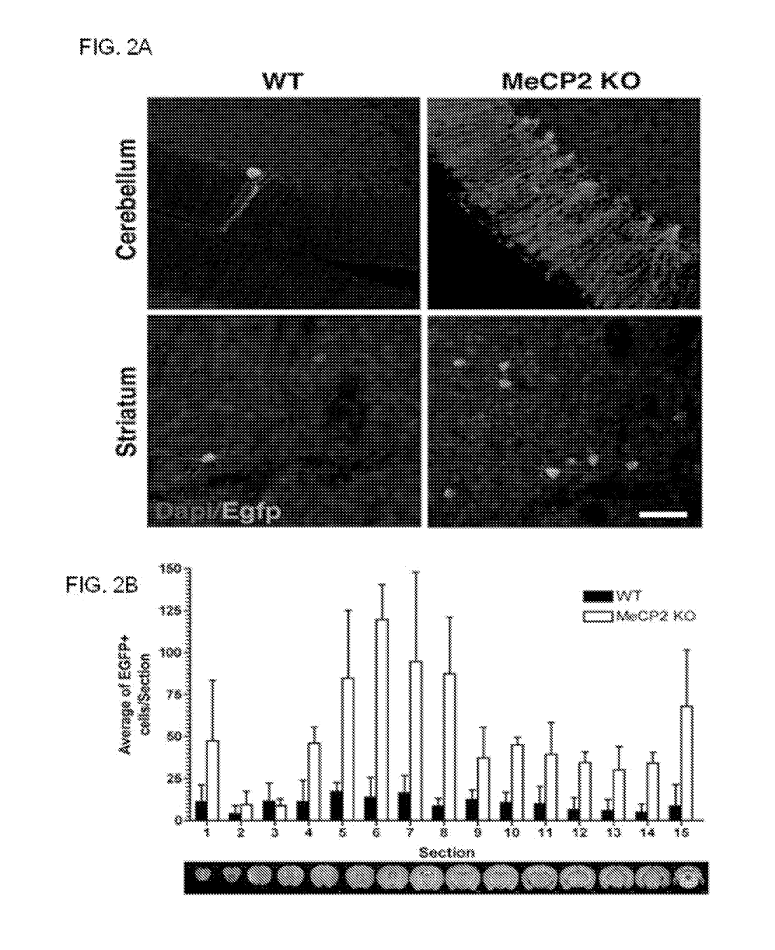

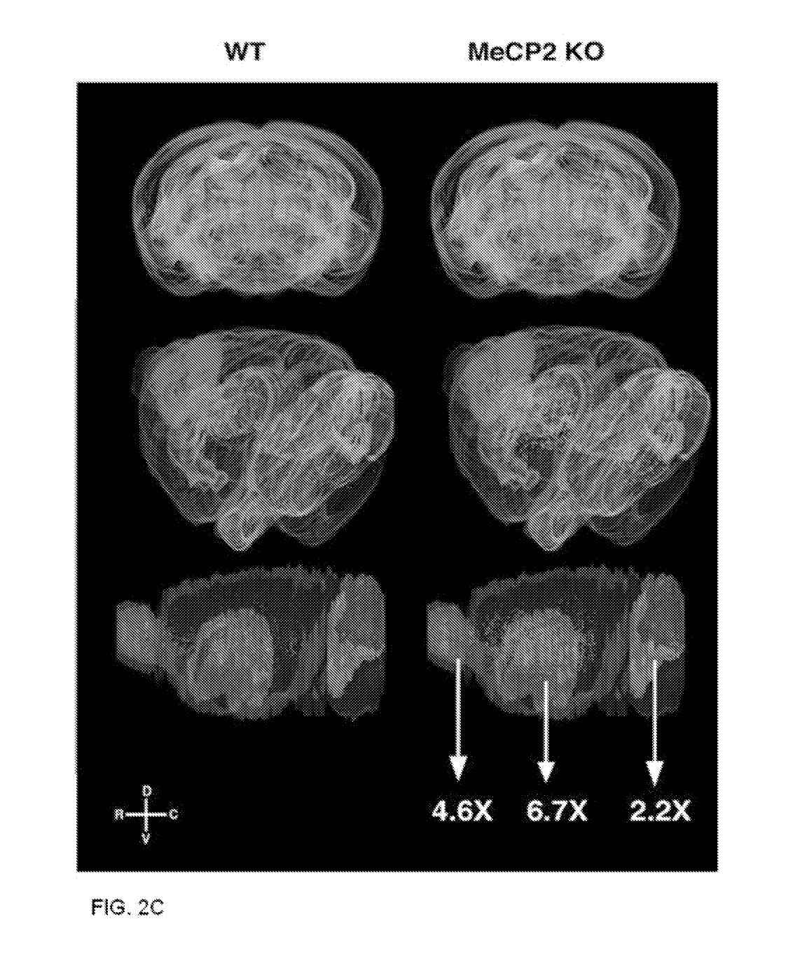

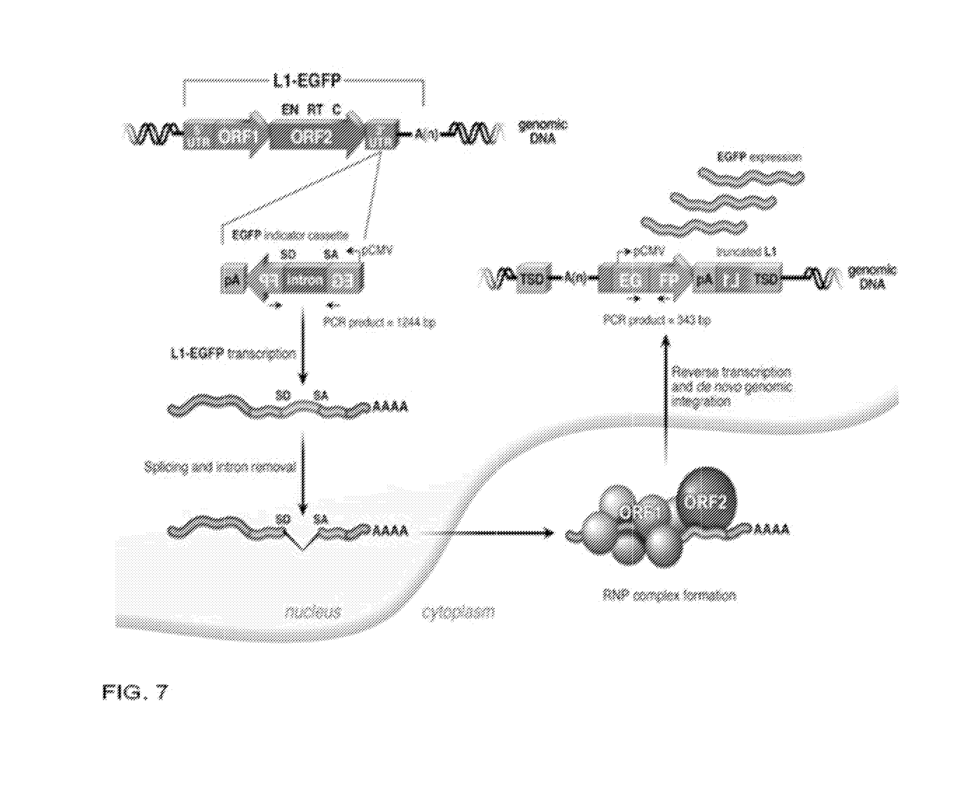

To study L1 regulation by MeCP2 in vivo, the MeCP2 KO mouse model was used to compare the brains of the L1-EGFP transgenic mice in WT and MeCP2 KO genetic backgrounds. The L1-EGFP transgenic mice have an L1 indicator cassette that will only activate the expression of the EGFP reporter after retrotransposition (11) (FIG. 7). In both genetic backgrounds, EGFP-positive cells in the brain co-localized with the mature neuronal marker NeuN and were detected in several regions, for example, in different cortical layers, indicating that L1 retrotransposition probably occurred in NPCs at different times during brain development (data not shown). However, the numbers of EGFP-positive cells in the brains of MeCP2 KO mice were significantly higher compared to WT (FIG. 2). Moreover, EGFP-positive cells were also observed in the germ line of MeCP2 KO at similar frequency to WT animals, but not in other somatic tissues, indicating that the repressor effect of MeCP2 in L1 retrotransposition was restricted to the neural system (FIG. 3). This result confirms that the frequency of L1 retrotransposition is probably higher in the neural system than in other somatic tissues (11).

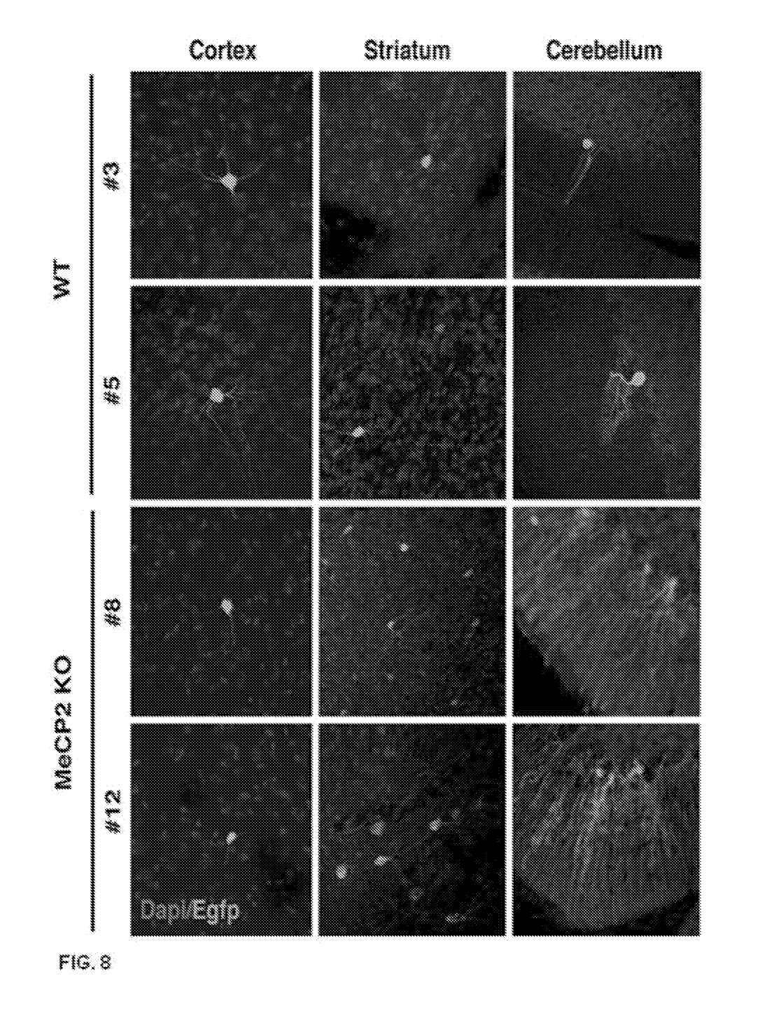

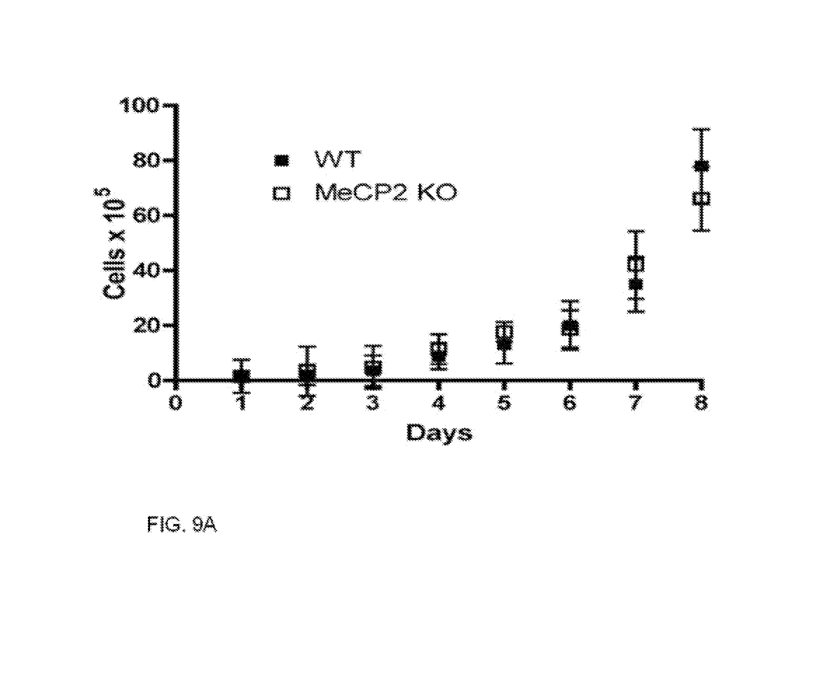

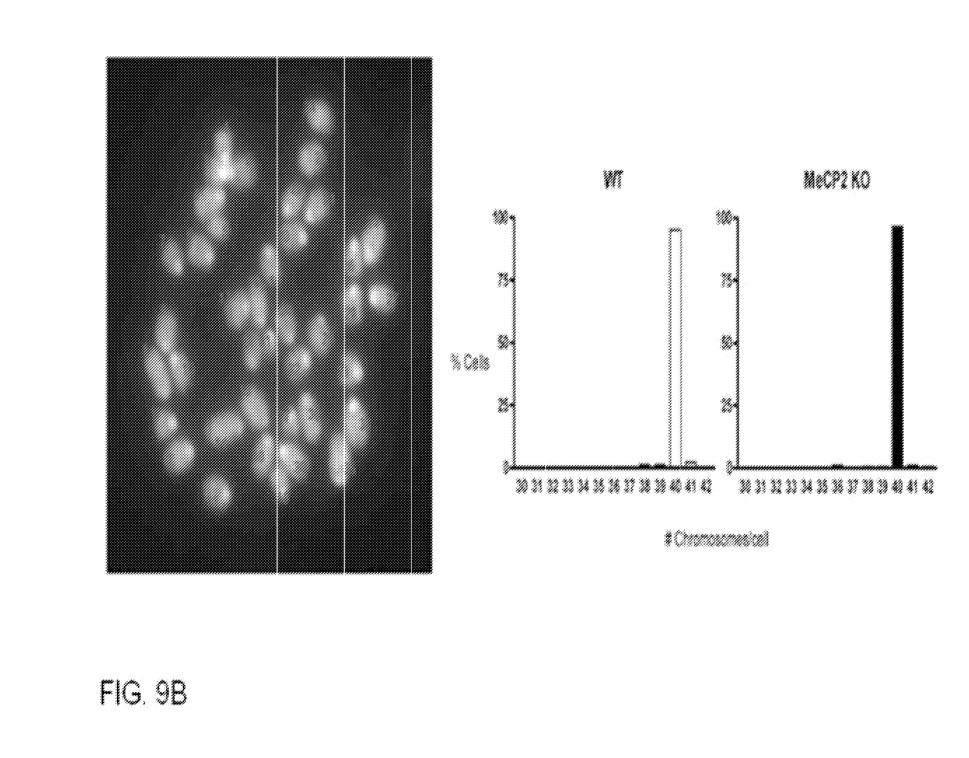

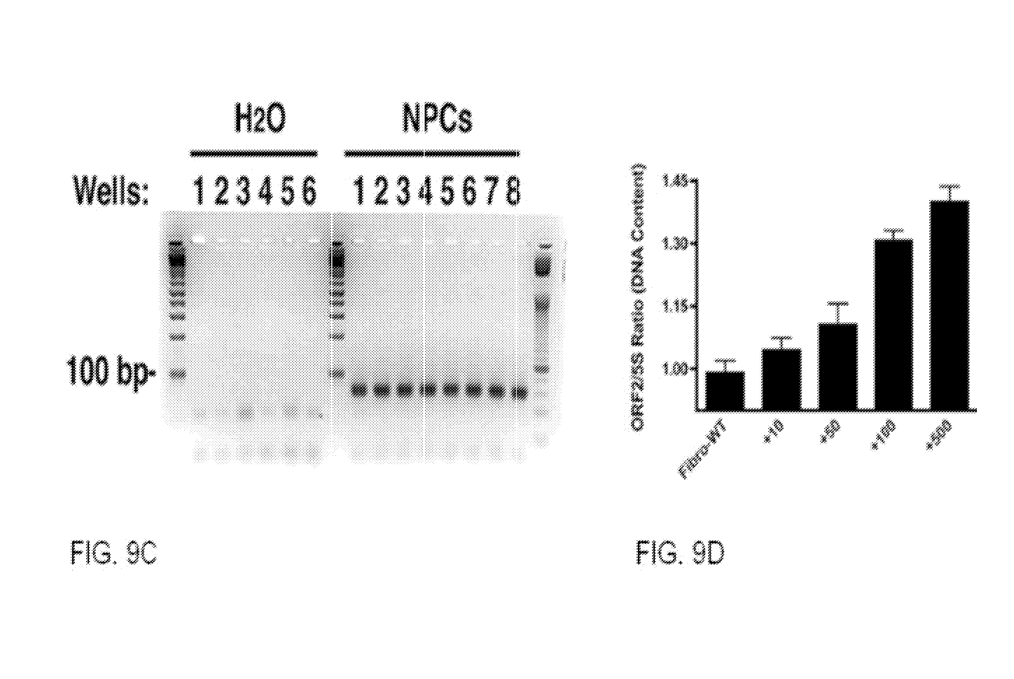

To better visualize the distribution of EGFP-positive cells in the brain, high-resolution, three-dimensional maps of both MeCP2 KO and WT brains were generated. Analysis of six brains and a representative three-dimensional brain reconstruction indicated that, despite MeCP2 KO brain sections had an average of 3.5-fold more EGFP-positive cells than WT, certain brain structures are more prone to L1 retrotransposition. Specifically, the cerebellum, striatum and olfactory bulb contained 2.2±0.5, 6.7±1.2 and 4.6±1.7-fold more EGFP-positive neurons, respectively, in the MeCP2 KO genetic background compared to WT (FIG. 2C). Moreover, EGFP-positive neurons were often observed in clusters of specific types of neurons, such as Purkinje cells in the cerebellum or interneurons in the striatum (FIG. 2A and FIG. 8). Additionally, analyses of striatum and neocortical EGFP-positive cells indicated that most L1-mediated retrotransposition events occurred from E12 to E16, whereas the presence of EGFP-positive cells in the cerebellum indicated that retrotransposition continued postnatally. Finally, the distribution of EGFP-positive cells in the brains of different genetic backgrounds was distinct. Several clusters of EGFP-positive cells could be found in the MeCP2 KO compared to a more homogeneous distribution in the WT brains. More EGFP-positive cells in the MeCP2 KO background may suggest an increased rate of L1 retrotransposition. It could also indicate an earlier retrotransposition event that marked multiple daughter cells and/or that MeCP2 is involved in EGFP silencing after L1 retrotransposition. In culture, no evidence was found that neuroepithelial cells from the MeCP2 KO genetic background have a higher rate of division or expansion when compared to WT cells, suggesting that the increase number of EGFP-positive cells may not be related to an increase in cell proliferation (FIG. 9).

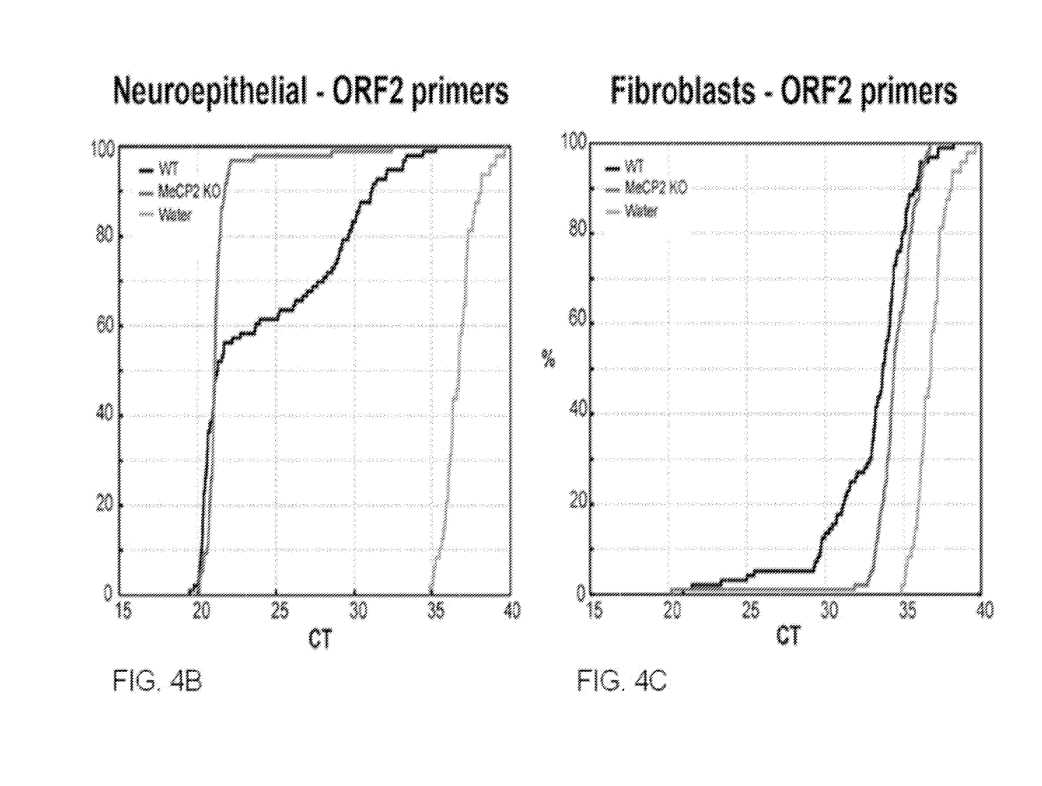

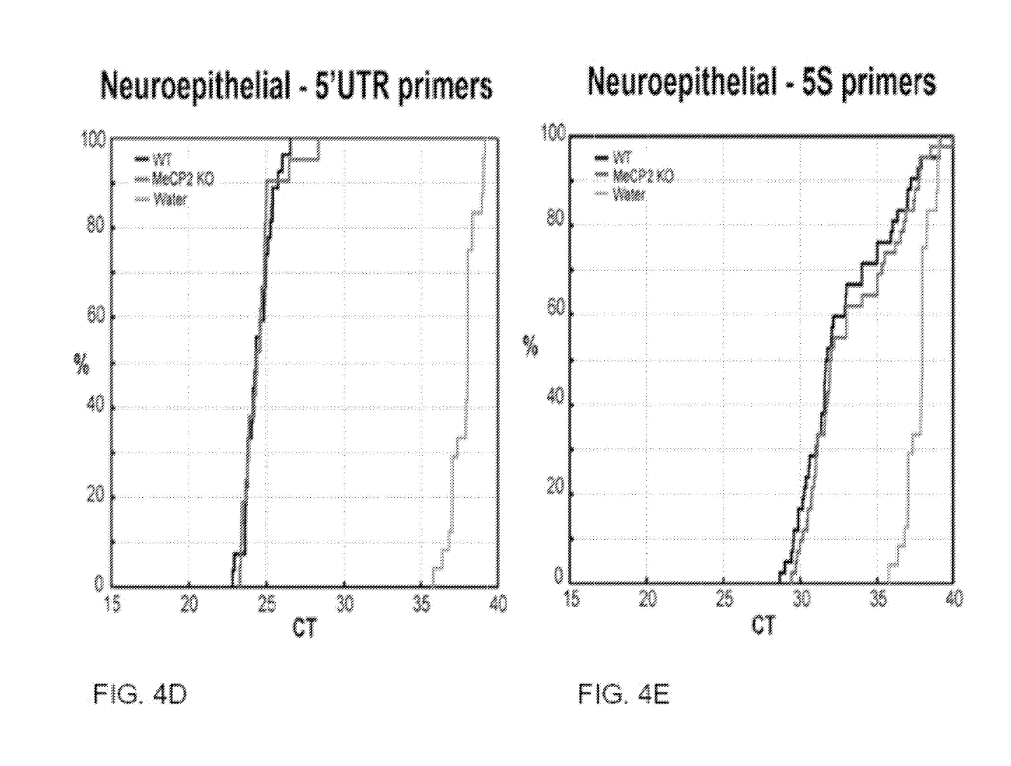

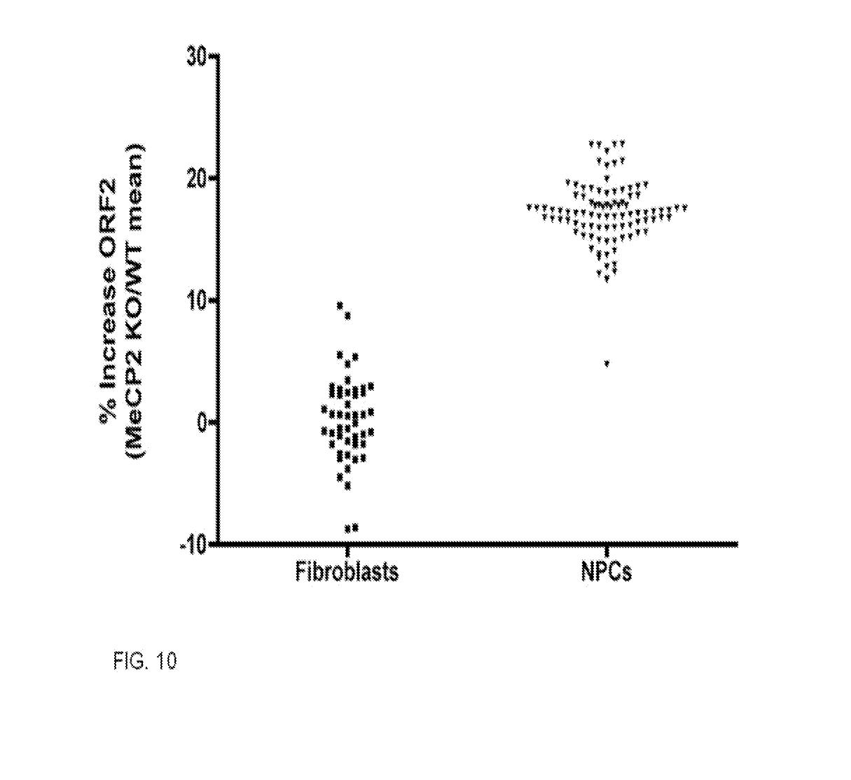

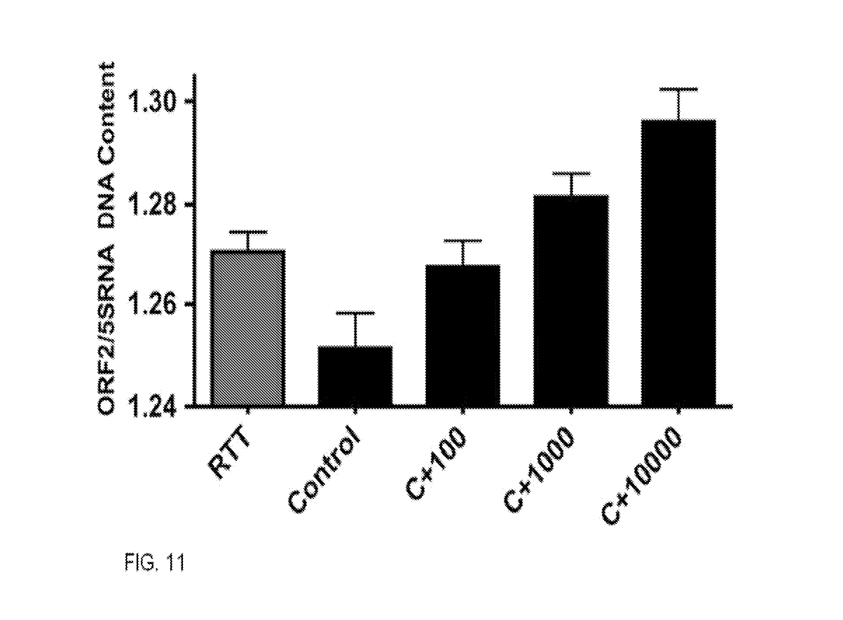

Next asked was whether endogenous L1 retrotransposition is indeed increased in the MeCP2 KO brain. Although the L1-EGFP transgenic animals provide an accessible way to visualize L1 retrotransposition in vivo, they only represent the activity of a single L1 element. In contrast, the mouse genome is estimated to contain at least 3,000 active L1s located on different chromosomes and subject to distinct chromatin context regulation (17,18). To confirm that the L1-EGFP behaves similar to the endogenous L1 elements, a technique was developed based on single-cell genomic quantitative PCR (qPCR) that measures the frequency of mouse L1 sequences (FIG. 4A). It was hypothesized that MeCP2 KO-derived neuroepithelial cells have increased genomic content of L1 sequences compared to WT cells, due to new insertional events. Because of the neuronal specificity of MeCP2, the increased L1 DNA content should be mostly detected in NPC but not in other somatic cell types, such as fibroblasts (FIG. 3). Freshly isolated neuroepithelial cells from time-pregnant midgestation (E11.5) telencephalons with the same background strain (C57BL/6J) were briefly cultured in FGF-2 as described elsewhere (19). At this stage, most cells are committed to the neuronal lineage upon FGF-2 withdrawal (19,20) and are likely to support L1 retrotransposition (11). Cells from WT and MeCP2 KO sibling mouse embryos were synchronized in G1 phase by removal of FGF-2 before sorting. Cells were also karyotyped to ensure chromosomal genetic stability, since major chromosome aberrations, such as aneuploidy, would interfere with genomic L1 detection (FIG. 7). Finally, single-cell amplification using primers designed for ORF2 region from modern active L1 families was confirmed by the presence of the expected amplicons. The qPCR was sensitive to measure variations of up to 10 or more copies/cell (FIG. 9). The use of C57BL/6J sibling animals and synchronized single cell comparison reduced potential artifacts generated by differential genomic backgrounds and DNA replication. Since the cycle threshold (CT) values obtained by qPCR for L1 content were not normally distributed, as can be observed from the cumulative distribution plots in FIG. 4B-D, the non-parametric Kolgomorov-Smirnov (two-tailed) test was applied with the null hypothesis that the CT values for L1 content in different cell types were drawn from a similar distribution. As a result, it was found that the CT values were likely drawn from distinct distributions. Under these conditions, MeCP2 KO-derived neuroepithelial cells displayed significantly (P<0.001) more ORF2 genomic copies when compared to WT cells, suggesting more L1 insertions per cell. In fact, the difference in genomic L1 content was observed in approximately 50% of the cells; half of the MeCP2 KO cell population had more L1 insertional events compared to WT cells and the maximum increase in ORF2 sequences was up to 22.8% (FIG. 10). In the other half, the number of ORF2 sequences was similar between WT and MeCP2 KO (FIG. 4B).

A control experiment was performed using individual fibroblast cells isolated from the two genetic backgrounds (FIG. 4C). No significant increase in L1 copy number was observed in MeCP2 KO when compared to WT fibroblasts (P<0.02). Moreover, fibroblasts were more homogeneous (less variation in CT values) with regard to L1 sequences when compared to WT neuroepithelial cells, highlighting the differential retrotransposition in the latter cell population (FIG. 10). As an internal control, specific primers for the L1 5′UTR were also tested in neuroepithelial cells and did not reveal a significant increase in copy number in MeCP2 KO cells (FIG. 4D). This lack of difference can be explained by the fact that, upon retrotransposition, the 5′ region of the L1 sequence is frequently truncated (6,7). No difference between genetic backgrounds was observed when using primers for non-mobile 5S ribosomal RNA repetitive sequences (FIG. 4E). Together, these observations suggest that MeCP2 mediates specific L1 retrotransposition in the mouse brain during neuronal development.

### More L1 Sequences in the Brains of Rett Syndrome Patients

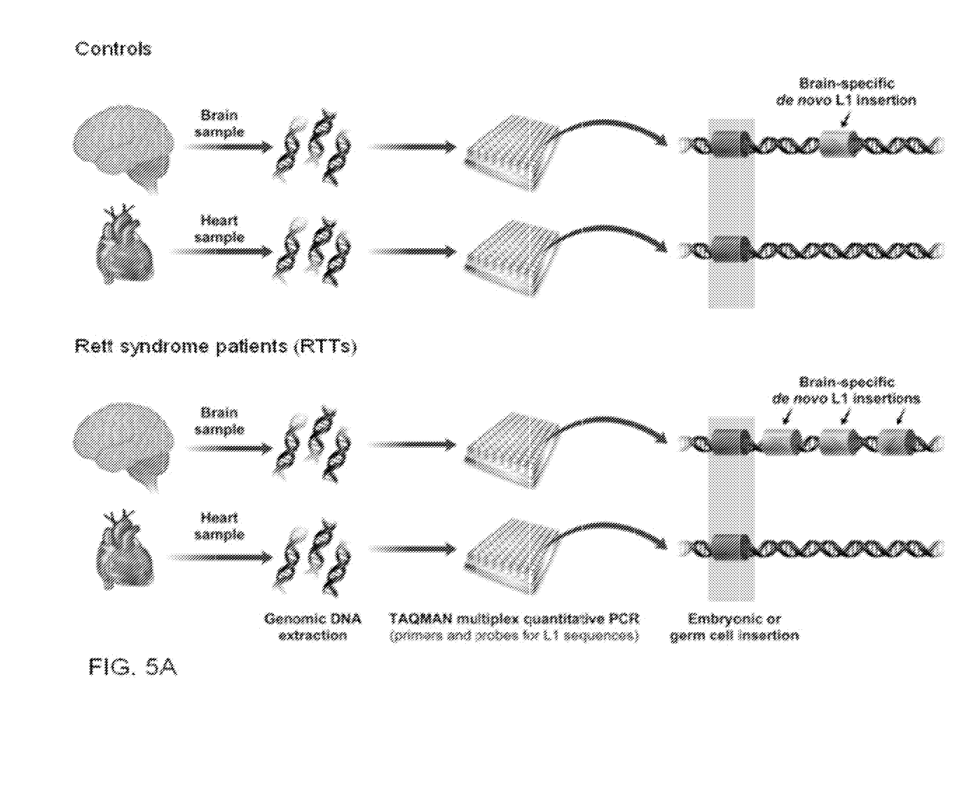

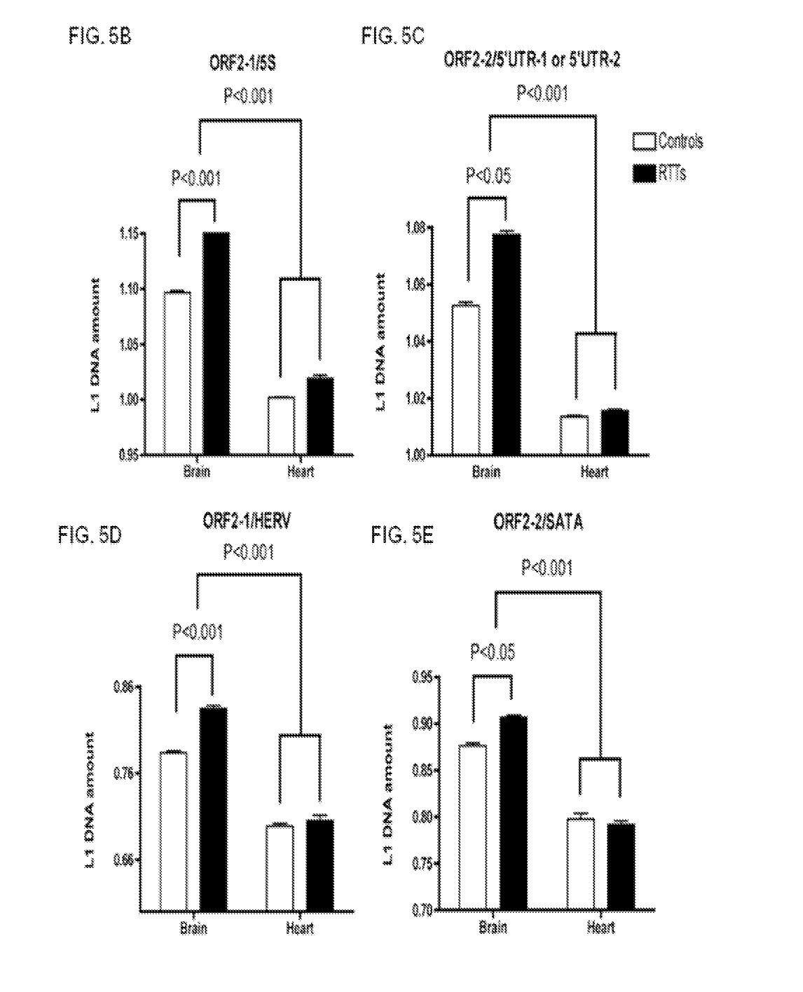

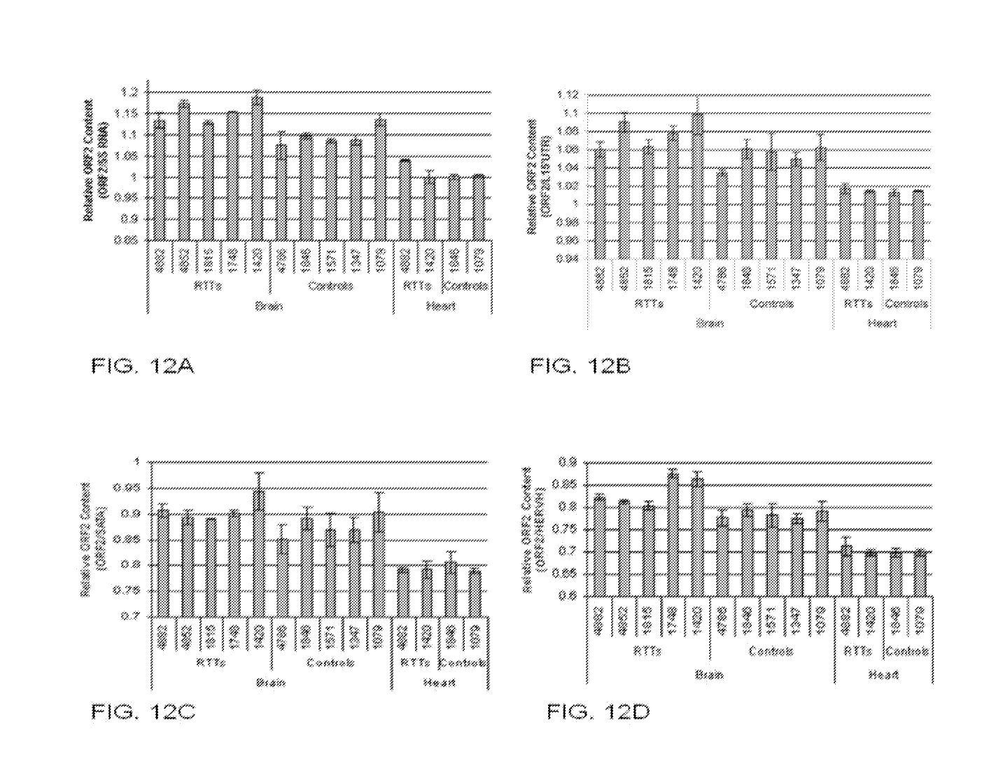

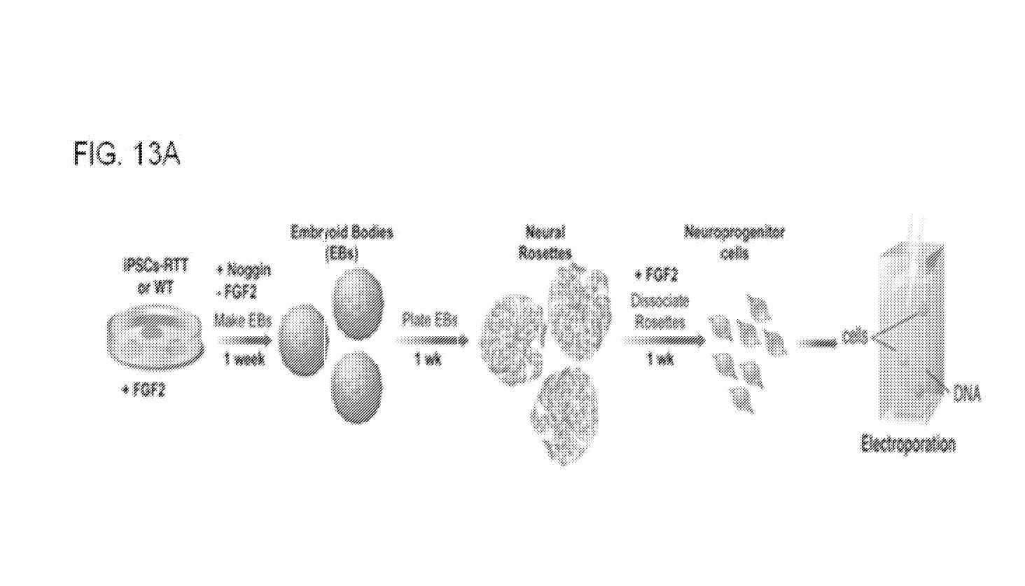

Mutations on the MeCP2 gene cause Rett Syndrome (RTT), a severe X-linked neurological disease characterized by impaired motor function; half of RTT patients develop seizures and autistic behavior at different levels of intensity (21,22). To analyze the amount of L1 retrotransposition in tissue samples with clinical diagnostics of RTT and controls, brain and other somatic tissues were obtained from the same individuals. After DNA extraction, a Taqman multiplex qPCR approach was used to compare the number of L1 ORF2 sequences normalized by non-mobile repetitive sequences in the human genome (for instance, the 5S ribosomal RNA repeats), giving a normal distribution of ORF2/5S inverted ratios (FIG. 5A). The multiplex strategy was chosen because we could not dissociate single cells from the frozen human post-mortem tissues and it is a stronger means of internally controlling DNA content within reactions. It was hypothesized that these methods would detect a higher number of L1 ORF2 sequences in the brain compared to heart due to de novo L1 neuronal retrotransposition. Moreover, based on studies in mice, it was hypothesized that the number of new insertions would be higher in brain tissues derived from RTT patients than controls. In fact, the number of L1 ORF2 sequences in the brains of RTT patients was significantly higher (P<0.001) compared to age/gender-matched controls (FIG. 5B-E). DNA quantification using controlled copies of L1 plasmid template in the qPCR reaction revealed that RTT cells have an average of 10 more L1 insertions compared to control cells (Supplementary FIG. 6). Moreover, a control somatic tissue (heart) showed no significant difference in ORF2 sequences in either RTT patients or control samples. Interestingly, in all experimental conditions, the number of ORF2 sequences was higher in brain tissues, both in controls and RTT patients, when compared to heart tissue from the same individual (FIG. 5B-E). Brain tissues also displayed a higher variation in number of ORF2 sequences compared to a more homogeneous distribution in heart (Table 1, FIG. 12). This set of experiments reveals that the data collected using the MeCP2 KO mouse can be reproduced in RTT patients and demonstrates that L1 insertions can result in brain-specific genetic alterations at an individual level.

Increased L1 Retrotransposition in NPCs Derived from iPSCs-RTT

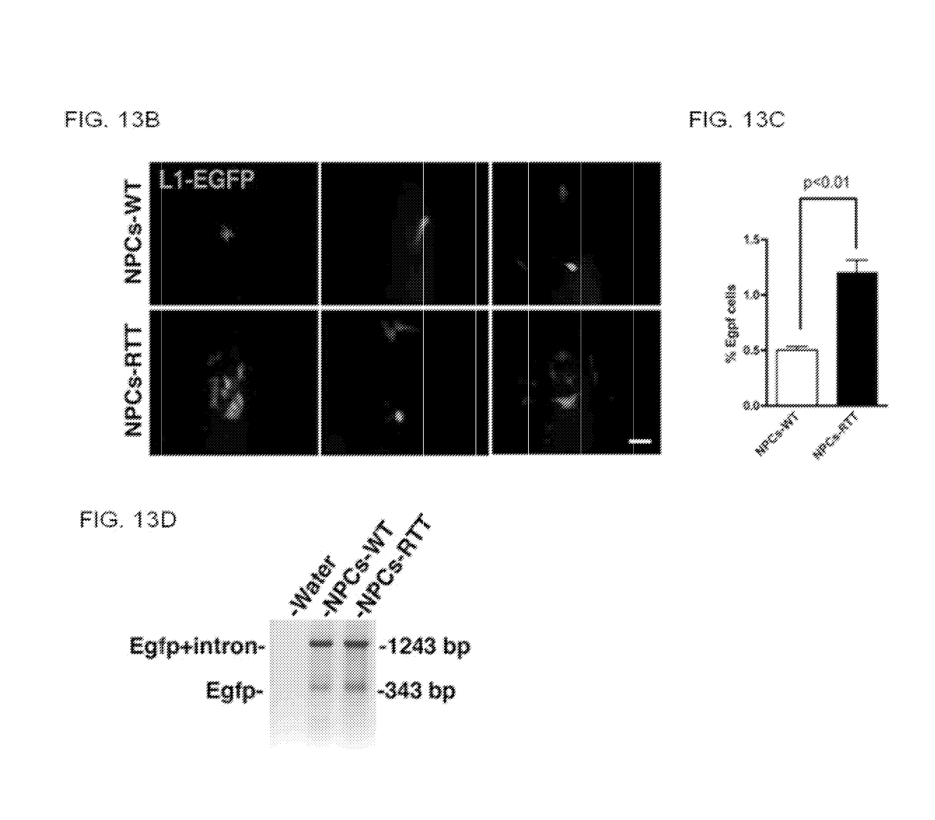

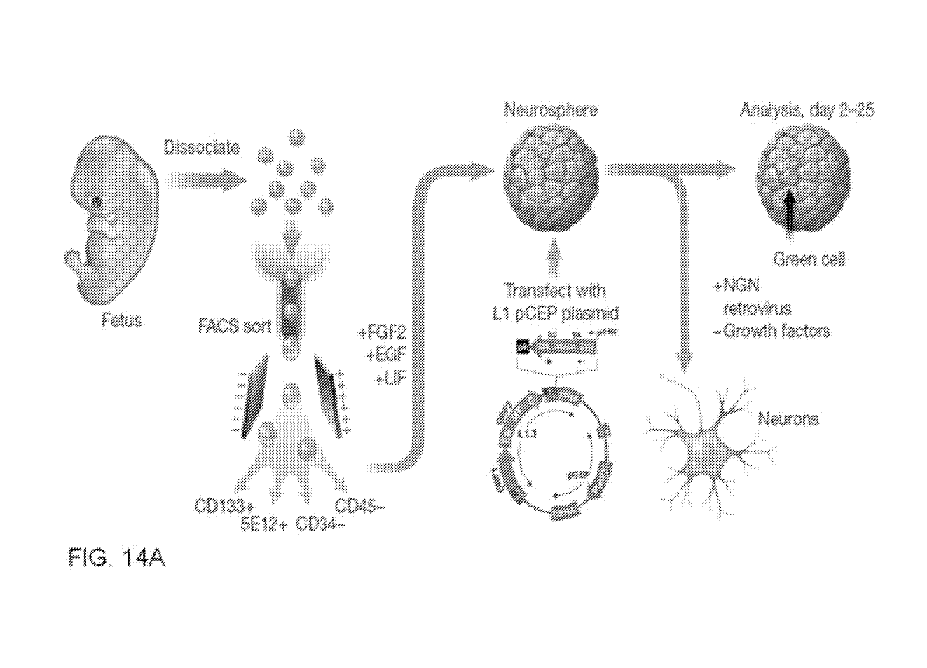

Mutations on the MeCP2 gene cause Rett Syndrome (RTT), a severe X-linked neurological disease characterized by impaired motor function; half of RTT patients develop seizures and autistic behavior at different levels of intensity (21, 22) UDATED MS. To determine if L1 retrotransposition can occur in NCPs derived from RTT patients, induced pluripotent stem cells (iPSCs) were generated from a RTT patient's fibroblasts carrying a frameshift MeCP2 mutation and from a control, non-affected healthy individual. The resultant iPSCs were isogenic to the donor cells, providing a valuable opportunity to study early stages of human development in the context of complex genetic diseases (23, 24). Clones of iPSCs derived from the RTT patient and a healthy normal individual (WT) control were pluripotent and able to generate mature, electrophysiologically active neurons in culture. Moreover, RTT-derived neurons showed reduced numbers of glutamatergic synapses and spine density as well as altered intracellular Ca²⁺ influx, indicating that the human iPSCs system can recapitulate some of the disease onset in culture. Moreover, similar to neuroepithelial cells from mice (FIG. 9A), iPSCs-derived NPCs from RTT and WT did not show differences in cell cycle. Thus, iPSCs-derived NPCs were tested to determine if they would support de novo L1 retrotransposition. Briefly, NPCs differentiation was initiated by manually isolating fragments of iPSCs colonies in suspension to form embryoid bodies (EBs) in the absence of growth factors. After a week, EBs were plated on coated dishes and neural rosettes became apparent. Dissociated rosettes formed a homogeneous population of neural NPCs that continued to proliferate in the presence of FGF-2 (FIG. 13).

NPCs were then electroporated with an active L1-element tagged with the EGFP reporter construct (L1_{RE3}-EGFP) (25, 26). EGFP expression was detected after 5-7 days in both WT and RTT-derived NPCs (FIG. 13B). The frequency of EGFP-positive cells was approximately 2-fold higher in RTT-NPCs compared to control WT NPCs (FIG. 13C). PCR confirmed the presence of the retrotransposed (i.e., spliced) EGFP gene and sequencing of the PCR products confirmed the precise splicing of the intron (FIG. 13D; data not shown). Thus, a subset of cells present in the NPC population can support L1 retrotransposition and the L1 activity is facilitated by mutations in the MeCP2 gene.

### Discussion

These data show that MeCP2, together with Sox2, is a potent suppressor of L1 expression in NSCs. Depending on the cellular context, Sox2 protein can function as an activator or repressor (23). The fact that MeCP2 is associated with Sox2 proteins confirms its repressor nature in the maintenance of NSC proliferation, adding a new factor to Sox proteins' molecular versatility. Using two different strategies, it has also been shown that L1 retrotransposition can be modulated by MeCP2 in vivo, characterizing L1 retroelements as genuine MeCP2 targets. First, it was demonstrated that L1 retrotransposition from a transgenic animal carrying an L1-EGFP indicator element was significantly higher in the brains of a MeCP2 KO genetic background compared to a WT sibling animal. Such an approach allowed visualization of de novo L1 retrotransposition in neurons. However, such approach probably underestimates the actual capacity of neuronal retrotransposition, since the engineered L1-EGFP used here represents only one of at least 3,000 active L1 elements in the mouse genome (17,18). Moreover, the L1 EGFP-indicator system does not take into account insertions that truncate or silence the reporter cassette, in trans retrotransposition of Alus or other RNAs (24-26). Second, a new technique was developed, based on single-cell genomic qPCR, to measure relative endogenous L1 sequences. The method is sensitive enough to detect differences between WT and MeCP2 KO genetic backgrounds. MeCP2 KO neuroepithelial cells, but not fibroblasts, have more L1 sequences in the genome when compared to WT cells. These results indicate that endogenous mouse L1 elements can retrotranspose during development but may be restricted to the nervous system. The qPCR data reflect a snapshot of a specific moment during brain development. If a portion of the cells that support retrotransposition survives and the rates of retrotransposition are similar during the entire development, the impact of L1 insertions may be significant, especially in the MeCP2 KO genetic background.

To study L1 retrotransposition during early stages of human development, iPSCs from a RTT patient with a MeCP2 frameshift mutation and from a normal control were derived. NPCs derived from both WT and RTT iPSCs could support L1-EGFP de novo insertions. However, RTT-NPCs showed a higher frequency of L1 retrotransposition compared to WT control cells, confirming that MeCP2 is a repressor of active human L1 retrotransposons.

A similar qPCR experiment extended these observations to human brain samples from heterozygote females RTT patients compared to normal controls and other somatic tissues. However, because mature human brain tissues, sampling from a non-homogeneous population of cells, including neurons and astrocytes, could mask more dramatic differences between neurons and non-neuronal cells. Moreover, the effects of X-chromosome inactivation status, allowing the expression of the WT MeCP2 allele only in some neurons, are likely to contribute to a subtle difference between L1 ORF2 sequences in RTT patients and controls. Finally, in the human brain, cells that survived brain development were analyzed, whereas the embryonic neuroepithelial cells isolated from the mouse will face a strong selection process wherein many cells undergo programmed cell death.

It has been hypothesized that DNA methylation and methyl-binding proteins protect the genome against retrotransposition in germ cells (27). However, the discovery that Piwi/piRNAs can suppress transposition in germ cells suggested that it may not be the only mechanism (28). The data here provides strong evidence for a role of DNA methylation-dependent MeCP2 activity in controlling transposable elements activity. Interestingly, such activity may be specific to NSC, de-repressing retroelements during neuronal differentiation, raising the question why neurons support L1 retrotransposition. Recently, re-activation of MeCP2 expression in both embryonic and adult KO mice led to prolonged life span and delayed onset or reversal of certain neurological symptoms (29,30). Since L1 insertions are genetically stable, the new insertions may have a small contribution to the reversible RTT syndrome phenotype in mouse. The high rates of neuronal retrotransposition in the MeCP2 KO mice and RTT brains may be a consequence, rather than a cause, of the disease process. However, a more effective L1 silencing may have an important impact as a modulator of neighboring gene expression. L1 sequences may function as master regulators of chromatin structure through heterochromatin silencing of discrete chromosomal regions close to neuronal genes. In that context, new somatic insertions in the MeCP2 KO mice brain and in RTT brains may contribute to the epigenetic status of neurons, affecting neuronal networks and behavior.

The data presented here are the first to demonstrate intrinsic tissue-specific somatic genetic variation outside the immune system in humans. These findings add a new layer of complexity to the understanding of genomic plasticity, revealing that neuronal genomes can accommodate somatic mutations caused by L1 retrotransposition. These observations have direct implications for genetic, non-heritable neurological diseases and individual responses to drug treatment or environmental cues.

### Example 2

Long Interspersed Element-1 (LINE-1 or L1) retrotransposons have dramatically impacted the human genome. Retrotransposons constitute approximately 40% of the mammalian genome and play an important role in genome evolution. Their prevalence in genomes reflects a delicate balance between their further expansion and the restraint imposed by the host. L1s must retrotranspose in the germ-line or during early development to ensure their evolutionary success. Yet the extent to which this process impacts somatic cells is poorly understood. It has been previously demonstrated that engineered human L1s can retrotranspose in adult rat hippocampus progenitor cells (NPCs) in vitro and in the mouse brain in vivo (34). Here it is demonstrated that NPCs isolated from human fetal brain and NPCs derived from human embryonic stem cells (hESCs) support the retrotransposition of engineered human L1s in vitro. Furthermore, a quantitative multiplex polymerase chain reaction is described that detects an increase in the copy number of endogenous L1s in the hippocampus and in several regions of adult human brains when compared to the copy number of endogenous L1s in heart or liver genomic DNAs from the same donor. The data indicate that de novo L1 retrotransposition events may occur in the human brain and, in principle, have the potential to contribute to individual somatic mosaicism.

### Methods

### Cell Culture, Transfection and Analysis

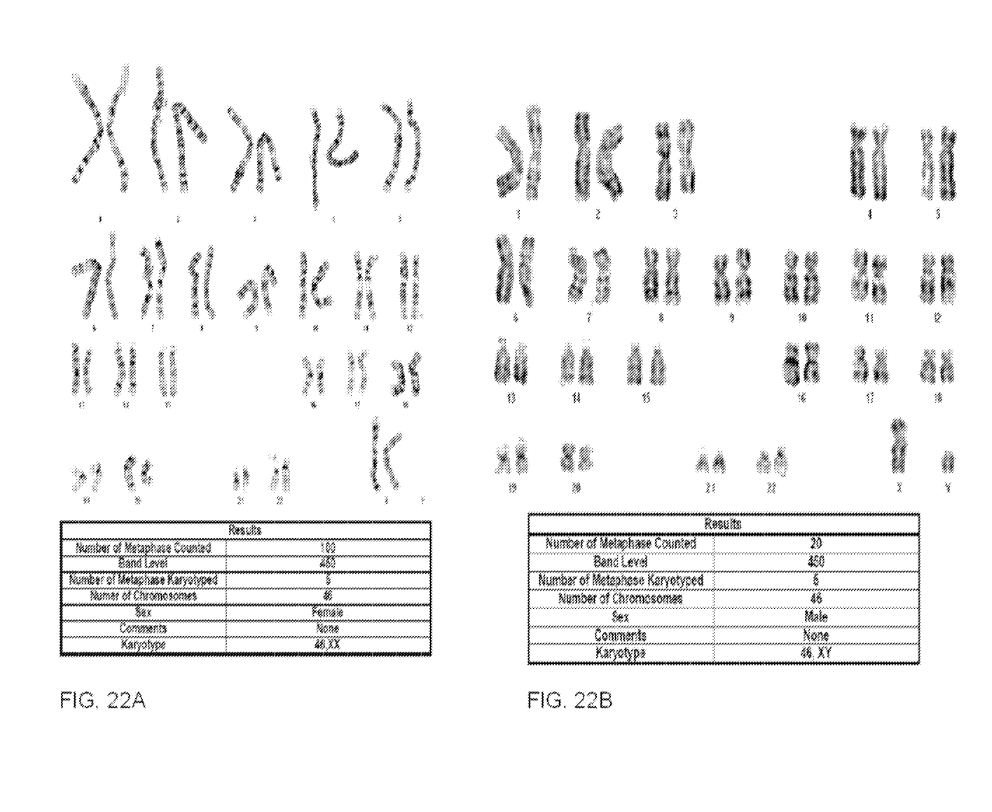

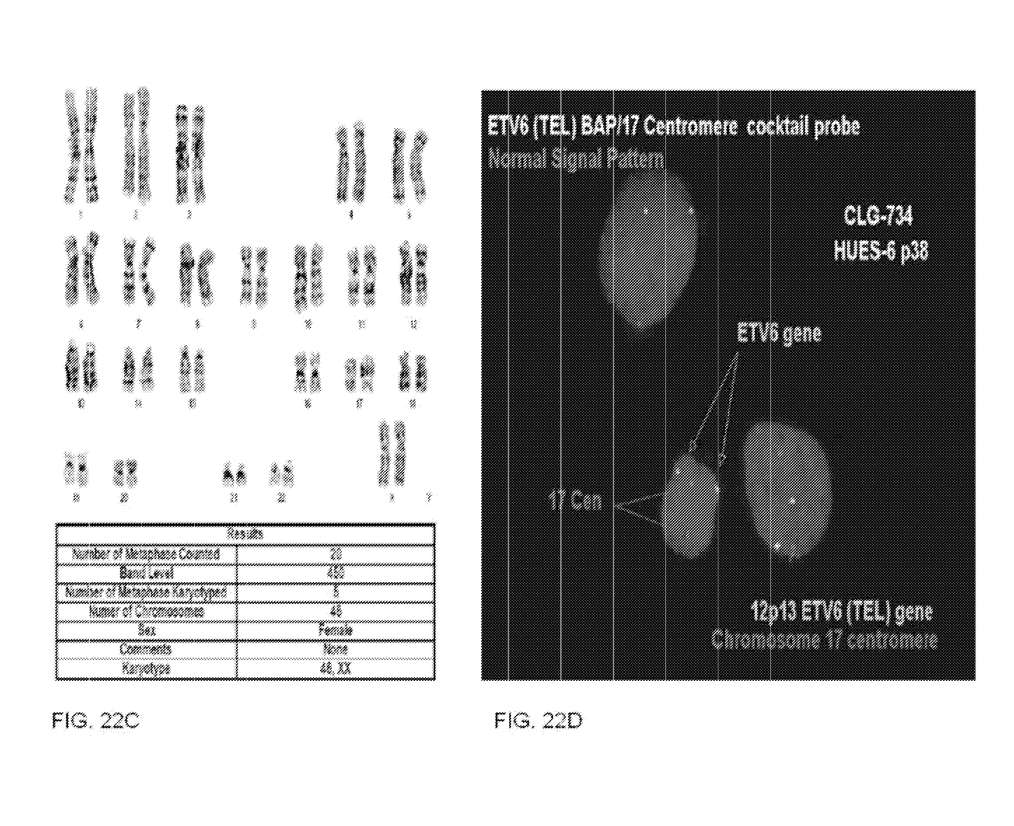

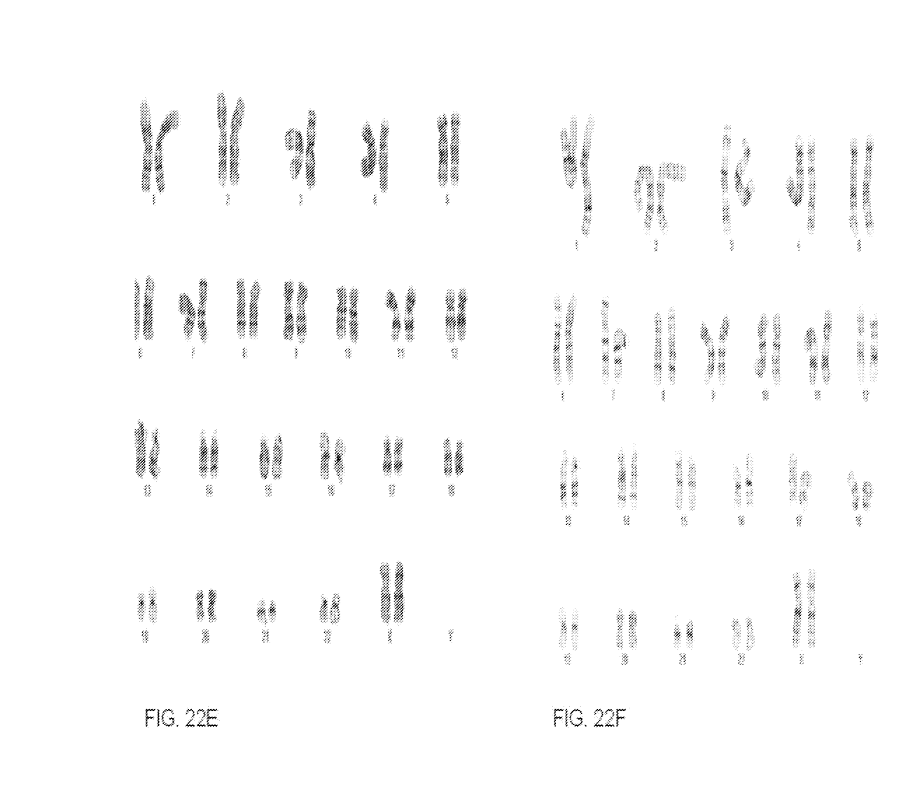

Fetal hCNS-SCns lines (36) and hESCs (57,59) were cultured as previously described. Neural progenitors were derived from hESCs as previously described (47,60). NPCs were transfected by nucleofection (Amaxa Biosystems), and either maintained as progenitors in the presence of FGF-2 or differentiated as previously described (47). Cells were transfected with L1s containing an EGFP retrotransposition cassette in pCEP4 (Invitrogen) that lacks the CMV promoter and contains a puromycin resistance gene (40). The hCNS-SCns lines FBR BR1, BR4 and BR3 were cultured as previously described and were a kind gift from Stem Cells Inc. (Palo Alto, Calif.) (36). hCNS-SCns, also known as huCNS-SC (human CNS stem cells grown as neurospheres), were derived from fetal brain by FACS using the following cell surface markers: (CD133)⁺, (5E12)⁺, (CD34)⁻, (CD45)⁻, and CD24^{-/lo}. This combination of markers enriches for progenitor neurosphere-initiating cells capable of differentiating into cells of both the neuronal and glial lineages (36). The hCNS-SCns were cultured in X-Vivo 15 media (Lonza Bioscience) supplemented with 20 ng/mL FGF-2, 20 ng/mL epidermal growth factor (EGF), 10 ng/mL leukemia inhibitor factor (LIF), N2 supplement, 0.2 mg/mL heparin, and 60 mg/mL N-acetylcysteine. For differentiation experiments, hCNS-SCns were dissociated using Liberase Blendzymes (Roche) and plated on laminin/polyornithine-coated plates. Mitogens were withdrawn and cells were differentiated by retrovirus-mediated transduction with Neurogenin 1 (NGN1), a pan-neuronal helix-loop-helix transcription factor. NGN1 was a kind gift from Dr. David Turner and was cloned into a murine Moloney leukemia retrovirus-based plasmid and expressed under the control of the ubiquitously expressed CAG promoter as previously described (63). Virus was made in human embryonic kidney 293T cells and collected by ultracentrifugation (63). hCNS-SCns were infected 48 hrs before differentiation at an approximate efficiency of 70% and allowed to differentiate for 3-4 weeks. Karyotype analysis of hCNS-SCns lines indicated grossly normal karyotype (FIGS. 22A-C).

Primary human neo-natal dermal fibroblasts (cat# CC-2509) and primary adult astrocytes (cat# CC-2565) were commercially obtained and cultured using instructions provided by the manufacturer (Lonza Bioscience). Karyotype and fluorescence in situ hybridization analyses were performed at Cell Line Genetics (Madison, Wis.). The hESC lines HUES6 and H9 were cultured as previously described (on world wide web at mcb.harvard.edu/melton/HUES/) by the Gage group (59). Under these experimental conditions, hESCs exhibited a grossly normal karyotype (FIGS. 22D-F). Briefly, cells were grown on mitomycin C-treated mouse embryonic fibroblast (MEF) feeder layers (Chemicon) in DMEM media (Invitrogen) supplemented with 20% KO serum replacement, 1 mM L-glutamine, 50 μM β-mercaptoethanol, 0.1 mM nonessential amino acids, and 10 ng/mL FGF2 (fibroblast growth factor 2). The cells were passaged by manual dissection. Karyotype analysis of hESC cells lines indicated grossly normal karyotype (FIGS. 22D-F). For embryoid body formation, cells were dissociated from the underlying MEF layer with Dispase (0.2 mg/mL; Stem Cell Technologies) and grown for 7 days in DMEM-F12 Glutamax media (Invitrogen) with N2 supplement (Gibco) and 500 ng/mL Noggin (Fitzgerald) in Petri dishes. The resulting embryoid bodies were plated onto laminin/polyornithine (Sigma)-coated plates and were grown for 7-10 days. The resultant rosettes were manually dissected and dissociated in 0.1% trypsin and plated in DMEM-F12 media supplemented with N2 and B-27, 1 μg/mL laminin, and 20 ng/mL FGF2. The resulting neural progenitors could be maintained for multiple passages prior to the induction of differentiation. Differentiation conditions involved the withdrawal of mitogens and treatment of the cells with 20 ng/ml of brain-derived neurotrophic factors (BDNF), 20 ng/ml of glia-derived neurotrophic factors (GDNF; Peprotech), 1 mM di-butyrl-cyclicAMP (Sigma), and 200 nm of ascorbic acid (Sigma) for 4-12 weeks.

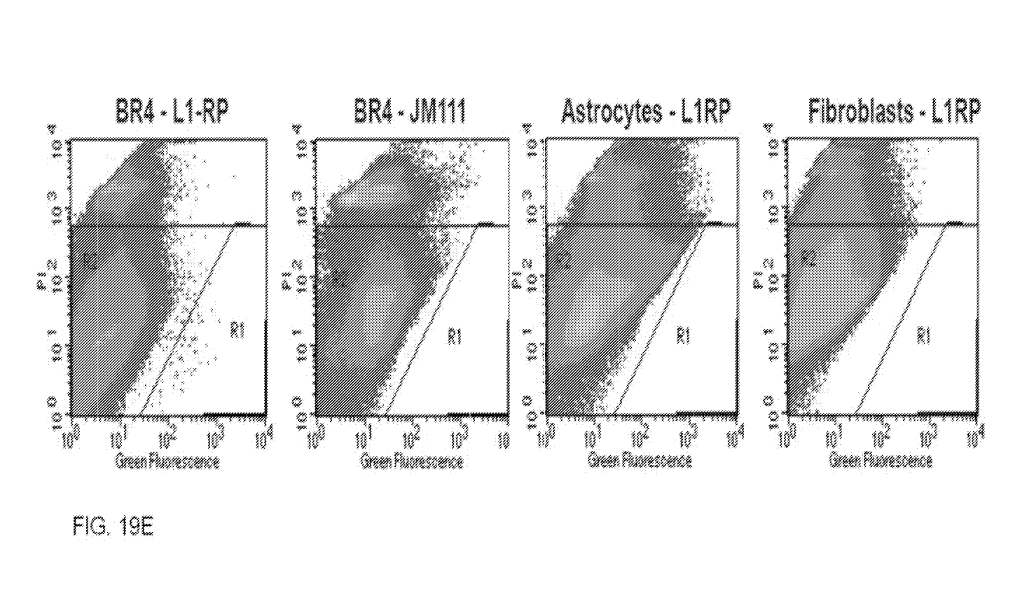

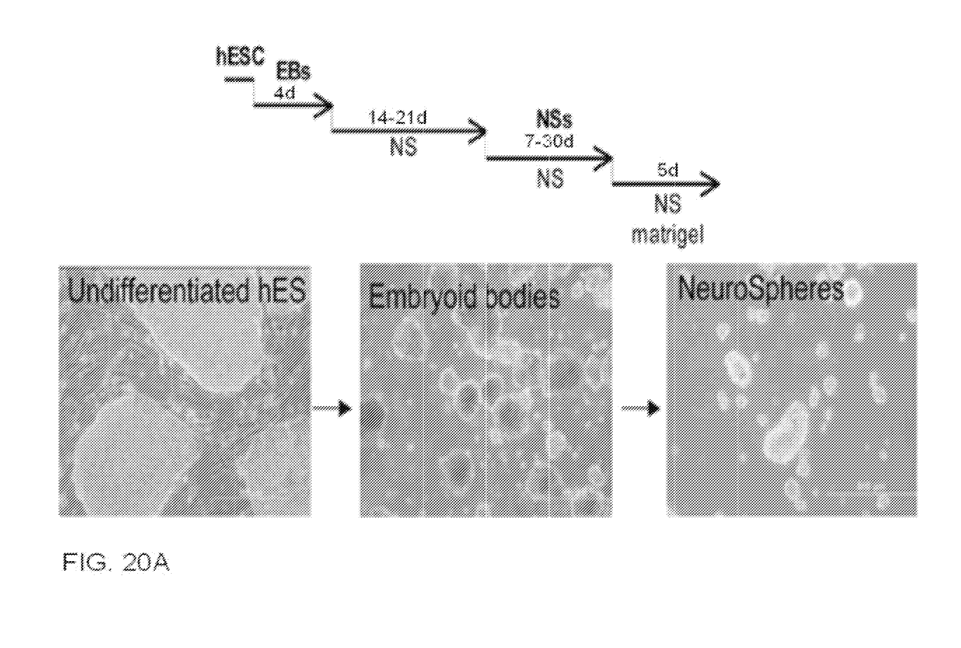

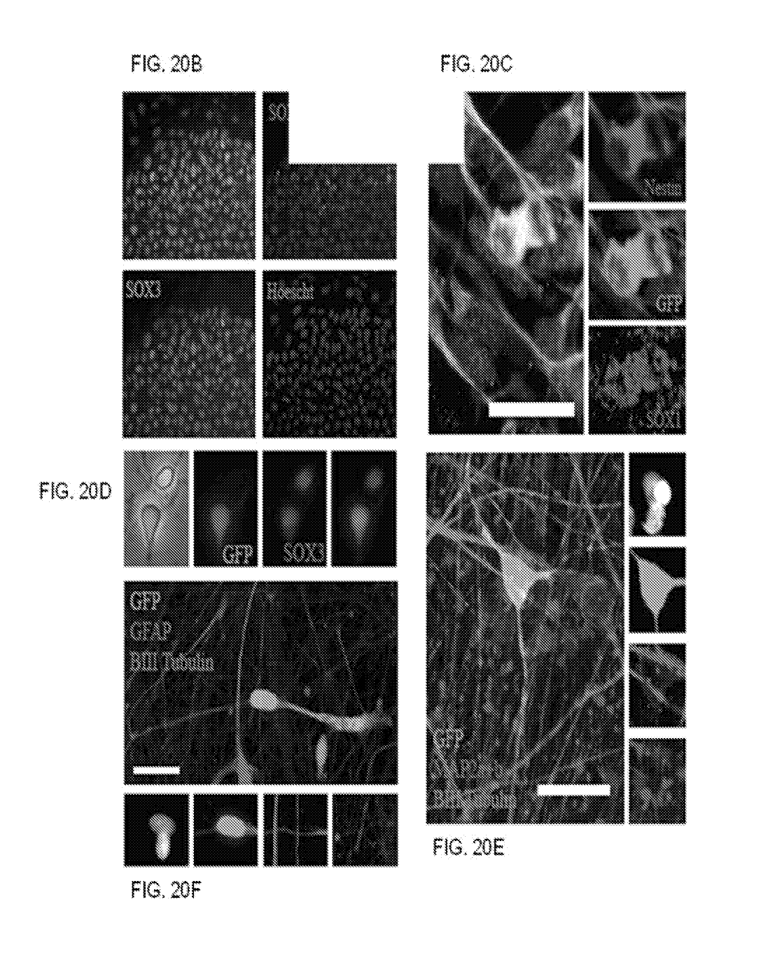

The NIH-approved hESC lines (WA07 (i.e., H7), WA09 (i.e., 119), WA13B (i.e., H13B), and BG01) were cultured as previously described by the Moran group (57). Briefly, hESCs were grown on irradiated MEFs and then were passaged by manual dissection using the StemPro EZPassage passaging tool (Invitrogen). A protocol based on Zhang et al. was used to derive NPCs (60). hESCs first were seeded in a suspension culture dish (Corning) in hESC media lacking FGF2 to generate embryoid bodies. After 4-6 days, the resulting embryoid bodies were seeded in a Petri dish coated with gelatin and cultured in NeuroSphere (NS) media for 14-16 days. NS culture medium contains DMEM F12 (Invitrogen) supplemented with 20 ng/ml FGF2, N2 supplement, and 2 μg/mL Heparin (Sigma). After 14-16 days, the resulting rosettes were picked manually, trypsinized, and then plated to form neurospheres. Neurospheres were passaged by single cell dissociation using a pulled Pasteur pipette once a week. To induce differentiation, a single cell suspension of NPCs was plated on polyornithine-coated plates in DMEM/F12 with N2 and 1% FBS and allowed to differentiate for 6 days. NPCs derived using either protocol expressed neural stem cell markers (FIGS. 20A-B).

### Constructs, Transfection, and Retrotransposition Assays

Cells were transfected with L1s containing an EGFP retrotransposition cassette in a modified version of pCEP4 (Invitrogen) that lacks the CMV promoter and contains a puromycin resistance gene instead of a hygromycin selection gene (40). Prior to transfection, DNAs were checked for superhelicity by electrophoresis on 0.7% agarose-ethidium bromide gels. Only highly supercoiled preparations of DNA (>90%) were used in transfection experiments. L1_{RP} is a full-length retrotransposition-competent L1 whose expression is driven by the native L1 5′ UTR (64,40). LRE3 is a previously described full-length retrotransposition-competent L1 (65). JM111/L1_{RP} is a derivative of L1_{RP} containing two missense mutations (RR261-262AA) in the RNA binding domain of the ORF1-encoded protein that reduce L1 retrotransposition by greater than three orders of magnitude (38,40). In UB-LRE3 and UB-JM111, the expression of the L1 is driven by the ubiquitin C promoter (a 1.2-kb fragment of the human UBC gene nucleotides 123964272-123965484 from chromosome 12). All constructs contained the CMV-EGFP retrotransposition cassette (40). The LRE3-neo and LRE3-blasticidin constructs contained the mneoI or blasticidin retrotransposition cassettes, respectively (38,44).

hCNS-SCns and HUES6- and H9-derived NPCs (one passage after neural rosette selection) were transfected by Nucleofection using the Amaxa rat NSC nucleofector solution and program A-31. The transfection efficiency was determined using an EGFP-expressing plasmid control 2 days post transfection by FACS analysis. The transfection efficiency ranged from 50-70% for hCNS-SCns and from 50-80% for hESC-derived NPCs. Cells were cultured as progenitors in the presence of mitogens. For differentiation studies, cells were dissociated and plated for differentiation 18 days after the initial transfection. H7-, H13B-, H9-, and BG01-derived NPCs were transfected using the Amaxa mouse NSC nucleofector solution and program A-33 and cultured as progenitors. In some experiments puromycin (0.2 μg/mL) was added 2 days post transfection for 5-7 days prior to scoring for retrotransposition. Primary human fibroblasts and astrocytes were transfected using Fugene6 (Roche) per manufacturer's instructions. Cells were monitored for EGFP expression by fluorescence microscopy. For FACS analysis, cells were dissociated and analyzed on a Becton-Dickinson LSR I in the presence of 1 μg/mL propidium iodide for live/dead cell gating. All assays were performed in triplicate. JM111/L1_{RP} transfected cells were used as a negative control for gating purposes. The criterion to determine L1 insertional silencing was a 10-fold increase in EGFP expression after the addition of 500 nM trichostatin-A for 16 hours on day 7 post-transfection with the L1 construct. NPCs transfected with L1s containing the mneoI or blasticidin retrotransposition indicator cassettes were subjected to either G418 or blasticidin selection beginning 4-7 days post-transfection. Cells were selected with 50 μg/ml of geneticin (G418, Invitrogen) for 1 week and with 100 μg/ml of G418 the following week, or with 2 μg/mL of blasticidin (InvivoGen) for 2 weeks.

### Electrophysiology

HUES6-derived NPCs were electroporated with the LRE3-EGFP pCEP4 plasmid, allowed to proliferate for 7 additional days, and subsequently differentiated. Whole-cell perforated patch recordings were performed on EGFP-expressing cells after 10 weeks of differentiation. The recording micropipettes (tip resistance 3-6 MΩ) were tip-filled with internal solution composed of 115 mM K-gluconate, 4 mM NaCl, 1.5 mM MgCl₂, 20 mM HEPES, and 0.5 mM EGTA (pH 7.4) and then back-filled with the same internal solution containing 200 μg/ml amphotericin B. Recordings were made using Axopatch 200B amplifier (Axon Instruments). Signals were sampled and filtered at 10 kHz and 2 kHz, respectively. The whole-cell capacitance was fully compensated, whereas the series resistance was uncompensated but monitored during the experiment by the amplitude of the capacitive current in response to a 5-mV pulse. The bath was constantly perfused with fresh HEPES-buffered saline composed of 115 mM NaCl, 2 mM KCl, 10 mM HEPES, 3 mM CaCl₂, 10 mM glucose and 1.5 mM MgCl₂ (pH 7.4). For current-clamp recordings, cells were clamped at ˜−60 to −80 mV. For voltage-clamp recordings, cells were clamped at −70 mV. All recordings were performed at room temperature. Amphotericin B was purchased from Calbiochem. All other chemicals were from Sigma.

### Immunocytochemistry and Imaging

Cells were fixed in 4% paraformaldehyde, and immunocytochemistry was performed as previously described (57,66). Antibodies and dilutions were as follows: βIII tubulin, mouse monoclonal, 1:400 or rabbit polyclonal, 1:500 (both Babco/Covance); Map (2a+2b), mouse monoclonal, 1:500 (Sigma); GFAP rabbit polycolonal, 1:300 (DAKO); GFAP, guinea pig polyclonal, 1:1000 (Advanced Immunochemical); Nestin, mouse monoclonal, 1:800 (Chemicon); Musashi-1, rabbit polyclonal, 1:200 (Chemicon); Sox1, rabbit polyclonal, 1:200 (Chemicon); Sox1, goat polyclonal, 1:200 (R&D); TH, rabbit polyclonal, 1:500 (Pel-Freez); Ki-67, rabbit monoclonal, 1:500 (VectorLabs); Sox2, rabbit polyclonal, 1:500 (Sigma), Sox3, rabbit polyclonal, 1:500 (a generous gift from Dr. M. W. Klymkowsky, Denver, Colo.). Secondary antibodies were purchased from Jackson ImmunoResearch or Invitrogen and all were used at 1:250. Cells were imaged using a CARVII spinning disk confocal imaging system (BD).

### Luciferase Assays

Luciferase activity was measured with the Dual-Luciferase reporter assay system according to instructions provided by the manufacturer (Promega). In all assays, a plasmid expressing the Renilla luciferase gene was used as an internal control. The assays were replicated independently at least three times. The L1 5′ UTR luciferase construct has been previously described (34,67). The Synapsin-1 promoter region was a kind gift from G. Thiel. All promoters were subcloned into the pGL3-basic vector (Promega).

### Southern Blot

Southern blotting was performed following standard protocols (68) on hCNS-SCns line FBR4 collected 3 months post-transfection with the L1_{RP} pCEP4 plasmid. Briefly, 20 μg of genomic DNA was digested with CM, a restriction enzyme that digests the tagged-L1 both at 5980 bp (20 bp 5′ to start of the retrotransposition cassette) and at 8517 bp (in the 3′ UTR). The L1.3 plasmid containing the indicator cassette yields a 2547 bp band, whereas a retrotransposed L1 integrated into a genomic sequence that lacks the intron in the EGFP expression cassette yields a 1645 bp band. This methodology collapses all the tagged L1 insertions into a single imaged band. The probe was a full-length EGFP DNA fragment that was radioactively labeled with γ-³²P-dCTP using the Random Prime Labeling Kit according to instructions provided by the manufacturer (Roche).

### Cell Lysates and Western Blot Analysis

hESC or NPCs were harvested and lysed as previously described with 1 ml of 1.5 mM KCl, 2.5 mM MgCl₂, 5 mM Tris-Hcl pH 7.4, 1% deoxycolic acid, 1% Triton X-100, and 1× Complete Mini EDTA-free Protease Inhibitor cocktail (Roche) (41). Cell debris was removed by centrifugation at 3,000×g at 4° C. for 5 minutes, and 10% of the supernatant fraction was saved (i.e., Whole Cell Lysate or WCL fraction). A sucrose cushion then was prepared with 8.5% and 17% w/v sucrose in 80 mM NaCl, 5 mM MgCl₂, 20 mM Tris-Hcl pH 7.5, and 1 mM DTT, which was supplemented with 1× Complete Mini EDTA-free Protease Inhibitor cocktail (Roche). WCLs were centrifuged at 39,000 rpm for 2 hours at 4° C. using a Sorvall SW-41 rotor. After centrifugation, the pelleted material (i.e., the ribonucleoprotein particle (RNP) sample) was resuspended in 50 μL of purified water supplemented with 1× Complete Mini EDTA-free Protease Inhibitor cocktail (Roche). Total protein concentration was determined by Bradford assay according to instructions provided by the manufacturer (BioRad). WCL and/or RNP samples (8 μg of each sample) were loaded on 10% SDS-PAGE gels (BioRad). Antibodies and dilutions were as follows: Anti-ORF1, rabbit polyclonal antibody, 1:10,000 dilution (a generous gift from Dr. Thomas Fanning); anti-S6 ribosomal protein, rabbit polyclonal antibody, 1:1,000 dilution (Cell Signaling); anti-Sox3 antibody, rabbit polyclonal, 1:1,000 dilution (a generous gift from Dr. M. Klymkowsky); anti-Sox1, goat polyclonal, 1:500 dilution (R&D). All HRP conjugated secondary antibodies were used at a 1:20,000 dilution (abeam).

### Cell Lysates

Ribonucleoprotein particles were isolated and analyzed as previously described (41). Luciferase assays were performed as previously described (34). Chromatin immunoprecipitation was performed utilizing primers towards the L1 5′UTR and a ChIP assay kit (Upstate/Millipore) as per manufacturer's protocol.

### RNA

RNA was isolated from various cell and tissue types with RNABee (Tel-test Inc., Friendswood Tex.) following the manufacturer's directions. RNA quality was verified by gel electrophoresis, and cDNA was synthesized using the cells-to-cDNA H kit (Ambion/Applied Biosystems) per manufacturer's instructions. Quantitative RT-PCR was performed with the same ORF2 #1 Taqman primer/probe combination utilized for genomic DNA analysis. Standardization was performed using the beta-actin Taqman Detection Kit (Applied Biosystems). RT-PCR analysis was performed using the following primers towards ORF1:

ORF1-Fw:
(SEQ ID NO: 32)
5′-GCTGGATATGAAATTCTGGGTTGA
ORF1-Fw:
(SEQ ID NO: 33)
5′-GCTGGATATGAAATTCTGGGTTGA

and PCR products from RT-PCR and QPCR reactions were cloned into the PCR TOPO II vector (Invitrogen) and sequenced.

### Bisulfite Analysis

Fetal tissues were obtained from the Birth Defects Research Lab at the Univ. of Washington. Bisulfite conversions were performed by manufacturer's instructions utilizing the Epitect kit (Quiagen). BLASTN (available on the Internet at blast.ncbi.nlm.nih.gov/Blast.egi) was used to align sequences to a database of full-length L1s. Fetal tissues were obtained from donations resulting from voluntary pregnancy terminations and were collected by the Birth Defects Research Lab at the University of Washington, Seattle, Wash. (NIH HD 000836). Genomic DNAs from 80-day-old female and 82-day-old male fetuses were isolated from brain and skin tissue using standard phenol-chloroform extraction techniques. The resulting DNA was digested with the restriction enzyme DraI and the bisulfite conversion reaction was performed using the Epitect kit according to instructions provided by the manufacturer (Qiagen). The bisulfite conversion was performed two times, consecutively, to achieve a CpG conversion rate of >90% in the LINE-1 repeat regions. The L1 5′ UTR contains a CpG island that has a G+C content greater than 60% and a CpG frequency ratio of greater than 0.6 (observed/expected CpGs) (16). The sequence of all full-length Ta-subfamily L1s was used to design oligonucleotide primers that allowed us to amplify a 363 bp region from a constellation of L1 s, which included both young Ta-1 and older subfamilies of the L1Hs/L1PA1 family such as Ta-0 due to the high degree of L1 sequence conservation (42,43). Thus, the following primers were designed against sequences in the L1 5′ UTR using Methyl Primer Express:

For:
(SEQ ID NO: 34)
5′-AAGGGGTTAGGGAGTTTTTTT
Rev:
(SEQ ID NO: 35)
5′-TATCTATACCCTACCCCCAAAA

and the resulting PCR products were cloned into the TOPO TA 2.1 plasmid (Invitrogen) and 100 bacterial colonies were sequenced from each tissue sample.

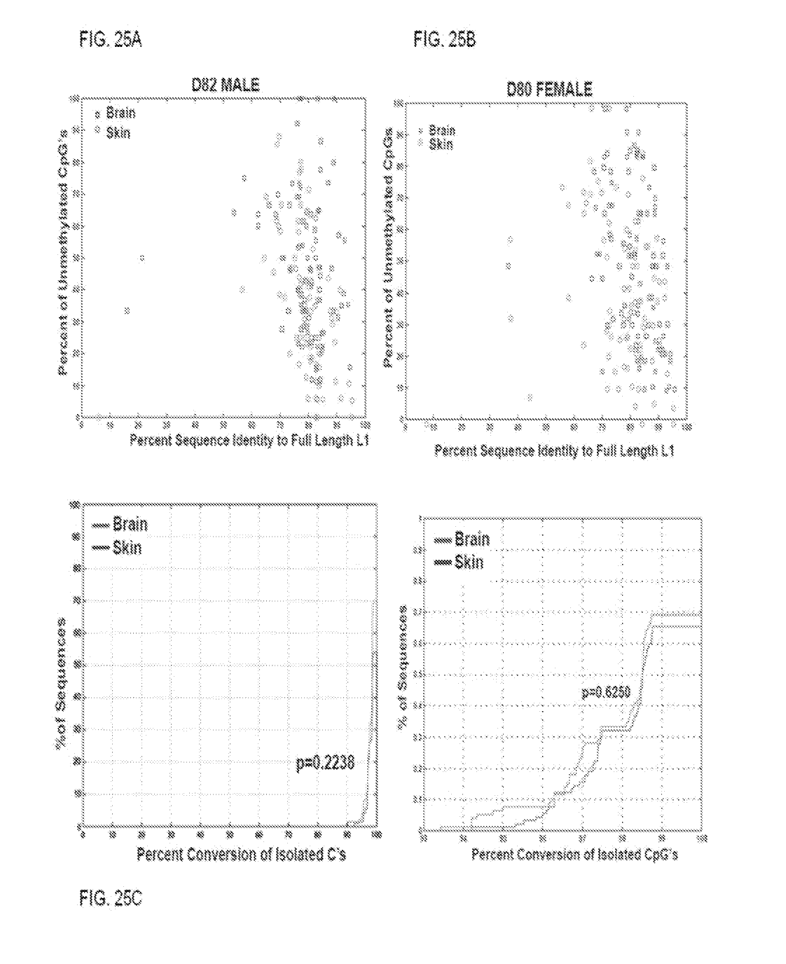

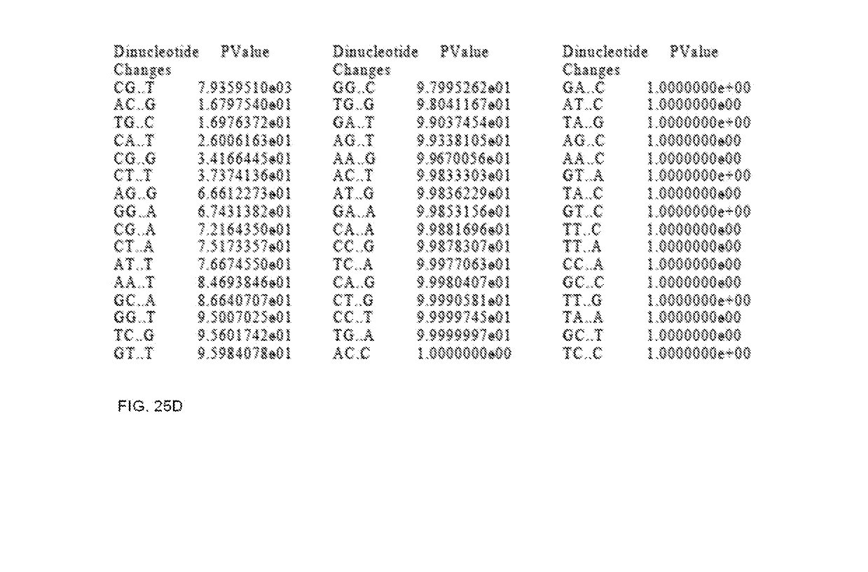

After bisulfite treatment, BLASTN was used to align the L1 5′ UTR sequences to a database of full-length L1s with two intact open reading frames that was extracted from the May 2004 assembly of the human genome (hg17). The BLASTN alignment used a mismatch penalty of −1 and a match reward of +1. The best match for each brain or skin sequence to the genomic L1 database was determined (the database consisted of known RC-L1s). The alignment excluded cytosine nucleotides in the L1 database to prevent bias due to the bisulfite conversion. The fraction of CpG sites that were unmethylated was calculated by computationally comparing CpG dinucleotides in the L1 database to the corresponding sequences from the brain and skin samples. The fraction of CpG sites converted by the bisulfite analyses was measured as the proportion of TG dinucleotides in brain and skin sequences at CpG sites in the genomic L1 database to total number of CpG sites in the region. To determine differences in methylation between brain and skin L1 s, a cumulative distribution (CDF) plot was generated for all the sequences that aligned above an alignment cutoff. The alignment cutoff was one standard deviation below the mean of the alignment identity score for all sequences aligned. Conversion efficiency was assessed by analyzing the conversion rate at genomic cytosine nucleotides that were not upstream of a guanine nucleotide. The same analysis was carried out for all possible dinucleotides and possible conversions of the first nucleotide. A two-sample Kolmogorov-Smirnov test indicated a statistically significant difference between skin and brain. Comparison of each dinucleotide pair within each sequence revealed a statistically significant difference in the CpG bisulfite conversion efficiency (i.e., CpG to TpG nucleotide changes) between the brain and skin samples but not in any of the other dinucleotide pairs in the L1 5′ UTR (FIG. 25D). Significance was only reached by the conversion of cytosines at CpG sites in brain as compared to skin samples; all other dinucleotide pairs were not statistically significant.

### Chromatin Immunoprecipitation

Chromatin immunoprecipitation (ChIP) was performed following the manufacturer's protocol and a ChIP assay kit (Millipore/Upstate). The protocol was modified such that antibody hybridization was performed twice to decrease background. Antibodies used were anti-Sox2 (Chemicon), anti-MeCP2 (Chemicon), and IgG. Resultant purified DNA was amplified with the following primers designed to the active LRE3 element (64,65), amplifying the SOX2 binding sites:

L1-Sox2-Fw
(SEQ ID NO: 36)
5′-AGATCAAACTGCAAGGCGGCAAC
L1-Sox2-Rv
(SEQ ID NO: 37)
5′-TCTTCAAAGCTGTCAGACAGGGACAC;

and towards the CpG island region:

L1-CpG-Fw:
(SEQ ID NO: 38)
5′-AATAGGAACAGCTCCGGTCTACAGCTCC
L1-CpG-Rv:
(SEQ ID NO: 39)
5′-CGCCGTTTCTTAAGCCGGTCTGAAAAG;

the Sox2 primers were used with ChIP utilizing antibodies towards SOX2, and primers designed towards the CpG island were utilized with MeCP2 immunoprecipitated DNA.

### PCR

Adult human tissues were obtained from the NICDH Brain and Tissue Bank for Developmental Disorders (University of Maryland, Baltimore, Md.). Taqman probes and primers were designed using L1 Base (on Internet at llbase.molgen.mpg.de/) and copy number estimates were based on the UCSC genome browser (on Internet at genome.ucsc.edu). Experiments were performed on an ABI Prism 7000 sequence detection system (Applied Biosystems). For each tissue, three separate tissue samples were extracted and considered as repeated measures. Whole genome size was estimated based on the equation, cell genomic DNA content=3*10⁹(#bps)*2(diploid)*660 (MW 1 bp)*1.67*10¹² (weight 1 dalton), resulting in the approximation that one cell contains 6.6 pg genomic DNA (61). Therefore, the 80 pg of genomic DNA utilized per reaction is derived from approximately 12 cells. Inverse PCR was performed as previously described (34,57). Genomic DNA from transfected NPCs and hCNS-SCns was isolated using the DNeasy Blood & Tissue kit according to instructions provided by the manufacturer (Qiagen). Genomic DNA was collected 8 days post-transfection from NPCs and 2 months post-transfection from hCNS-SCns. To assay for removal of the intron from the retrotransposition indicator cassette, 200 ng of genomic DNA was used in a 25 μL PCR reaction with the following primers: EGFP968s and EGFP1013 as (in experiments conducted with EGFP-tagged L1s), NEO437s and NEO1808 as (in experiments conducted with mneoI-tagged L1s), or Blast-Fw (5′-GCTGTCCATCACTGTCCTTCA (SEQ ID NO:40)) and Rv (5′-CCATCTCTGAAGACTACAGCG (SEQ ID NO:41)) primers (in experiments conducted with blasticidin-tagged L1s). PCR cycling conditions were described previously, and the blasticidin cycling conditions were identical to those utilized for the mneoI PCR (38,40). The marker ladder utilized in all gel pictures is a 1 kB plus ladder (Invitrogen, catalog#10787-081). Sequence analysis of all L1 PCR products was performed with the USCS genome browser (on world wide web at genome.ucsc.edu) and Repeatmasker (on world wide web at repeatmasker.org).

### Insertion Characterization by Inverse PCR

Initially fluorescent activated cell sorting (FACS) was used to isolate EGFP-positive and EGFP-negative NPCs 18 days post-transfection. However, no clones grew to confluence in a 96-well plate, and after whole genome amplification and inverse PCR (34,44,57) only a single retrotransposition event was characterized (Table 3). Single EGFP-positive cells were sorted into 96-well plates and allowed to proliferate for 6-8 weeks. Cells then were trypsinized using 10 mL of Tryple reagent (Invitrogen). Since the DNA yield for the single colonies was very low, whole genome amplification was performed using the Genomiphi kit according to instructions provided by the manufacturer (GE Life Sciences).

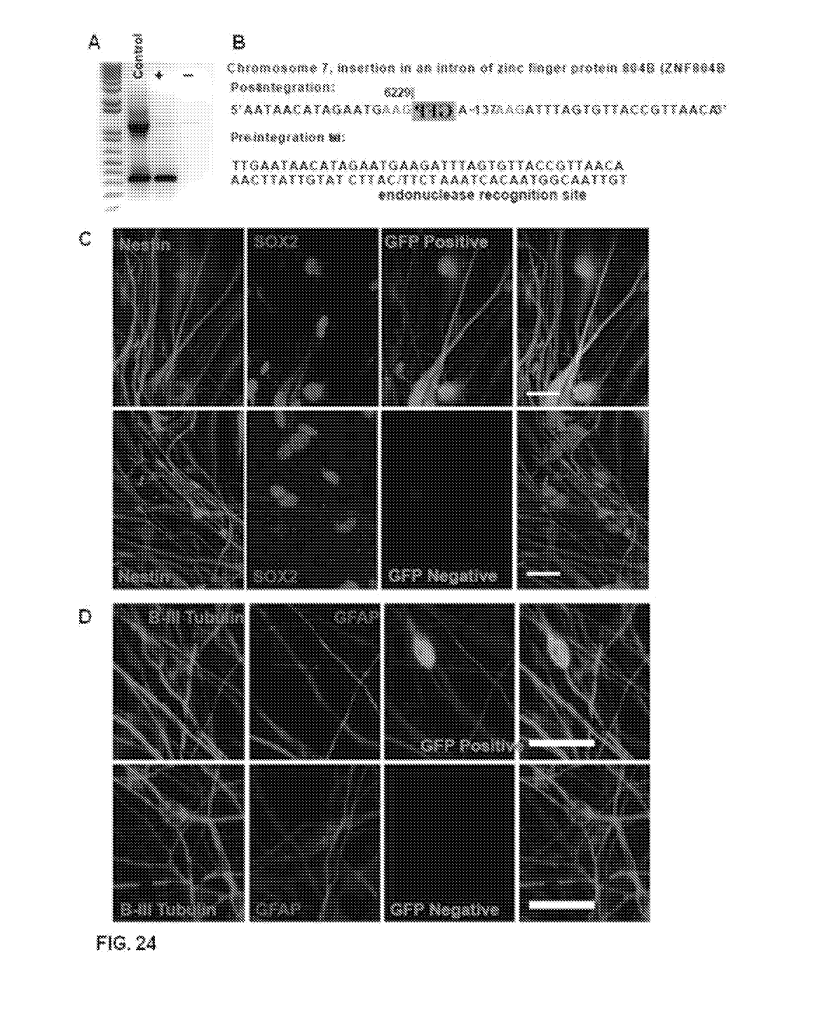

Cells harboring retrotransposition events derived from native, full-length, LRE3-tagged element insertions were grown in NPC medium for 18-20 days. The resulting cells were dissociated with trypsin and sorted on a Becton-Dickenson FACscan. A total of 40,000 EGFP-positive cells and EGFP-negative cells was sorted and expanded in culture for three passages. This experiment was replicated independently using a second sample of independently derived NPCs. As expected, genomic DNA from the EGFP-positive cells yielded a PCR product corresponding to the retrotransposed EGFP gene (FIG. 24A). These cells proliferated in culture and, like the EGFP-negative control cultures, expressed the expected neural stem markers (FIG. 24C), and could be differentiated to neuronal and glial lineages at similar rates to control cultures (FIG. 24D). The cells were harvested and genomic DNA was isolated using standard phenol-chloroform techniques.

Inverse PCR (IPCR) was performed as previously described (34,57). Briefly, 5-10 μg of genomic DNA was digested overnight with either SspI or XbaI. The digested DNA then was ligated under dilute conditions in a final volume of 1 mL with 3,200 U of T4 DNA ligase (NEB) overnight at 4° C. The circular ligated DNA was concentrated to 50 μL using a Microcon 100 column (Millipore), and then was subjected to IPCR using previously described conditions (34,57). PCR products were gel-isolated, cloned into the TOPO TA 2.1 plasmid (Invitrogen) and sequenced. Identification of the L1 pre-integration sites and other DNA sequence analyses were performed using the UCSC genome browser (March 2006 assembly) (69).

### Quantitative PCR

Oligonucleotide PCR primers were purchased from Allele Biotech and TaqMan-MGB probes from Applied Biosystems and were designed using Primer Express software (Applied Biosystems). L1 primers were verified using the L1 database L1 Base and matched a minimum of 140 of 145 full-length L1s with two intact open reading frames in the database. Human tissues were obtained from the NICDH Brain and Tissue Bank for Developmental Disorders (University of Maryland, Baltimore, Md.). Donors were between 17 and 45 years old. Dissection of the subventricular zone (SVZ), dentate gyrus (DG), CA1 and CA3 regions was performed from human brain sections. Human genomic DNAs were extracted and purified from tissues using the DNeasy Blood & Tissue kit according to instructions provided by the manufacturer (Qiagen). PCR reactions were carried out using 80 pg of genomic DNA and were verified empirically as amplifying with a cycle threshold (CT) value between 20 and 25 (n=16). Whole genome size was estimated based on the equation, cell genomic DNA content=3*10⁹(#bps)*2(diploid)*660 (MW 1 bp)*1.67*10¹² (weight 1 dalton), resulting in the approximation that one cell contains roughly 6.6 pg genomic DNA (61).

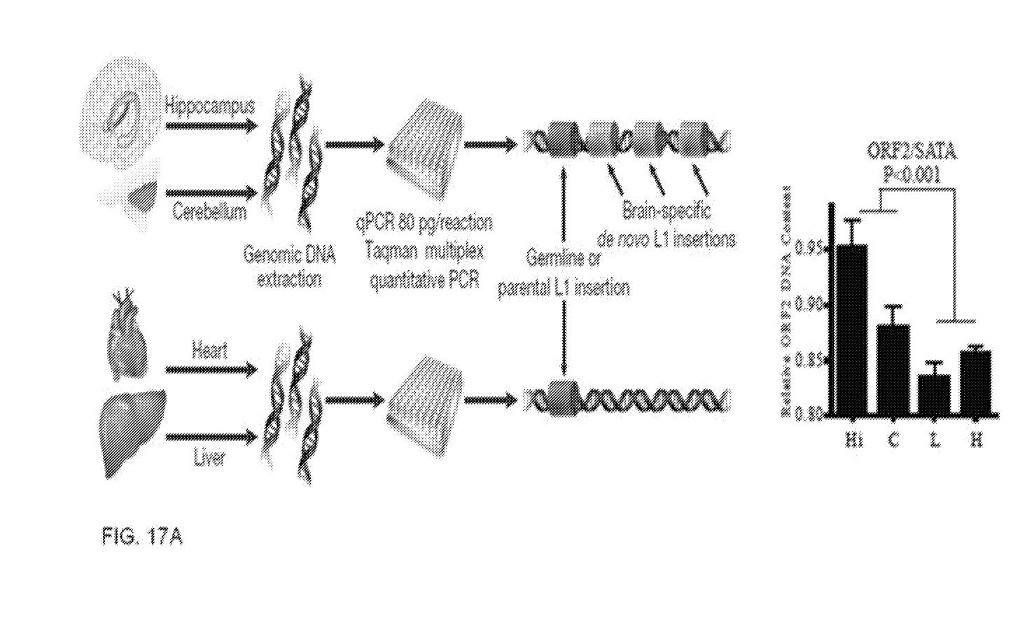

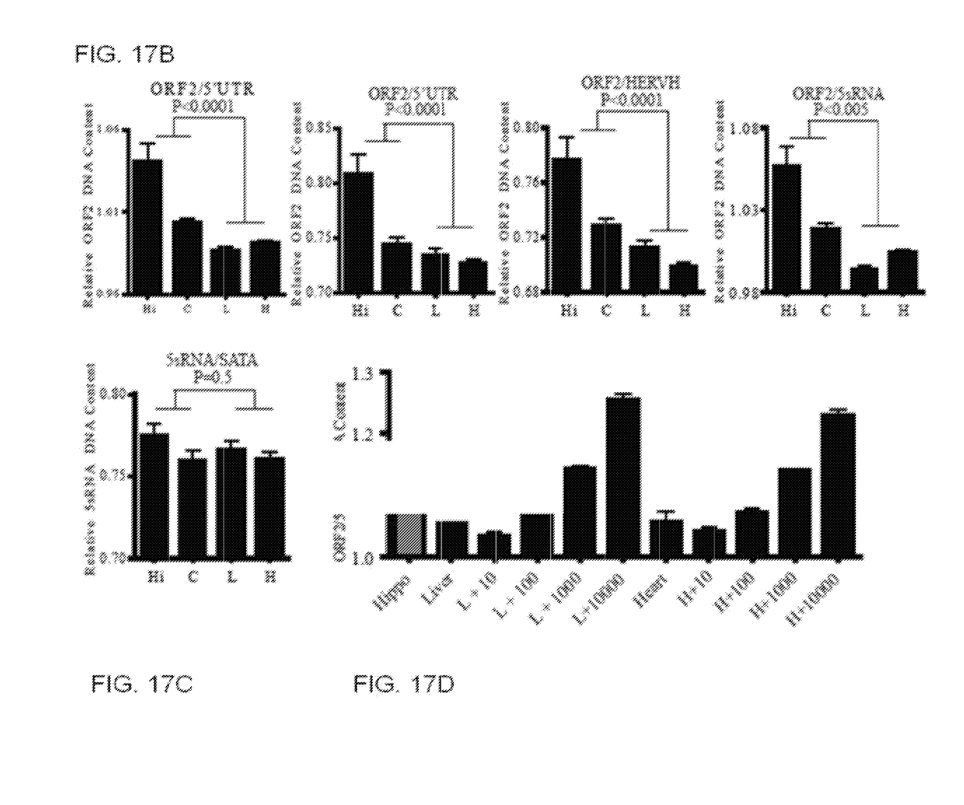

Quantitative PCR experiments were performed using an ABI Prism 7000 sequence detection system and Taqman Gene Expression Mastermix (Applied Biosystems). Data analysis was performed with SDS 2.3 software (Applied Biosystems). The multiplexing reaction was optimized by limiting reaction components until both reactions amplified as well as each individual reaction. Standard curves of genomic DNA ranging from 2 ng to 16 pg were performed to verify the 80 pg dilution used is within the linear range of the reaction. Primer efficiency and multiplexing effectiveness was verified by linear regression to the standard curve and indicated a slope near −3.32, representing acceptable amplification of both PCR products and matched primer efficiencies. ORF2 probes were conjugated to the fluorophore label VIC and all other probes were conjugated with 6FAM. For the control assay depicted in FIG. 17E (5S rDNA/SATA), the 5S rDNA probe was generated with the VIC fluorophore in order to multiplex with the SATA-6FAM probe set. For each assay, the ratio of ORF2 to control probe was normalized to 1.0 for the lowest liver value, and all other samples were normalized relative to this lowest liver value. Primers and probes are listed below; copy numbers were determined using the UCSC genome browser in silico PCR function:

L1 ORF2 #1: Matches 4,560 genomic L1s:
Probe:
(SEQ ID NO: 42)
5′-CTGTAAACTAGTTCAACCATT;
For:
(SEQ ID NO: 43)
5′-TGCGGAGAAATAGGAACACTTTT;
Rev:
(SEQ ID NO: 13)
5′-TGAGGAATCGCCACACTGACT;
L1 ORF2 #2: Matches 2,918 genomic L1s:
Probe:
(SEQ ID NO: 14)
5′-AGGTGGGAATTGAAC;
For:
(SEQ ID NO: 15)
5′-CAAACACCGCATATTCTCACTCA;
Rev:
(SEQ ID NO: 16)
5′-CTTCCTGTGTCCATGTGATCTCA;
L1 5′ UTR #1: Matches 965 genomic L1s:
Probe:
(SEQ ID NO: 17)
5′-AAGGCTTCAGACGATC;
For:
(SEQ ID NO: 18)
5′-GAATGATTTTGACGAGCTGAGAGAA;
Rev:
(SEQ ID NO: 19)
5′-GTCCTCCCGTAGCTCAGAGTAATT;
L1 5′ UTR #2: Matches 876 genomic L1s:
Probe:
(SEQ ID NO: 20)
5′-TCCCAGCACGCAGC;
For:
(SEQ ID NO: 21)
5′-ACAGCTTTGAAGAGAGCAGTGGTT;
Rev:
(SEQ ID NO: 22)
5′-AGTCTGCCCGTTCTCAGATCT;
SATA: Matches the myriad of a-satellite tandem
copies in genome, little sequence variability,
perhaps millions of copies (52):
Probe:
(SEQ ID NO: 23)
5′-TCTTCGTTTCAAAACTAG;
For:
(SEQ ID NO: 24)
5′-GGTCAATGGCAGAAAAGGAAAT;
Rev:
(SEQ ID NO: 25)
5′-CGCAGTTTGTGGGAATGATTC;
5S rDNA gene: Matches the 5S rDNA genes in the
genome, approximately 35 copies:
Probe:
(SEQ ID NO: 26)
5′-AGGGTCGGGCCTGG;
For:
(SEQ ID NO: 27)
5′-CTCGTCTGATCTCGGAAGCTAAG;
Rev:
(SEQ ID NO: 28)
5′-GCGGTCTCCCATCCAAGTAC;
HERV-H: Matches 99 copies in the genome:
Probe:
(SEQ ID NO: 29)
5′-CCCTTCGCTGACTCTC;
For:
(SEQ ID NO: 30)
5′-AATGGCCCCACCCCTATCT;
Rev:
(SEQ ID NO: 31)
5′-GCGGGCTGAGTCCGAAA.

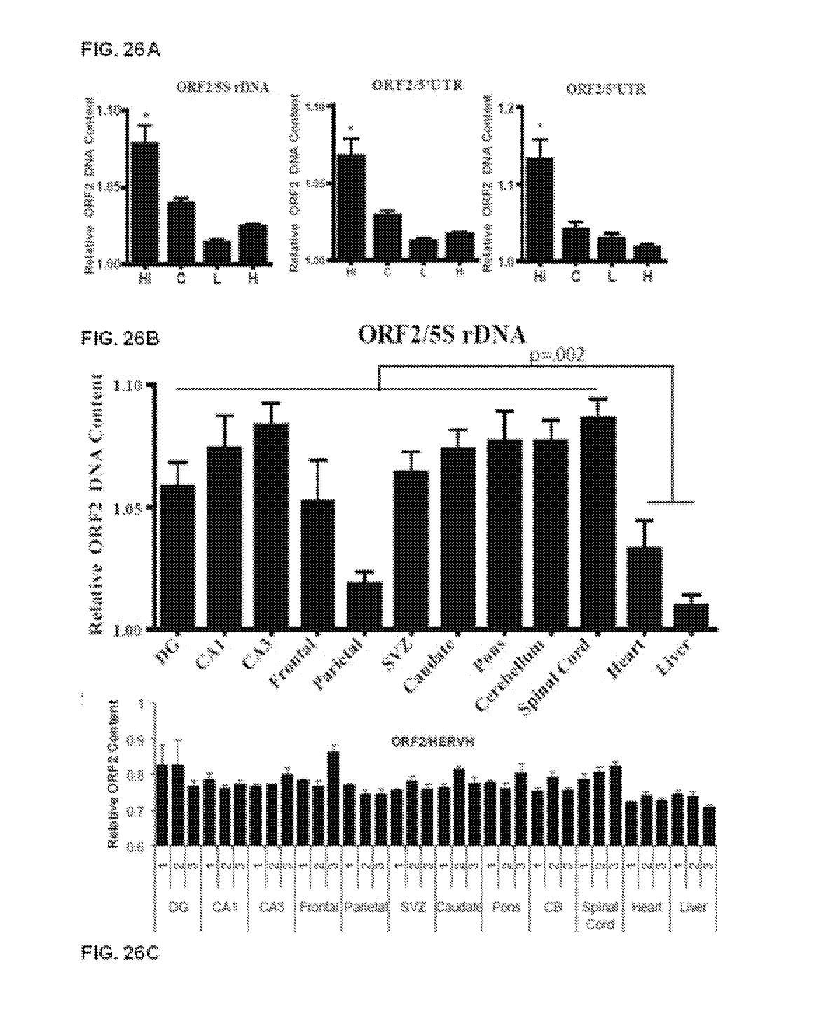

For each tissue type—hippocampus, cerebellum, heart, and liver—three individual tissue samples were taken and considered as repeated measures from each of the three individuals. The four tissues were compared utilizing a repeated measures one-way ANOVA with a Bonferroni correction. Statistically significant findings (p<0.05) are indicated in FIG. 17 and FIG. 26 by an asterisk. For three additional individuals, 10 different brain regions (CA1, CA3, dentate gyrus, frontal and partial cortex, caudate, subventricular zone, pons, cerebellum, and spinal cord) were sampled in addition to liver and heart. An unpaired two-tailed t-test was performed comparing the 10 brain regions and two somatic tissues, using an average of the three independent samples for each region (degrees of freedom=34). P values are indicated in figure legends (ORF2/5S rDNA p≦0.0001, ORF2/HERVH p≦0.002).

### Single Cell Assay to Quantify De Novo Line 1 Retrotransposition Events

### Nuclei Isolation

The protocol of Frisen (Spalding, et al. (2005) Cell 122(1): 133) has been optimized for nuclei isolation from very small amounts of tissue (<0.1 g) from fresh-frozen tissue samples stored at −80 C. Nuclei isolation is performed quickly as tissue is beginning to thaw. All solutions' are stored at 4° C. and the procedure is performed on ice. Frozen tissue (<0.5 g) is placed in 0.5 mL Lysis Buffer [0.32 M sucrose, 5 mM CaCl₂, 3 mM Mg acetate, 0.1 M EDTA, 10 mM Tris pH 8.0, 0.1% triton, 1 mM DTT], triturated slightly, and transferred to a small dounce homogenizer. Homogenization is accomplished in 10-12 strokes, after which the homogenate is transferred to 2 mL of Sucrose Buffer [1.8 M sucrose, 3 mM Mg acetate, Tris pH 8.0, 1 mM DTT]. The homogenizer is then rinsed with an additional 0.5 mL of Lysis buffer which is also added to the Sucrose Buffer containing homogenate. The mixture is then combined well by several inversions. Nuclei are separated from other tissue debris by centrifugation on a sucrose cushion. The 3 mL homogenate mixture from above is layered onto a cushion of 6 mL sucrose solution in a conical ultracentrifuge tube (Beckman part #358126). It is critical that layering be performed slowly and carefully, furthermore care is taken when loading these tubes into an ultracentrifuge rotor (Beckman SW28) so that a sharp interface between the cushion and the homogenate is maintained. Centrifugation at 12:9 K rpm, 4° C., for 2 hrs leads to a nuclei pellet in the centrifuge tube and cellular debris at the sucrose interface. After removing the sucrose cushion and cellular debris, nuclei are resuspended on 0.5 mL Nuclei Storage Buffer [15% sucrose, 2 mM MgCl₂, 70 mM KCl, 10 mM Tris pH 8.0, 1 mM DTT, 1× protease inhibitor cocktail with EDTA (Roche)]. Nuclei in this buffer are stored at 4 C until sorting, typically <4 days.

### Fluorescence Activated Cell Sorting (FACS)

Staining for nuclear antigens (e.g. NeuN) can be performed at this point following standard protocols. Otherwise, or afterward, stored nuclei are resuspended and diluted 1:5 in PBS containing 10 mM propidium iodide, then filtered through 20 um nylon mesh. Sorting is performed using a FACS Vantage SE DiVa (Becton-Dickenson). Gates are adjusted to obtain G1 nuclei with a diploid DNA content. Further dilution of the nuclei prep using PBS is performed if the solution is to concentrated. Sorting of highly concentrated preps leads to occasional sorting of debris rather than nuclei into wells. Nuclei are sorted into 96 well plates that are suitable for future analysis using quantitative PCR. Aside from actual sorting, care is taken to keep these plates clean by performing subsequent steps in a laminar flow hood. Before sorting, 5 μL of TE buffer (10 mM Tris pH 8.0, 1 mM EDTA) is placed in each well using a high throughput reagent dispenser (Multidrop 384, Thermo Scientific). The last column (8 wells) of the plate does not typically contain sorted nuclei and these wells are analyzed as DNA-free controls. Twelve plates are typically sorted for each tissue.

Quantitative PCR (qPCR)

Multiplex qPCR is performed using Taqman methodology (Applied Biosystems). Primer probe combinations are listed in an additional table. A master mix is prepared so that when 10 μL of mastermix is added to 5 μL containing a single nuclei in TE the final concentration of reagents is: 1× gene expression mastermix (Applied Biosystems), 1 mM control primers (e.g. 5S RNA), 0.1 mM experimental primers (e.g. L1 Orf2), and 0.2 mM taqman probes. The fluorophore 6FAM is typically used for the less abundant template (e.g. 5S RNA) and the fluorophore VIC is typically used for the more abundant template (e.g. Orf2). Mastermix is prepared and dispensed using the high throughput reagent dispenser in a laminar flow hood. The high throughput reagent dispenser requires an additional “dead” volume of 5 mL mastermix, this dead volume seems stable for several days and is often combined with “fresh” mastermix on subsequent days to reduce expenses.

The following primer pairs were used for quantitative PCR assays as well as the primer pairs previously listed in the methods section:

mouse:
ORF2-F:
(SEQ ID NO: 44)
5′-cctccattgttggtgggatt-3′;
Orf2-R:
(SEQ ID NO: 45)
5′-aaccgccagactgatttcca-3′;
ORF2 probe:
(SEQ ID NO: 4)
5′-cctgcaatcccaccaacaat-3′;
5′UTR-F:
(SEQ ID NO: 46)
5′-gagcactgaaactcagaggagaga-3′;
5′UTR-R:
(SEQ ID NO: 47)
5′ctggtgattctgttaccgtctatca-3′
5′UTR probe:
(SEQ ID NO: 48)
5′-ctgtctcccaggtctg-3′;
5S-F:
(SEQ ID NO: 49)
5′-ctcgtctgatctcggaagctaag-3′;
5S-R:
(SEQ ID NO: 50)
5′-gcggtctcccatccaagtac-3′;
5S probe:
(SEQ ID NO: 51)
5′-agggtcgggcctgg-3′;
additional 5′UTR primer pairs:
primer set A:
5′UTR-F:
(SEQ ID NO: 5)
5′-taagagagcttgccagcagaga-3′,
5′UTR-R:
(SEQ ID NO: 6)
5′-gcagacctgggagacagattct-3′;
primer set B:
5′UTR-F:
(SEQ ID NO: 7)
5′-agagagcttgccagcagagagt-3′,
5′UTR-R:
(SEQ ID NO: 8)
5′-gcagacctgggagacagattct-3′;
primer set C:
5′UTR-F:
(SEQ ID NO: 9)
5′-tgtctcccaggtctgctgataga-3′,
5′UTR-R:
(SEQ ID NO: 10)
5′-gattgttcttctggtgattctgttacc-3′;
Human:
ORF2-1F:
(SEQ ID NO: 52)
5′-tgcggagaaataggaacactttt-3′,
ORF2-1R:
(SEQ ID NO: 53)
5′-tgaggaatcgccacactgact-3′;
ORF2-1Probe:
(SEQ ID NO: 11)
5′-ctgtaaactagttcaaccatt-3′;
ORF2-2F:
(SEQ ID NO: 54)
5′-caaacaccgcatattctcactca-3′;
ORF2-2R:
(SEQ ID NO: 55)
5′-cttcctgtgtccatgtgatctca-3′;
ORF2-2 probe:
(SEQ ID NO: 56)
5′-aggtgggaattgaac-3′;
L15′UTR-1F:
(SEQ ID NO: 57)
5′-gaatgattttgacgagctgagagaa-3′;
L15′UTR-1R:
(SEQ ID NO: 58)
5′-gtcctcccgtagctcagagtaatt-3′;
L15′UTR-1 probe:
(SEQ ID NO: 59)
5′-aaggcttcagacgatc-3′;
L15′UTR-2F:
(SEQ ID NO: 60)
5′-acagctttgaagagagcagtggtt-3′;
L15′UTR-2R:
(SEQ ID NO: 61)
5′-agtctgcccgttctcagatct-3′;
L15′UTR-2 probe:
(SEQ ID NO: 62)
5′-tcccagcacgcagc-3′;
SATA-F:
(SEQ ID NO: 63)
5′-ggtcaatggcagaaaaggaaat-3′;
SATA-R:
(SEQ ID NO: 64)
5′-cgcagtttgtgggaatgattc-3′;
(SEQ ID NO: 65)
5′-tcttcgtttcaaaactag-3′;
HERVH-F:
(SEQ ID NO: 66)
5′-aatggccccacccctatct-3′;
HERH-R:
(SEQ ID NO: 67)
5′-gcgggctgagtccgaaa-3′;
HERVH-probe:
(SEQ ID NO: 68)
5′-cccttcgctgactctc-3′.

### Data Analysis

qPCR results are obtained as a “Ct” value. This is the cycle number at which amplification of each template crosses a defined threshold. The threshold is defined as a point during which exponential amplification is observed, typically at 0.1 arbitrary fluorescence units. A dCt value is obtained for each well by subtracting the Ct of the more abundant template from the Ct of the less abundant template (e.g. dCt=Ct_{5S}−Ct_{ORF2}). In order to calculate the number of de novo RT events one must calculate a fold-change in dCt in an experimental (e.g. brain, iPS-derived neurons) versus a control (e.g. heart, small intestine, pre-iPS fibroblasts). The first component of calculating fold-change is a “ddCt,” obtained by subtracting the dCt from one control nucleus from one experimental nucleus. These may be paired individually either randomly or by rank; or an average of control nuclei can be used for each experimental nucleus. Fold-change is calculated as 2̂−ddCt; 2 assume perfect efficiency in the reactions, if the reactions are found to be less efficient the value 2 should be changed accordingly (e.g. 95% efficiency=1.9̂−ddCt). The number of initial L1 sequences in the reference genome is obtained using BLAT (UC Santa Cruz), and de novo events are calculated based on a fold-change from this reference value.

### Results

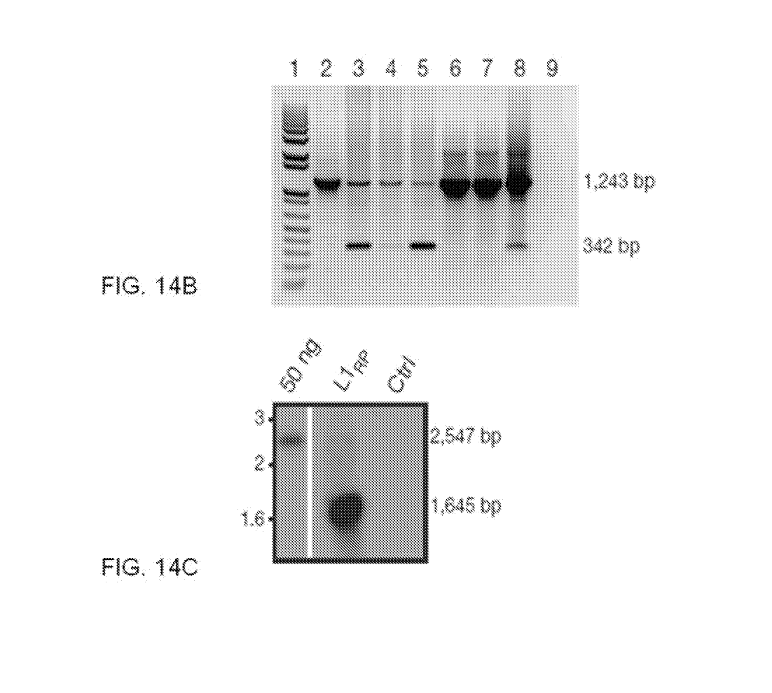

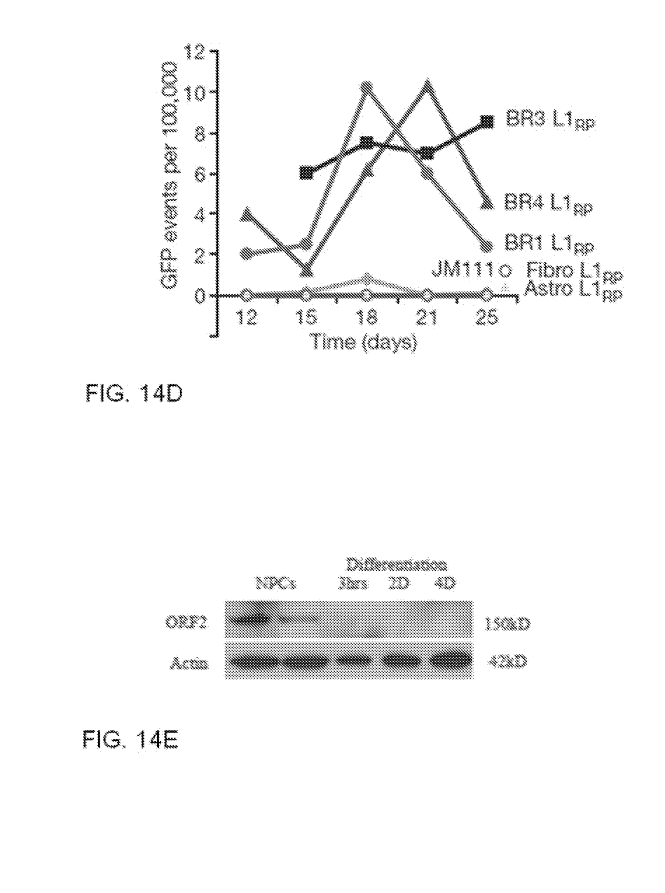

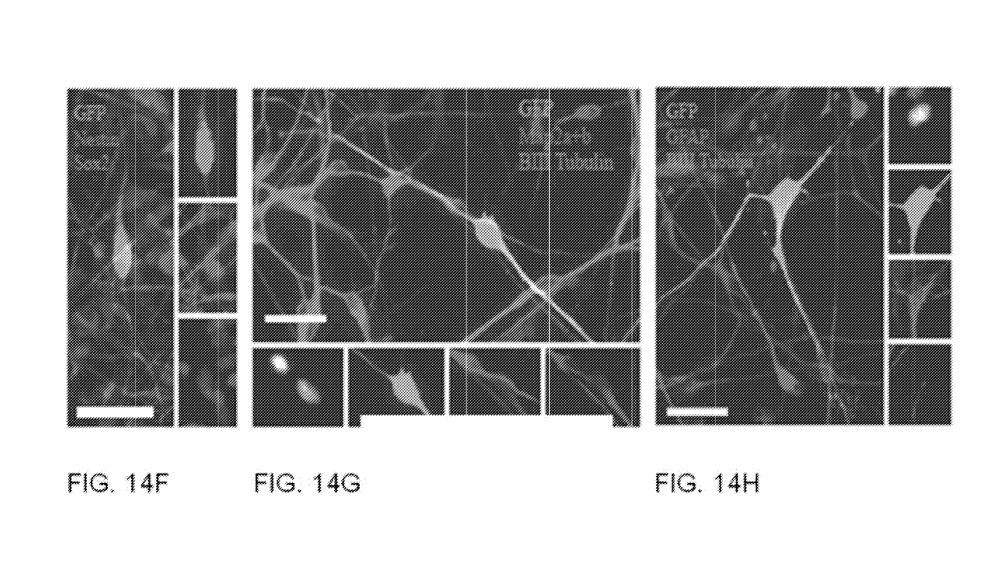

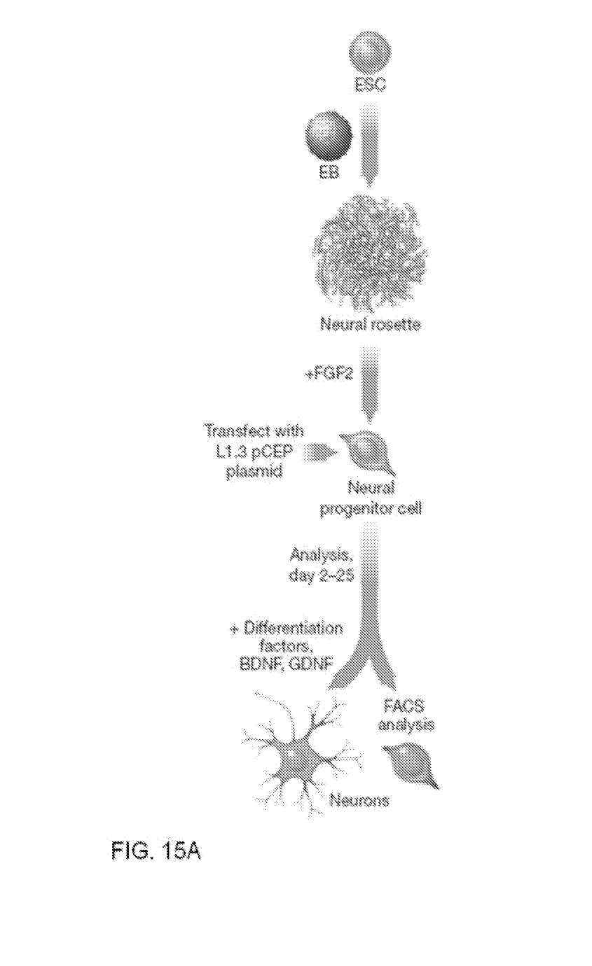

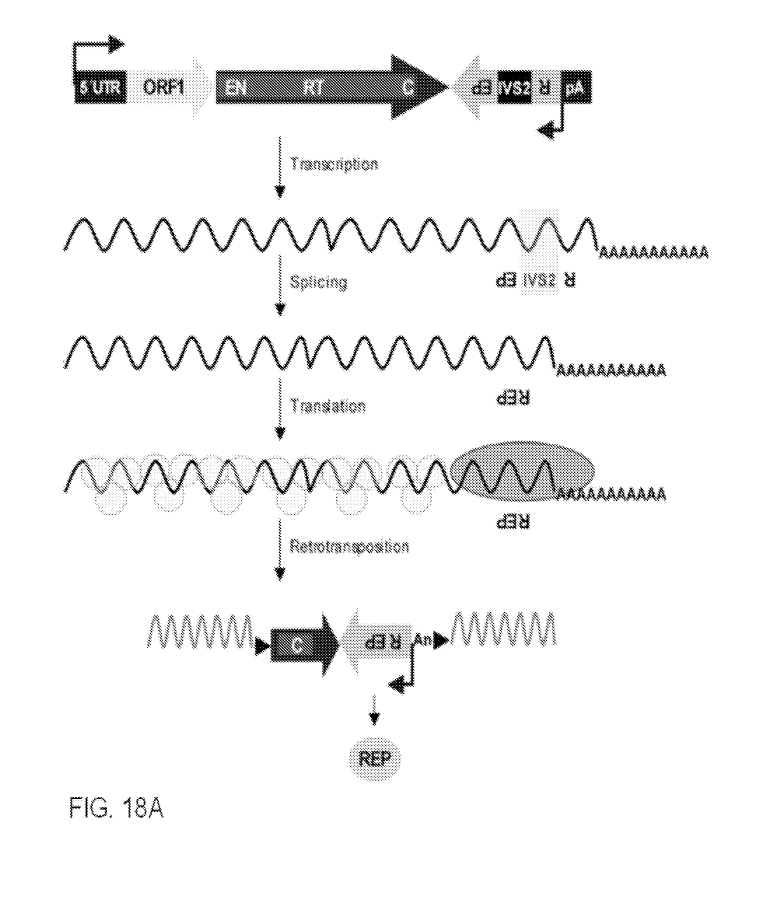

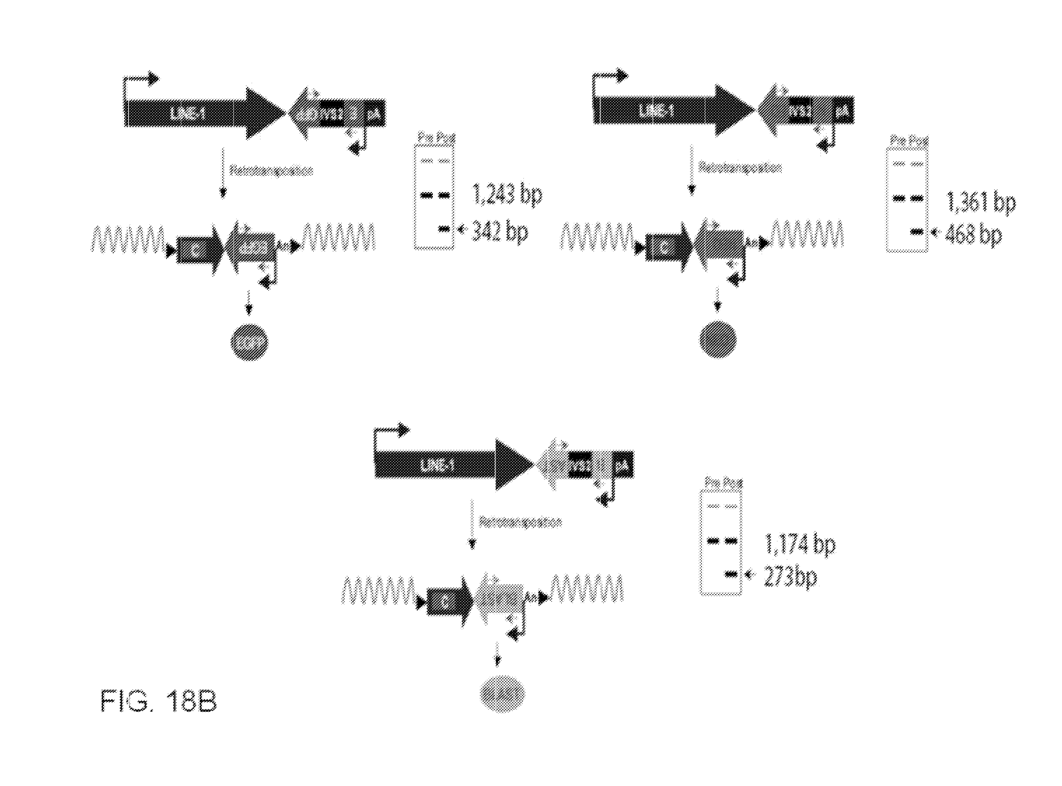

The human nervous system is complex, containing approximately 10¹⁵ synapses with a vast diversity of neuronal cell types and connections that are influenced by complex and incompletely understood environmental and genetic factors (35). Neural progenitor cells (NPCs) give rise to the three main lineages of the nervous system: neurons, astrocytes, and oligodendrocytes. To determine if human NPCs can support L1 retrotransposition, human fetal brain stem cells (hCNS-SCns) (FIG. 14A) (36) were transfected with an expression construct containing a retrotransposition-competent human L1 driven from its native promoter (RC-L1; L1_{RP}). The RC-L1 also contains a retrotransposition indicator cassette in its 3′ UTR, consisting of a reversed copy of the enhanced green fluorescent protein (EGFP) expression cassette, which is interrupted by an intron in the same transcriptional orientation as the RC-L1 (37-40). The orientation of the cassette ensures that EGFP-positive cells will arise only if the RC-L1 undergoes retrotransposition (FIG. 18A).

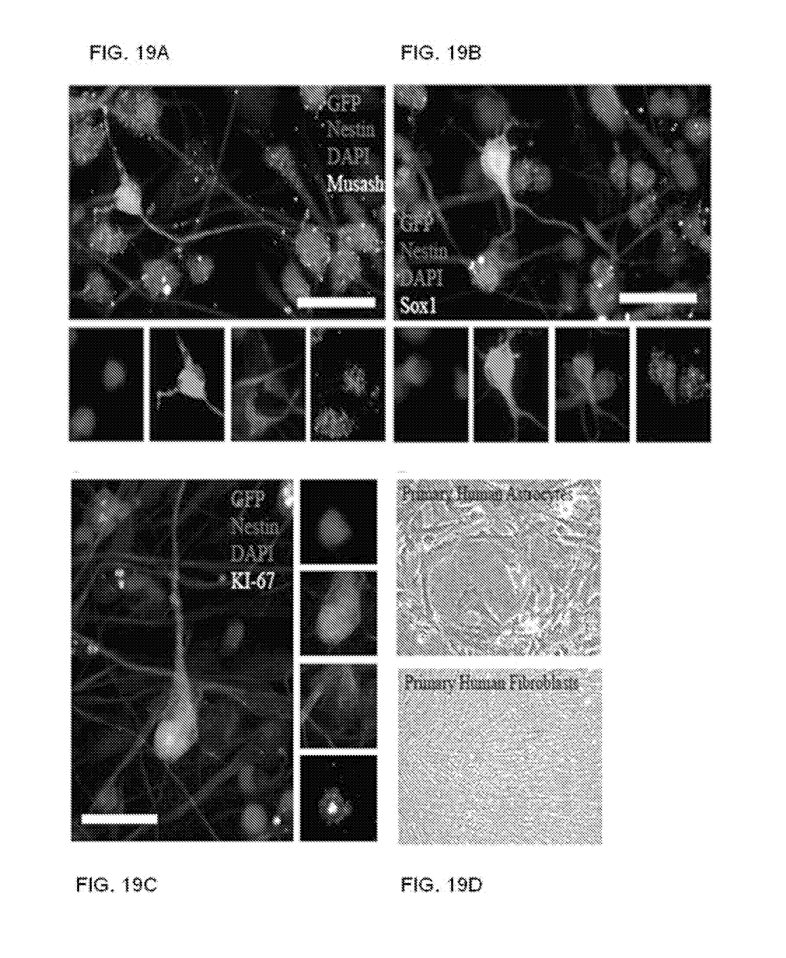

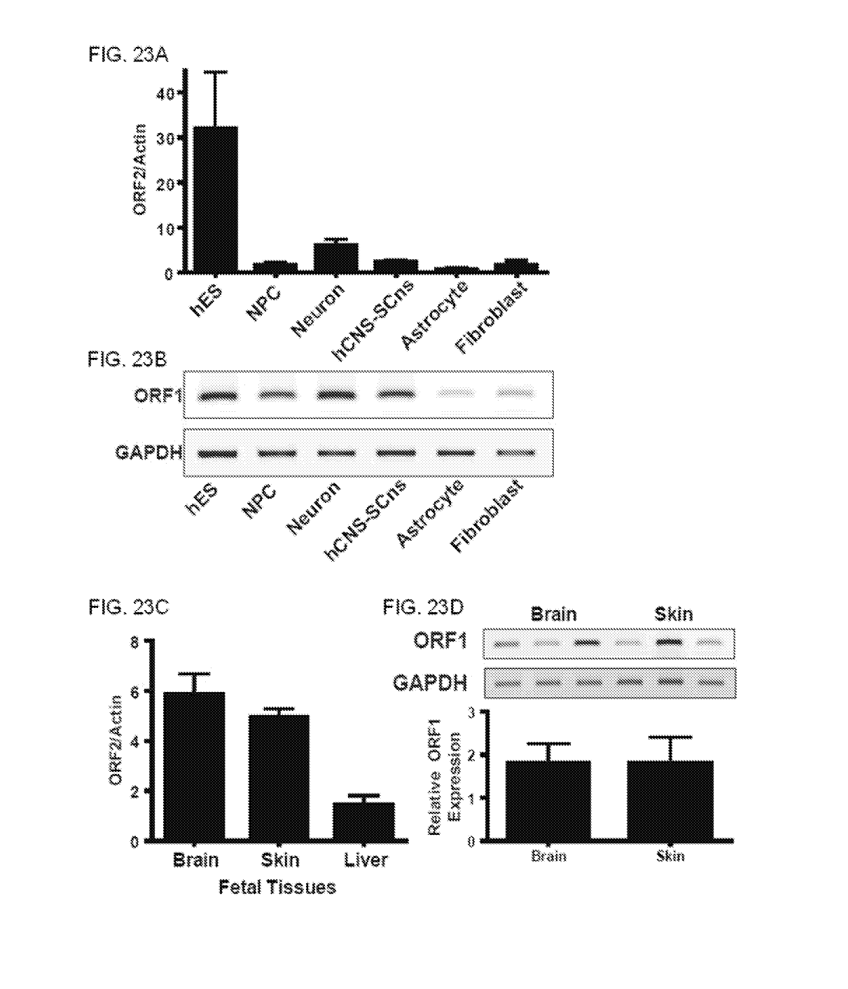

A low level of L1_{RP} retrotransposition, averaging 8-12 events per 100,000 cells, was observed in three different hCNS-SCns lines (BR1, BR3 and BR4; FIG. 14D). By comparison, an L1 containing two missense mutations in the ORF1-encoded protein (JM111/L1_{RP}) (38,41) did not retrotranspose (FIG. 14B,D). Controls demonstrated precise splicing of the intron from the retrotransposed EGFP gene (FIGS. 14B,18,21) and indicated that L1 retrotransposition events were detectable by both PCR and Southern blotting 3 months post-transfection (FIG. 14C). Moreover, RT-PCR revealed that hCNS-SCns express endogenous L1 transcripts and that some transcripts are derived from the human-specific (L1Hs) subfamily (37,42,43) (FIG. 23A-B; Table 5; Table 6). To determine if L1 retrotransposition occurred in undifferentiated cells, immunocytochemical localization of cell type restricted markers in EGFP-positive hCNS-SCns were conducted. These cells expressed neural stem cell markers, including Sox2, Nestin, Musashi-1 and Sox1 (FIGS. 14E, 19A-B), and some co-labeled with Ki-67, indicating that they continued to proliferate (FIG. 19C). EGFP-positive hCNS-SCns also could be differentiated to cells of both the neuronal and glial lineages (FIG. 14F,G). Notably, L1_{RP} did not retrotranspose using the same experimental conditions in primary human astrocytes or fibroblasts, although a low level of endogenous L1 expression was detected in both cell types (FIGS. 14D, 19D-E, 23A-B).

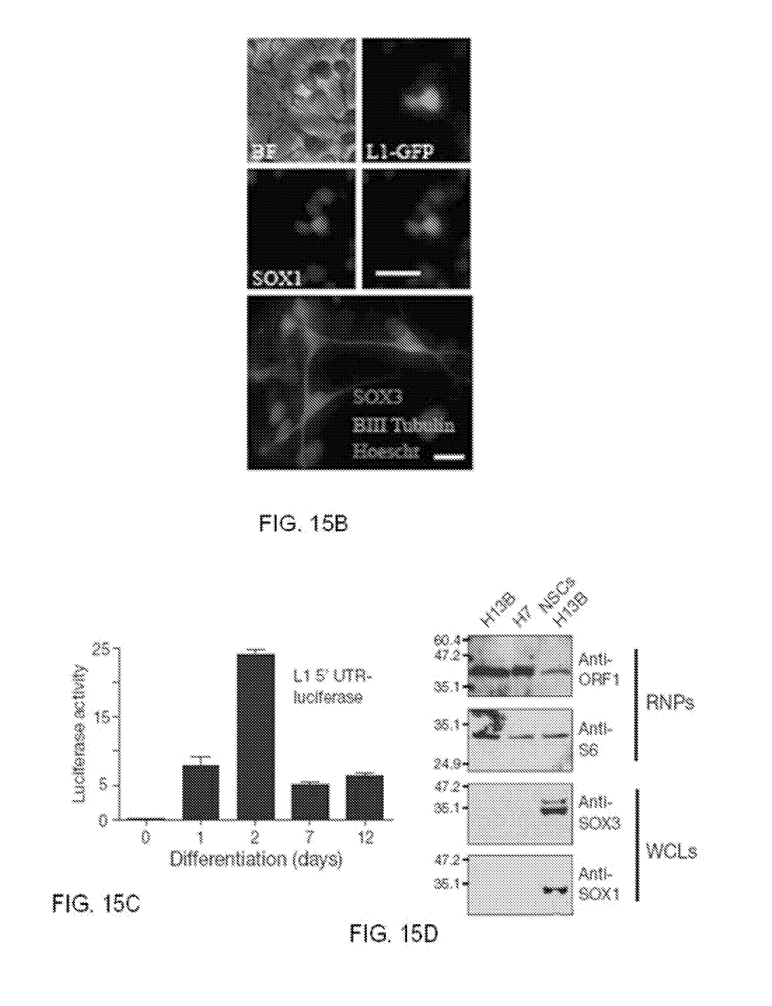

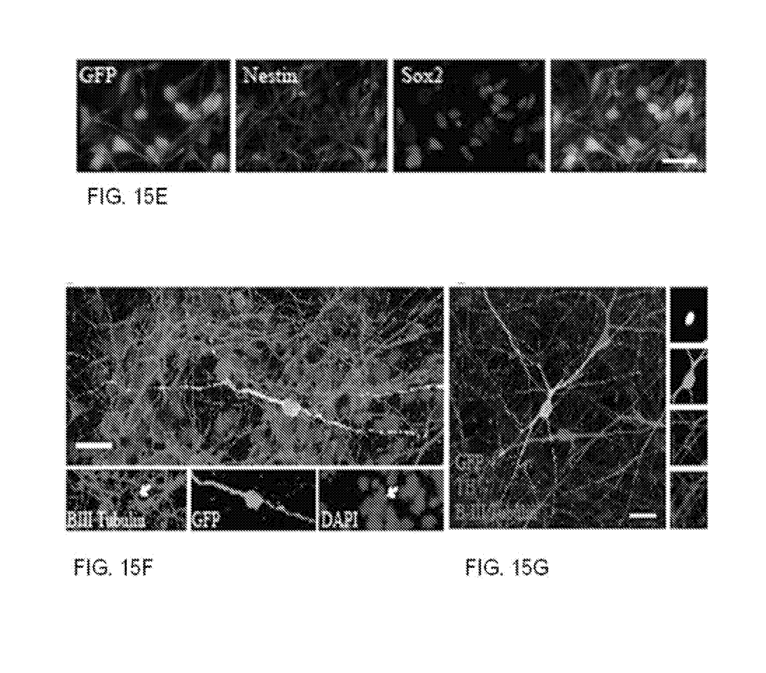

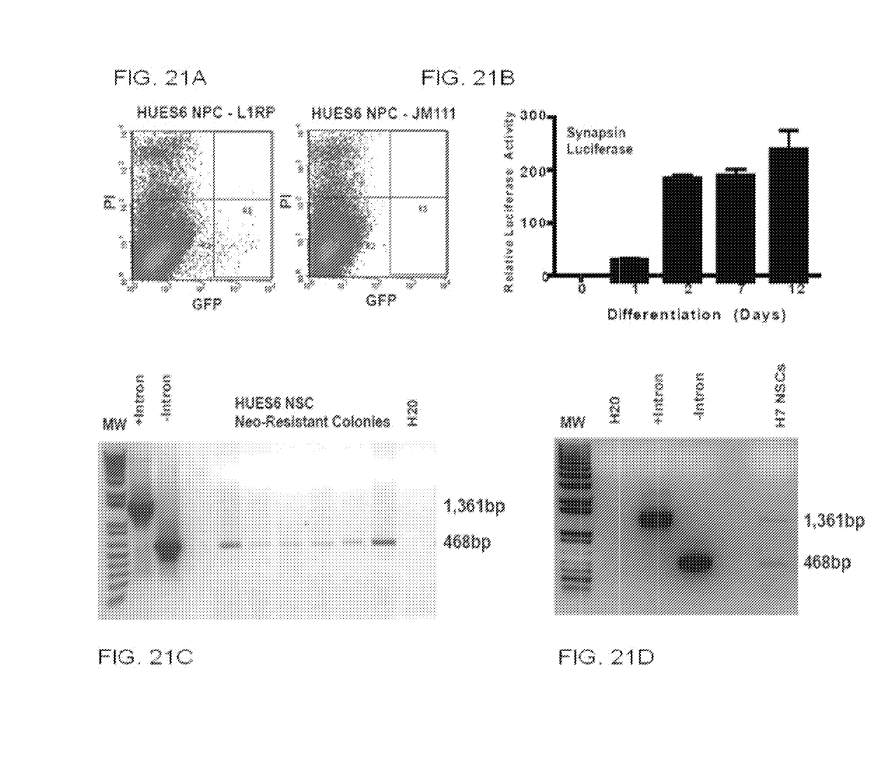

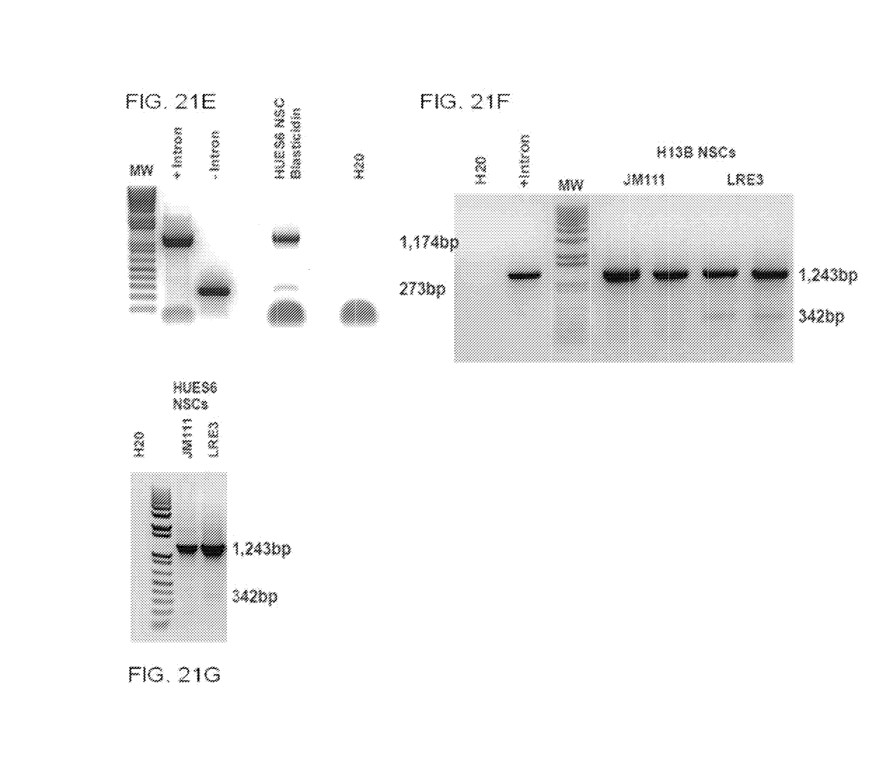

Next two different protocols were used to derive NPCs from five human embryonic stem cell lines (hESCs; FIG. 15A). As in previous study (34), NPC differentiation led to a ˜25-fold increase in L1 promoter activity over a 2-day period and then declined (FIG. 15C); there also was a ˜250-fold increase in synapsin promoter activity during differentiation (FIG. 21B). H13B-derived NPCs expressed both endogenous L1 RNA and ORF1p, although the level of ORF1p expression was less than in the H13B hESC line (FIG. 15D). HUES6-derived NPCs also expressed endogenous L1 RNA (FIG. 23A-B) and sequencing indicated some transcripts are derived from the L1Hs subfamily (Table 5; Table 6). Similar studies performed with fetal brain, liver, and skin samples showed evidence of endogenous L1 transcription (FIG. 23C-D; Table 5; Table 6). RC-L1 retrotransposition was readily detected at varying efficiencies in hESC-derived NPC lines (Table 2; lab G and M; FIGS. 18, 21F-G). Again, it was determined that JM111/L1_{RP} could not retrotranspose (Table 2), that EGFP-positive NPCs expressed canonical neural stem cell markers (FIGS. 15B, 15E, 20C, 20D), and that EGFP-positive HUES6-derived NPCs could be differentiated to cells of both the neuronal and glial lineages (FIGS. 15F, 20E-F). The variability in retrotransposition efficiencies in hES-derived NPCs likely depended on multiple factors (see Table 2, for specific details).

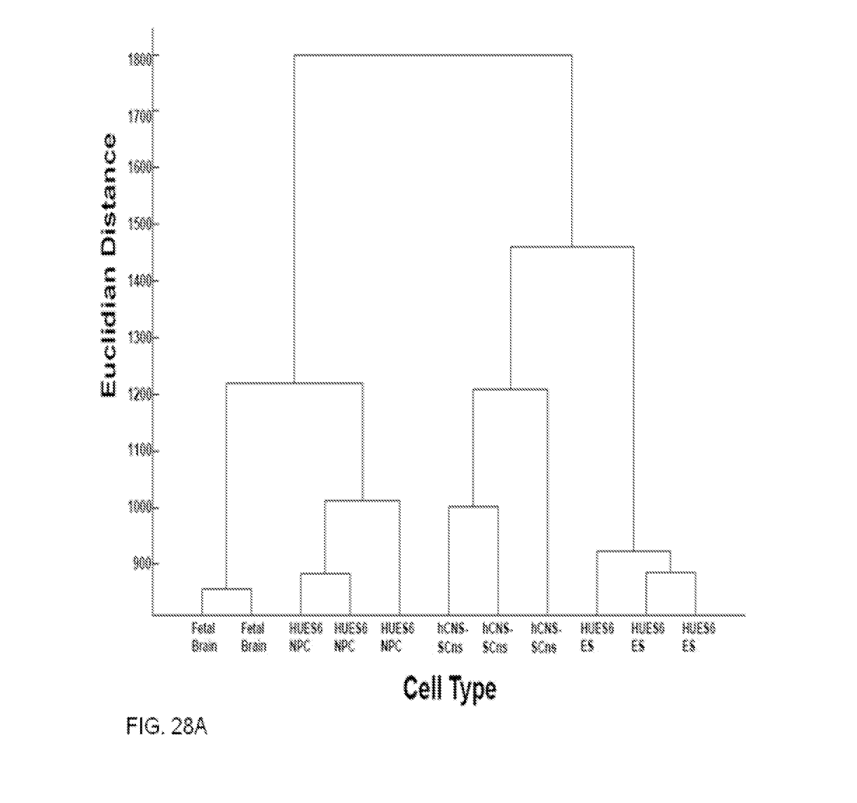

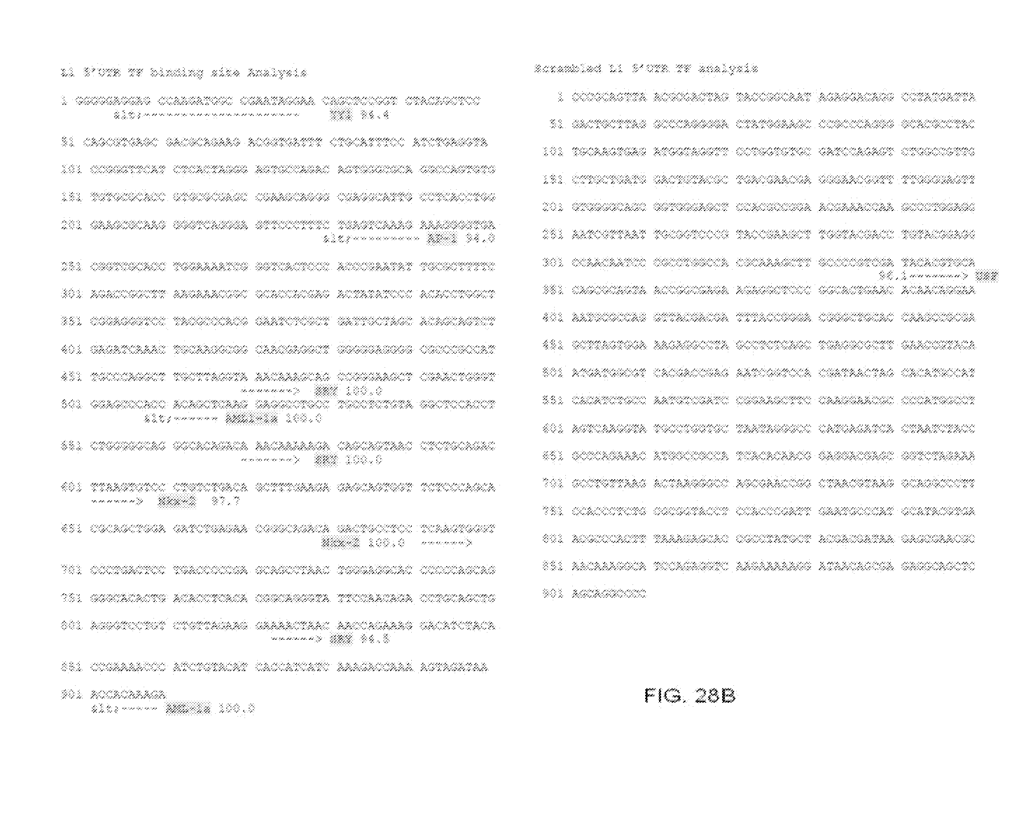

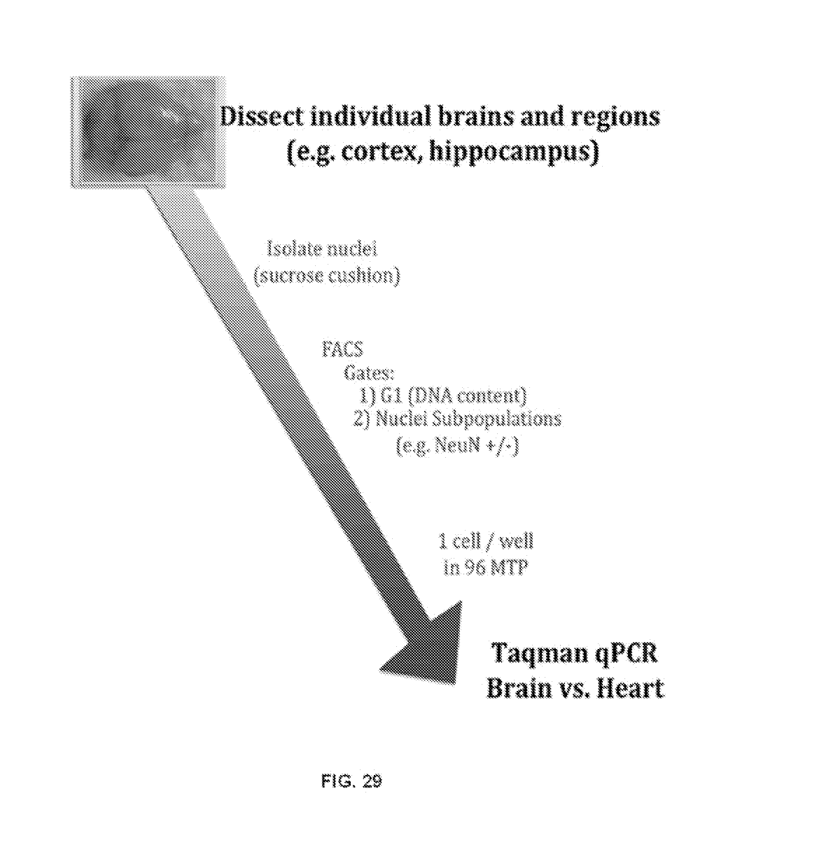

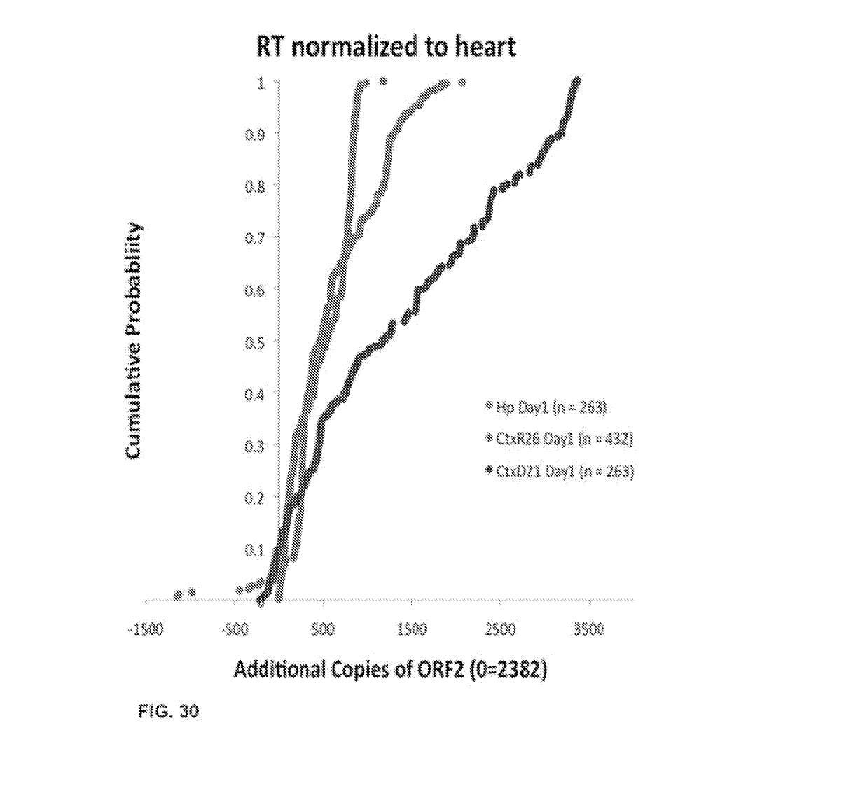

Characterization of EGFP-positive neurons revealed that some expressed subtype-specific markers (tyrosine hydroxylase (FIG. 15G) and GABA (data not shown)) and whole-cell perforated patch clamp recording demonstrated that some HUES6-derived NPCs are functional (FIG. 2H-K; n=4 cells). Finally, it was demonstrated that an RC-L1 tagged with neomycin or blasticidin retrotransposition indicator cassettes could retrotranspose in NPCs (FIGS. 18,21C-E) (38,44). Some G418-resistant foci also expressed SOX3 and could be differentiated to a neuronal lineage (FIG. 15B). Next 19 retrotransposition events from EGFP-positive NPCs were characterized (FIG. 24B; Table 3). Comparison of the pre- and post-integration sites demonstrated that retrotransposition occurred into an actual or inferred L1 endonuclease consensus cleavage site (5′-TTTT/A and derivatives). Five events were flanked by target site duplications, and no large deletions were detected at the insertion site (38,42,45) (FIG. 24B; Table 3). Interestingly, 16 of 19 retrotransposition events were fewer than 100 kB from a gene and some occurred in the vicinity of a neuronally expressed gene (34,45,46). Notably, consistently higher L1 retrotransposition efficiencies were observed in hESC-derived NPCs when compared to fetal NPCs. A Euclidian distance map based on exon-array expression analysis (47) indicated that hCNS-SCns cluster closer to HUES6 cells, whereas HUES6-derived NPCs cluster closer to fetal brain (FIG. 28A). Thus, hESC-derived NPCs and hCNS-SCns may represent different developmental stages in progenitor differentiation. That being stated, it is concluded that engineered human L1s can retrotranspose in human NPCs.

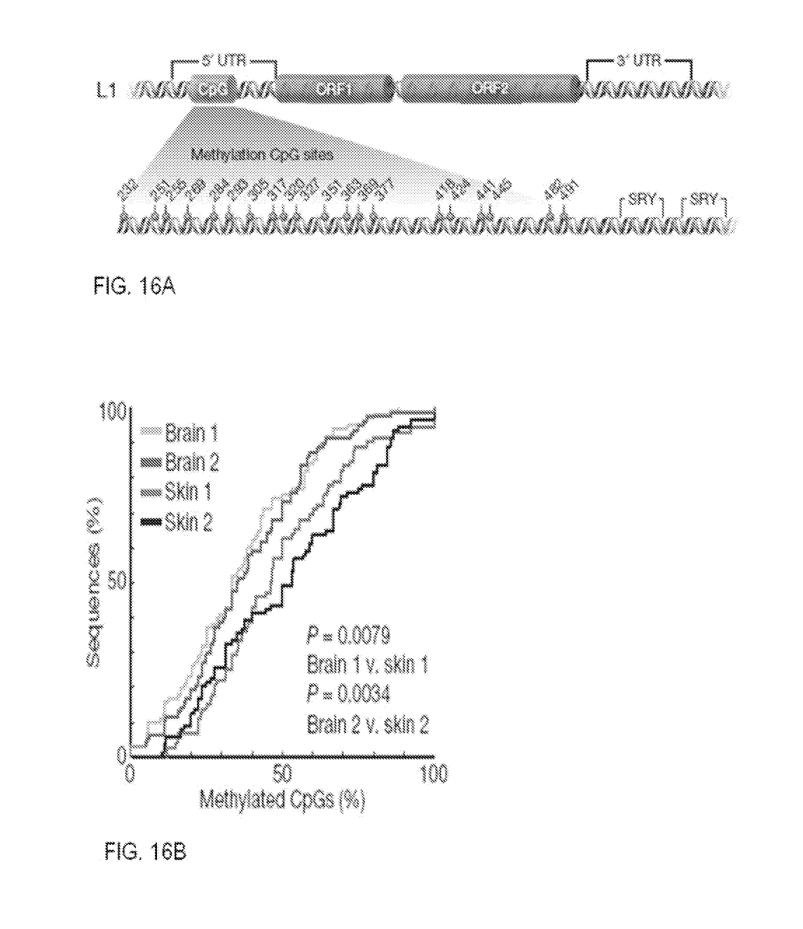

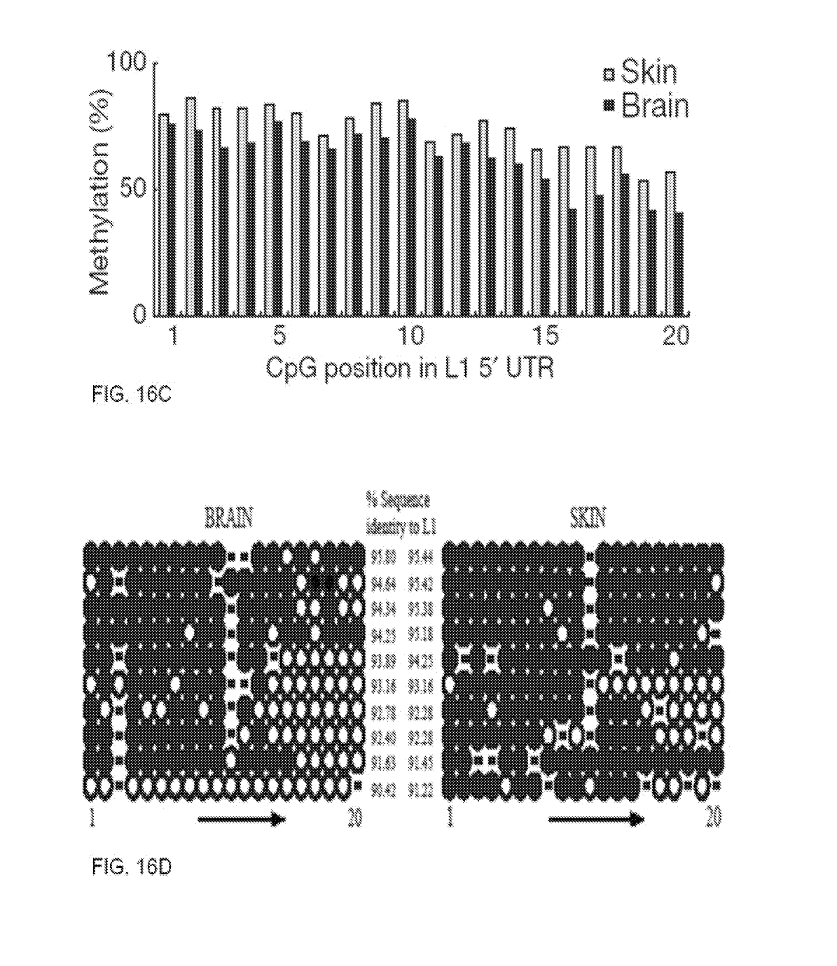

Several studies have reported an inverse correlation between L1 expression and the methylation status of the CpG island in their 5′ UTRs (48,49). Thus, bisulfite conversion analyses on genomic DNAs derived from matched brain and skin tissue samples from two 80- to 82-day-old fetuses were performed (FIG. 16A; one male/one female sample). A portion of the L1 5′UTR containing 20 CpG sites was then amplified and the resultant amplicons were sequenced. Interestingly, the L1 5′ UTR exhibited significantly less methylation in both brain samples when compared to the matched skin sample (Two-sample Kolmogorov-Smirnov test P≦0.0079 day 80 female, P≦0.0034 day 82 male; FIG. 16B). The analysis of individual L1 5′ UTR sequences, demonstrated the greatest variation between the brain and skin at CpG residues located near the 3′ end of the amplicon, and six amplicons from the brain samples were unmethylated (FIGS. 16E,25A-B). Restricting this analysis to 10 L1s from both brain and skin with highest sequence homology to an RC-L1 revealed 19/20 sequences were derived from the L1Hs subfamily (data not shown), and one L1Hs element from the brain was completely unmethylated (FIG. 16C). In all cases, control experiments showed that the bisulfite conversion efficiency was >90% (FIG. 25C).

Previous data suggested that Sox2 and MeCP2 could associate with the L1 promoter and repress L1 transcription under some experimental conditions (34,50). Two putative SRY/Sox2 binding sites are located in the L1 5′ UTR immediately 3′ to the CpG island (FIGS. 16A, 28B) (51). Thus, chromatin immunoprecipitation (ChIP) for Sox2 and MeCP2 were performed in hCNS-SCns, HUES6-derived NPCs, and HUES6-derived neurons. Sox2 associated with the L1 5′ UTR in a pattern that correlates with the decrease in SOX2 expression observed during neural differentiation (FIGS. 16D,21H). MeCP2 expression was lower in both hCNS-SCns and HUES6-derived NPCs than in neurons (FIG. 21H), and both hCNS-SCns and HUES6-derived NPCs expressed similar levels and types of L1 transcripts (FIGS. 23A-B). However, higher levels of MeCP2 were detected in association with the L1 promoter in hCNS-SCns than in HUES6-derived NPCs. It may be that less L1 promoter methylation in the developing brain may correlate with increased L1 transcription and perhaps L1 retrotransposition, and the differential interaction of Sox2 and MeCP2 with L1 regulatory sequences may modulate L1 activity in different neuronal cell types.

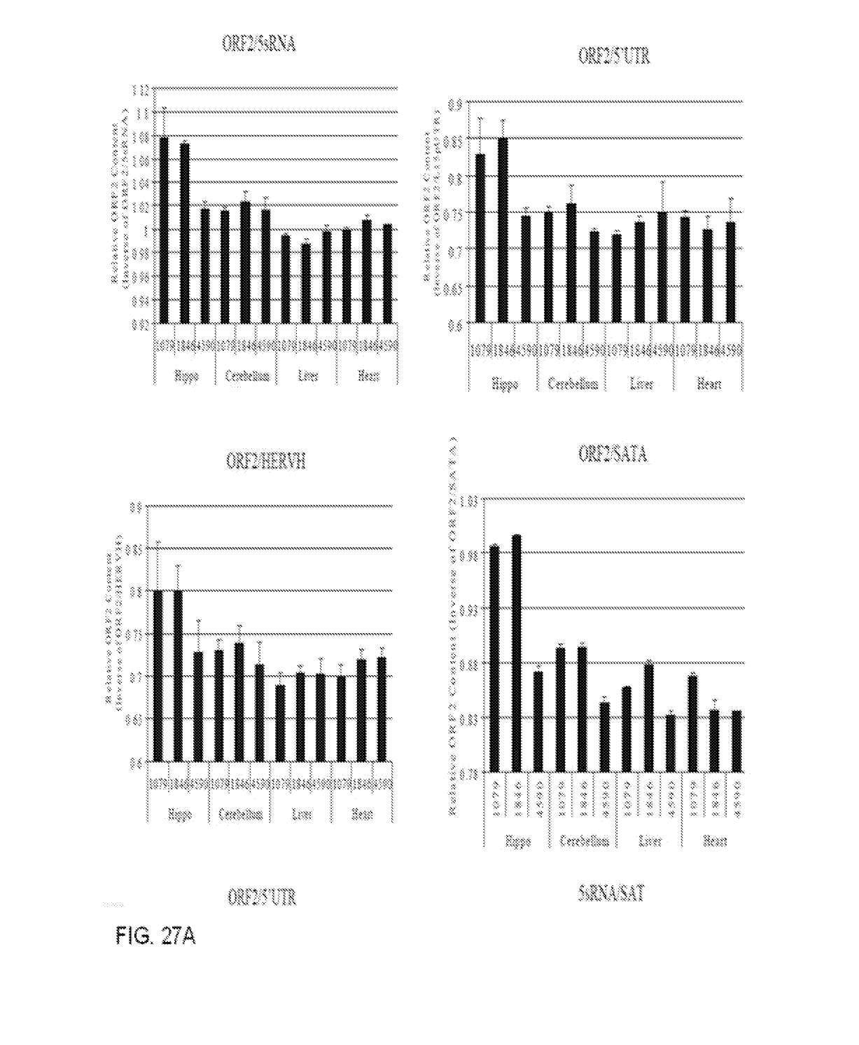

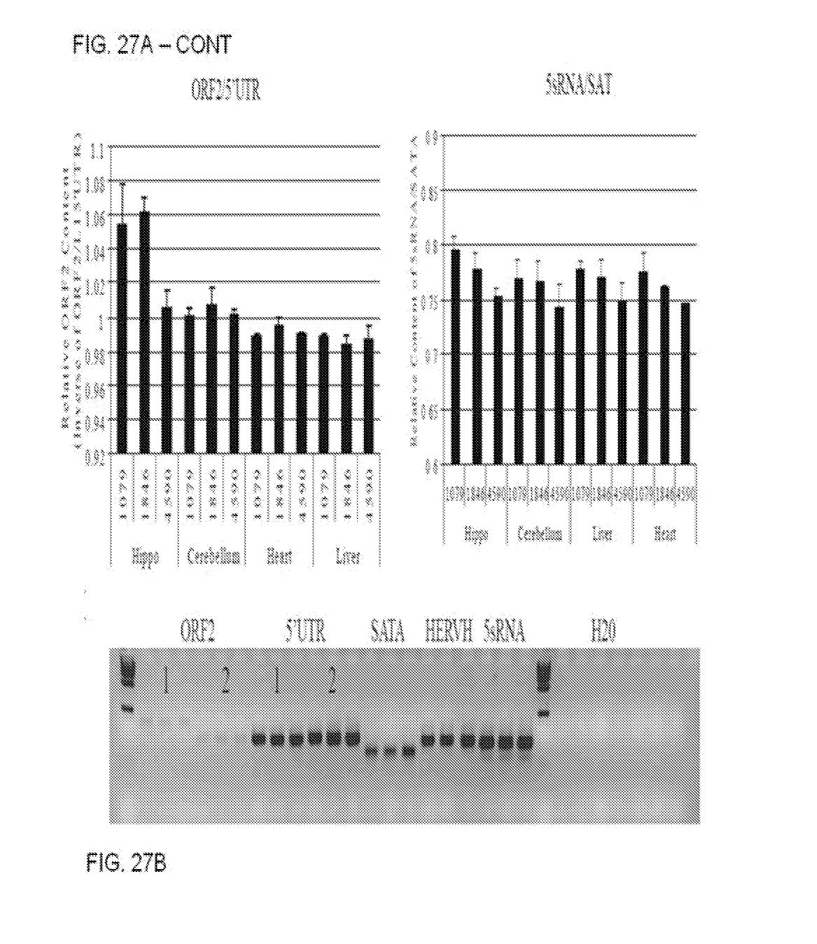

Although NPCs are useful to monitor L1 activity, they only allow monitoring a single L1 expressed from a privileged context. By comparison, the average human genome contains ˜80-100 active L1s whose expression may be affected by chromatin structure (37). Therefore, a quantitative multiplexing PCR strategy was developed to investigate endogenous L1 activity in the human brain, hypothesizing that active retrotransposition would result in increased L1 content in the brain as compared to other tissues (FIG. 17A). Briefly, Taqman probes against a conserved 3′ region of ORF2 were designed (conjugated with the VIC fluorophore), in addition to a number of control probes (conjugated with the 6FAM fluorophore). Controls were designed against the L1 5′UTR and other non-mobile DNA sequences in the genome that are higher (e.g., a satellite (52)) or lower in copy numbers (e.g., HERVH and 5S rDNA gene) than ORF2. In addition since the majority of L1 retrotransposition events are 5′ truncated (42,53,54), it was reasoned that the L1 5′ UTR probes should detect a smaller copy number increase than the L1 ORF2 probes. Each probe set amplified a single product of the predicted size (FIG. 27B). Moreover, sequencing PCR products derived from both ORF2 probe sets revealed enrichment for members of the L1Hs subfamily (Tables 4A,B). Next genomic DNA was isolated from the hippocampus, cerebellum, liver and heart from three adult humans. A statistically significant increase in L1 ORF2 content in the hippocampus was consistently observed when compared to heart and liver samples from the same individual (FIGS. 17B-C, 26A, 27A). Notably, two individuals (1079 & 1846) showed more dramatic copy number differences than a third (4590) (FIG. 27A). Controls demonstrated that the ratio of the 5S rDNA gene to a satellite DNA between each tissue remained relatively constant (FIG. 17E). This analysis was extended to 10 brain regions from three additional individuals (FIGS. 17D,26B). The samples were derived from the frontal and parietal cortex, spinal cord, caudate, CA1 and CA3 areas of the hippocampus, and pons, as well as from the hippocampal dentate gyrus (DG) and the subventricular zone (SVZ) (55). As above, there was marked variation between different brain areas and between individuals (FIG. 26C). However, an unpaired t-test comparing all the grouped brain samples to the heart and liver DNA again revealed a small, but statistically significant increase in ORF2 content in the brain (FIG. 17D).

To independently corroborate the observed increase in L1 copy number in the hippocampus and cerebellum samples, 80 pg of liver and heart genomic DNA were spiked (approximately 12 genomes) from individual 1846 with a calculated quantity of L1 plasmid, then the multiplexing approach was repeated to assay ORF2 quantity relative to 5S rDNA internal control (FIG. 17F). Three replications of this experiment indicated that the hippocampus samples contained approximately 1,000 more L1 copies than the heart or liver genomic DNAs, suggesting a theoretical increase in ORF2 of approximately 80 copies/cell. The spiked L1 copies were in the form of a plasmid, which likely affects the copy number estimates, providing an estimate of relative change and not precise quantification of the absolute number of L1s per cell. Ultimately, proof that endogenous L1s are retrotransposing in the brain requires identification of new retrotransposition events in individual somatic cells. The large degree of variability in L1-ORF2 copy numbers between brain regions and individuals may represent unsystematic rates of L1 retrotransposition or an additional level of regulation that requires further elucidation. That being stated, the in vitro findings in NPCs coupled with the observed L1-ORF2 copy number changes in the brain make it tempting to speculate that somatic retrotransposition events occur during early stages of human nervous system development. This study contributes to a body of evidence indicating that engineered L1s can retrotranspose during early development, and in selected somatic cells (34,39,56-58).

### Tables

Experimental results are summarized in the tables referred to herein.

Table 1 provides the variation of L1 ORF2 sequences in the human brain and heart tissue from normal and RTT patients; numbers correspond to inverse CT values and were obtained from the multiplex qPCR strategy.

Table 2 provides the results of L1 retrotransposition assays in hESC-derived NPCs. From left to right, column 1 indicates the hESCs cell line from which NPCs were derived, column 2 indicates the lab where the experiments were performed, column 3 indicates if selection (puromycin 0.2 μg/mL) was used in the assay, and column 4 indicates the percentage of EGFP-expressing cells with s.d. The variation likely depends on the individual NPC preparation, the differentiation protocol, whether the NPCs were subjected to puromycin selection prior to assaying for retrotransposition, and if the resultant retrotransposition event was subjected to silencing (indicated by the (* in column 1)). It was observed that L1 retrotransposition events could be efficiently silenced in some hESC-derived NPCs (column 1, marked H13B*). This silencing could be overcome by treating the cells with histone deacetylase inhibitors and it may reflect idiosyncrasies that arise during the differentiation protocol. In the table: a—Gage (G) or Moran (M) groups. HUES6 is a private cell line all others are federally approved; b—puromycin, 0.2 ugfmL; (*) these NPC derivations exhibited silencing of the Li insertions, whereas other NPCs derivations did not; (*) either a JM1 il/L1RP construct or untransfected samples (in triplicate) were used to determine baseline background fluorescence; UB=where the ubiquitin C promoter drives the expression of the LI. In all other experiments, the LRE3 is driven from its native 5′ UTR.

Table 3 provides an analysis of L1 insertions in hESC-derived NPCs. From left to right: column 1: if the insertion was characterized from a clone or from FACS-sorted cells (derivations 1 and 2 are from separate NPC derivations, and separate transfections of L1); column 2: if the insertion characterization was full or partial; column 3: the truncation site of the retrotransposed tagged L1; column 4: the estimated length of the poly (A) tail; column 5: the sequence of the actual or inferred LINE-1 endonuclease bottom strand cleavage site; column 6: the chromosomal locus of the insertion; column 7: the insertion target site of the tagged L1. Note that eight insertions were characterized completely; however, only the 3′ end was characterized of the remaining insertions because the restriction enzyme utilized in the ligation step of the inverse PCR protocol was also present in the retrotransposed L1 sequence. In the table: 8—clone, or from either the first or second independent NPC derivation; b—full insertions: both 5 and Y documented; partial insertions: only 3′ genomic location isolated; c—the nucleotide position in RC-L1mEGFPI where the retrotransposition event is truncated; NA=not analyzed.

Tables 4A and 4B provide a sequencing analysis of QPCR genomic DNA products. PCR products from both ORF2#1 and ORF2#2 primer sets were cloned and sequenced from PCR reactions run with both hippocampus and liver genomic DNA. Percentage sequence identity to an RC-L1 consensus sequence was determined. Sequence analysis using the UCSC genome browser and Repeatmasker indicates that the majority of amplified sequences belong to the L1Hs subfamily of elements. Notably, due to their short length, some amplicons could not be definitively assigned to a single L1 subfamily.

Tables 5A and 5B provide a sequencing analysis of QPCR products from L1 RT-PCR. Quantitative RT-PCR products from ORF2 #1 primer sets were cloned from three sample types: fetal brain, hCNS-SCns, and HUES6-derived NPCs. Percentage sequence identity to an RC-L1 consensus was determined, and sequence analysis using UCSC genome browser and Repeatmasker indicated that most sequences belonged to the L1Hs subfamily of elements. Complete sequence of the QPCR product is indicated in Table 5A.

Tables 6A and 6B provide a sequence analysis of actively transcribed ORF1 fragments from RT-PCR. RT-PCR fragments (see FIG. 23) were cloned and sequenced from three samples: fetal brain, hCNS-SCns, and HUES6-derived NPCs. Percentage sequence identity to an active RC-L1 (L1.3) was determined, as well as sequence analysis using the UCSC genome browser and Repeatmasker. In addition, since these are larger fragments than those resulting from QPCR, most mapped to a unique genomic location. Complete sequence of the RT-PCR product is indicated in Table 6A.

### REFERENCES

The following documents are incorporated by reference herein.
1 Lander, E. S. et al. Initial sequencing and analysis of the human genome. Nature 409, 860-921 (2001).
2 Gibbs, R. A. et al. Genome sequence of the Brown Norway rat yields insights into mammalian evolution. Nature 428, 493-521 (2004).
3 Waterston, R. H. et al. Initial sequencing and comparative analysis of the mouse genome. Nature 420, 520-562 (2002).
4 Lutz, S. M. et al. Allelic heterogeneity in LINE-1 retrotransposition activity. Am J Hum Genet 73, 1431-1437 (2003).
5 Seleme Mdel, C. et al. Extensive individual variation in L1 retrotransposition capability contributes to human genetic diversity. Proc Natl Acad Sci USA 103, 6611-6616 (2006).
6 Grimaldi, G., Skowronski, J. & Singer, M. F. Defining the beginning and end of KpnI family segments. Embo J 3, 1753-1759 (1984).
7 Moran, J. V. & Gilbert, N., Mammalian LINE-1 retrotransposons and related elements. (ASM Press, Washington, D.C., 2002).
8 Kazazian, H. H., Jr. Mobile elements and disease. Curr Opin Genet Dev 8, 343-350 (1998).
9 Han, J. S., Szak, S. T. & Boeke, J. D. Transcriptional disruption by the L1 retrotransposon and implications for mammalian transcriptomes. Nature 429, 268-274 (2004).
10 Perepelitsa-Belancio, V. & Deininger, P. RNA truncation by premature polyadenylation attenuates human mobile element activity. Nat Genet. 35, 363-366 (2003).
11 Muotri, A. R. et al. Somatic mosaicism in neuronal precursor cells mediated by L1 retrotransposition. Nature 435, 903-910 (2005).
12 Muotri, A. R. & Gage, F. H. Generation of neuronal variability and complexity. Nature 441, 1087-1093 (2006).
13 Cao, X. et al. Noncoding RNAs in the Mammalian Central Nervous System. Annu Rev Neurosci (2006).
14 Nan, X. et al. Transcriptional repression by the methyl-CpG-binding protein MeCP2 involves a histone deacetylase complex. Nature 393, 386-389 (1998).
15 Jones, P. L. et al. Methylated DNA and MeCP2 recruit histone deacetylase to repress transcription. Nat Genet. 19, 187-191 (1998).
16 Yu, F., Zingler, N., Schumann, G. & Stratling, W. H. Methyl-CpG-binding protein 2 represses LINE-1 expression and retrotransposition but not Alu transcription. Nucleic Acids Res 29, 4493-4501 (2001).
17 Goodier, J. L., Ostertag, E. M., Du, K. & Kazazian, H. H., Jr. A novel active L1 retrotransposon subfamily in the mouse. Genome Res 11, 1677-1685 (2001).
18 DeBerardinis, R. J., Goodier, J. L., Ostertag, E. M. & Kazazian, H. H., Jr. Rapid amplification of a retrotransposon subfamily is evolving the mouse genome. Nat Genet. 20, 288-290 (1998).
19 Nakashima, K. et al. Synergistic signaling in fetal brain by STAT3-Smad1 complex bridged by p 300. Science 284, 479-482 (1999).
20 Takizawa, T. et al. DNA methylation is a critical cell-intrinsic determinant of astrocyte differentiation in the fetal brain. Dev Cell 1, 749-758 (2001).
21 Amir, R. E. et al. Rett syndrome is caused by mutations in X-linked MECP2, encoding methyl-CpG-binding protein 2. Nat Genet. 23, 185-188 (1999).
22 Amir, R. E. & Zoghbi, H. Y. Rett syndrome: methyl-CpG-binding protein 2 mutations and phenotype-genotype correlations. Am J Med Genet. 97, 147-152 (2000).
23 Wilson, M. & Koopman, P. Matching SOX: partner proteins and co-factors of the SOX family of transcriptional regulators. Curr Opin Genet Dev 12, 441-446 (2002).
24 Esnault, C., Maestre, J. & Heidmann, T. Human LINE retrotransposons generate processed pseudogenes. Nat Genet. 24, 363-367 (2000).
25 Dewannieux, M., Esnault, C. & Heidmann, T. LINE-mediated retrotransposition of marked Alu sequences. Nat Genet. 35, 41-48 (2003).
26 Wei, W. et al. Human L1 retrotransposition: cis preference versus trans complementation. Mol Cell Biol 21, 1429-1439 (2001).
27 Yoder, J. A., Walsh, C. P. & Bestor, T. H. Cytosine methylation and the ecology of intragenomic parasites. Trends Genet. 13, 335-340 (1997).
28 Aravin, A. A., Hannon, G. J. & Brennecke, J. The Piwi-piRNA pathway provides an adaptive defense in the transposon arms race. Science 318, 761-764 (2007).
29 Giacometti, E., Luikenhuis, S., Beard, C. & Jaenisch, R. Partial rescue of MeCP2 deficiency by postnatal activation of MeCP2. Proc Natl Acad Sci USA 104, 1931-1936 (2007).
30 Guy, J. et al. Reversal of neurological defects in a mouse model of Rett syndrome. Science 315, 1143-1147 (2007).
31 Palmer, T. D., Takahashi, J. & Gage, F. H. The adult rat hippocampus contains primordial neural stem cells. Mol Cell Neurosci 8, 389-404 (1997).
32 Gage, F. H., Ray, J. & Fisher, L. J. Isolation, characterization, and use of stem cells from the CNS. Annu Rev Neurosci 18, 159-192 (1995).
33 Chen, R. Z., Akbarian, S., Tudor, M. & Jaenisch, R. Deficiency of methyl-CpG binding protein-2 in CNS neurons results in a Rett-like phenotype in mice. Nat Genet. 27, 327-331 (2001).
34 Muotri, A. R. et al., Somatic mosaicism in neuronal precursor cells mediated by L1 retrotransposition. Nature 435 (7044), 903-910 (2005).
35 Tang, Y., Nyengaard, J. R., De Groot, D. M., & Gundersen, H. J., Total regional and global number of synapses in the human brain neocortex. Synapse (New York, N.Y. 41 (3), 258-273 (2001).
36 Uchida, N. et al., Direct isolation of human central nervous system stem cells. Proc Natl Acad Sci USA 97 (26), 14720-14725 (2000).
37 Brouha, B. et al., Hot L1s account for the bulk of retrotransposition in the human population. Proc Natl Acad Sci USA 100 (9), 5280-5285 (2003).
38 Moran, J. V. et al., High frequency retrotransposition in cultured mammalian cells. Cell 87 (5), 917-927 (1996).
39 Ostertag, E. M. et al., A mouse model of human L1 retrotransposition. Nat Genet. 32 (4), 655-660 (2002).
40 Ostertag, E. M., Prak, E. T., DeBerardinis, R. J., Moran, J. V., & Kazazian, H. H., Jr., Determination of L1 retrotransposition kinetics in cultured cells. Nucleic Acids Res 28 (6), 1418-1423 (2000).
41 Kulpa, D. A. & Moran, J. V., Ribonucleoprotein particle formation is necessary but not sufficient for LINE-1 retrotransposition. Hum Mol Genet. 14 (21), 3237-3248 (2005).
42 Moran, J. & Gilbert, N., Mammalian LINE-1 retrotransposons and related elements. (ASM Press, Washington, D.C., 2002).
43 Myers, J. S. et al., A comprehensive analysis of recently integrated human Ta L1 elements. Am J Hunt Genet. 71 (2), 312-326 (2002).
44 Morrish, T. A. et al., DNA repair mediated by endonuclease-independent LINE-1 retrotransposition. Nat Genet. 31 (2), 159-165 (2002).
45 Gilbert, N., Lutz, S., Morrish, T. A., & Moran, J. V., Multiple fates of L1 retrotransposition intermediates in cultured human cells. Mol Cell Biol 25 (17), 7780-7795 (2005).
46 Symer, D. E. et al., Human II retrotransposition is associated with genetic instability in vivo. Cell 110 (3), 327-338 (2002).
47 Yeo, G. W. et al., Alternative splicing events identified in human embryonic stem cells and neural progenitors. PLoS computational biology 3 (10), 1951-1967 (2007).
48 Bourc'his, D. & Bestor, T. H., Meiotic catastrophe and retrotransposon reactivation in male germ cells lacking Dnmt3L. Nature 431 (7004), 96-99 (2004).
49 Takai, D. & Jones, P. A., The CpG island searcher: a new WWW resource. In Silico Biol 3 (3), 235-240 (2003).
50 Yu, F., Zingler, N., Schumann, G., & Stratling, W. H., Methyl-CpG-binding protein 2 represses LINE-1 expression and retrotransposition but not Alu transcription. Nucleic Acids Res 29 (21), 4493-4501 (2001).
51 Tchenio, T., Casella, J. F., & Heidmann, T., Members of the SRY family regulate the human LINE retrotransposons. Nucleic Acids Res 28 (2), 411-415 (2000).
52 Lee, C., Wevrick, R., Fisher, R. B., Ferguson-Smith, M. A., & Lin, C. C., Human centromeric DNAs. Human genetics 100 (3-4), 291-304 (1997).
53 Pavlicek, A., Paces, J., Zika, R., & Hejnar, J., Length distribution of long interspersed nucleotide elements (LINEs) and processed pseudogenes of human endogenous retroviruses: implications for retrotransposition and pseudogene detection. Gene 300 (1-2), 189-194 (2002).
54 Grimaldi, G., Skowronski, J., & Singer, M. F., Defining the beginning and end of KpnI family segments. Embo J 3 (8), 1753-1759 (1984).
55 Gage, F. H., Mammalian neural stem cells. Science 287 (5457), 1433-1438 (2000).
56 Prak, E. T., Dodson, A. W., Farkash, E. A., & Kazazian, H. H., Jr., Tracking an embryonic L1 retrotransposition event. Proc Natl Acad Sci USA 100 (4), 1832-1837 (2003).
57 Garcia-Perez, J. L. et al., LINE-1 retrotransposition in human embryonic stem cells. Hum Mol Genet. 16 (13), 1569-1577 (2007).
58 van den Hurk, J. A. et al., L1 retrotransposition can occur early in human embryonic development. Hum Mol Genet. 16 (13), 1587-1592 (2007).
59 Thomson, J. A. et al., Embryonic stem cell lines derived from human blastocysts. Science 282 (5391), 1145-1147 (1998).
60 Zhang, S. C., Wernig, M., Duncan, I. D., Brustle, O., & Thomson, J. A., In vitro differentiation of transplantable neural precursors from human embryonic stem cells. Nat Biotechnol 19 (12), 1129-1133 (2001).
61 Forslund, O. et al., Nucleotide sequence and phylogenetic classification of candidate human papilloma virus type 92. Virology 312 (2), 255-260 (2003).
62 Draper, J. S. et al., Recurrent gain of chromosomes 17q and 12 in cultured human embryonic stem cells. Nat Biotechnol 22 (1), 53-54 (2004).
63 Zhao, C., Teng, E. M., Summers, R. G., Jr., Ming, G. L., & Gage, F. H., Distinct morphological stages of dentate granule neuron maturation in the adult mouse hippocampus. J Neurosci 26 (1), 3-11 (2006).
64 Kimberland, M. L. et al., Full-length human L1 insertions retain the capacity for high frequency retrotransposition in cultured cells. Hum Mol Genet. 8 (8), 1557-1560 (1999).
65 Brouha, B. et al., Evidence consistent with human L1 retrotransposition in maternal meiosis I. Am J Hum Genet. 71 (2), 327-336 (2002).
66 Gage, F. H. et al., Survival and differentiation of adult neuronal progenitor cells transplanted to the adult brain. Proc Natl Acad Sci USA 92 (25), 11879-11883 (1995).
67 Athanikar, J. N., Badge, R. M., & Moran, J. V., A YY1-binding site is required for accurate human LINE-1 transcription initiation. Nucleic Acids Res 32 (13), 3846-3855 (2004).
68 Sambrook, J., Fritsch, E., & Maniatis, A., Molecular Cloning: A Laboratory Manual. (Cold Spring Harbor Press, Cold Spring Harbor, 1989).
69 Kent, W. J. et al., The human genome browser at UCSC. Genome Res 12 (6), 996-1006 (2002).

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims.

TABLE 1
Variation of L1 ORF2 sequences in the human brain and heart tissue.

| | | Inverse | | |
|---|---|---|---|---|
| | Individual | ORF2/SATA | Inverse ORF2/SATA | Ratio |
| Phenotype | ID | (Brain) - average | (Heart) - average | B/H |
| Control | 1079 | 1.060562385 | 1.031919974 | +1.03 |
| Control | 1846 | 1.049208156 | 1.034254452 | +1.01 |
| Control | 1347 | 1.015305541 | n/a | — |
| Control | 4786 | 1.072369302 | n/a | — |
| Control | 1571 | 1.057261244 | n/a | — |
| RTT | 1420 | 1.063944818 | 1.026707949 | +1.04 |
| RTT | 4882 | 1.068813016 | 1.026741235 | +1.04 |
| RTT | 1748 | 1.085141193 | n/a | — |
| RTT | 1815 | 1.076675976 | n/a | — |
| RTT | 4852 | 1.045949012 | n/a | — |

TABLE 2
Results of L1 retrotransposition assays in hESC-derived NPCs

| Cell Line | Lab^{a} | Selection^{b} | Plasmid-% EGFP(**) |
|---|---|---|---|
| HUES6 | G | NO | 0.93 +/− 0.11 |
| HUES6 | G | NO | 0.25 +/− 0.04 |
| HUES6 | G | NO | 0.39 +/− 0.03 |
| HUES6 | G | NO | 0.01 +/− 0.02 |
| WA09 (H9) | G | NO | 0.13 +/− 0.01 |
| WA09 (H9) | G | NO | 0.33 +/− 0.06 |
| WA09 (H9) | G | YES | 0.87 +/− 0.39 |
| WA07 (H7) | M | NO | 0.42 +/− 0.2 |
| WA07 (H7) | M | YES | 9.80 +/− 2.82 |
| WA07 (H7) | M | YES | 5.70 +/− 0.46 |
| WA07 (H7) | M | YES | 2.85 +/− 0.86 |
| WA07 (H7) | M | YES | UB 4.65 +/− 0.21 |
| WA13B (H13B) | M | YES | UB 3.25 +/− 0.26 |
| WA13B (H13B) | M | YES | UB 16.25 +/− 3.6 |
| WA13B (H13B) | M | YES | 5.15 +/− 0.54 |
| WA13B (H13B)* | M | YES | 0.82 +/− 0.1 |
| WA13B (H13B)* | M | YES | 0.60 +/− 0.2 |
| WA13B (H13B)* | M | YES | 0.23 +/− 0.05 |
| WA09 (H9) | M | YES | 4.21 +/− 0.84 |
| BG01 | M | YES | 7.73 +/− 1.94 |

TABLE 3
Analysis of L1 insertions in hESC-derived NPCs

| | | Truncation | | | | |
|---|---|---|---|---|---|---|
| NPC^{a} | Analysis^{b} | site^{c} | polyA | EN site | Locus | L1 insertion target site |
| Clone | Full | NA | 47 | 5′-ATTT/TG-3′ | 3p24 | 13 kB upstream from several mRNAs (ESTs), into a LINE. |
| 1 | Full | 6201 | 23 | 5′-TTTT/AT-3′ | 18p11 | 15 kB upstream from Protein APCDD1 precursor |
| | | | | | | (Adenomatosis polyposis coli down-regulated 1 protein) |
| | | | | | | involved in colorectal tumorigenesis |
| 1 | Full | 6229 | 137 | 5′-TCTT/CA-3′ | 7q21 | In an intron of zinc finger protein 804B (ZNF804B) |
| 1 | Full | 6109 | 48 | 5′-AATT/AA-3′ | 2q24 | 100 kB upstream from EST AA319772 |
| 2 | Full | 6133 | 45 | 5′-TTTT/GA-3′ | 10q25 | Into an intron of SLC18A2, a synaptic vesicular monoamine |
| | | | | | | transporter |
| 2 | Full | 6144 | 100 | 5′-TTTT/GG-3′ | 5q21 | 100 kB upstream from EST DA377288, in a LINE element |
| 2 | Full | 5674 | 58 | 5′-TTTC/AC-3′ | 11q24 | 10 kB upstream from several ESTs, in an LTR repeat |
| 2 | Full | 5681 | 110 | 5′-TTTT/AA-3′ | 5p13 | In an exon of C7, complement component 7 precursor (a |
| | | | | | | component of immune complement system) |
| 2 | Full | 6143 | 45 | 5′-TTTT/CG-3′ | 12q13 | 5 KB upstream from olfactory receptor OR6C1, In an intron of |
| | | | | | | EST AK127862 |
| 1 | Partial | NA | 80 | 5′-TTTT/GT-3′ | 7p15 | Intron of pleckstrin homology domain containing family A |
| | | | | | | (PLEKHA8) |
| 1 | Partial | NA | 50 | 5′-TTTT/GT-3′ | 3p14 | 5 kB upstream from PRICKLE 2, prickle-like protein 2 (nuclear |
| | | | | | | membrane protein expressed in brain, eye and testes) |
| 1 | Partial | NA | 98 | 5′-TTCT/GA-3′ | 2q24 | Into an intron of PSMD1: proteasome 26S non-ATPase subunit |
| 1 | Partial | NA | 54 | 5′-TTT/AG-3′ | 3q22 | 7 kB downstream from RYK receptor-like tyrosine kinase |
| | | | | | | isoform 1 (growth factor receptor) |
| 1 | Partial | NA | 72 | 5′-TTTT/AC-3′ | Xq21 | 2.5 kB downstream from GPR174, putative purinergic receptor |
| | | | | | | FKSG79 (G-protein coupled receptor) |
| 1 | Partial | NA | 63 | 5′-ATTT/AT-3′ | 10q23 | 90 kB upstream from NGR3 (Neuregulin 3), Brain expressed |
| | | | | | | direct ligand for the ERBB4 tyrosine kinase receptor |
| 1 | Partial | NA | 139 | 5′-TTTT/AT-3′ | 5p14 | 180 kB downstream from PRDM9 (involved in transcriptional |
| | | | | | | regulation) |
| 1 | Partial | NA | 72 | 5′-ATTT/CT-3′ | 10q24 | Into a region of ESTs of unknown function |
| 1 | Partial | NA | 64 | 5′-TTTT/GG-3′ | 19p13 | 11 kB downstream from KIAA0892 (secreted protein in the |
| | | | | | | mau-2 family) |
| 1 | Partial | NA | 83 | 5′-TTTT/CC-3′ | 16q21 | 70 kB upstream from GOT2, aspartate aminotransferase 2 |
| | | | | | | precursor (role in amino acid metabolism) |

TABLE 4A
Sequences of QPCR ORF2 genomic DNA products

| Sample # | ORF2 match | |
|---|---|---|
| 3H-1 | tgaggaatcgccacactgacttccacaatggttgagctagtttacagtcccaccaacagtgtaaaagtgttcctatttct | SEQ ID NO: 69 |
| | ccgca | |
| 3H-4 | tgaggaatcgccacactgacttccacaatggttgaactagtttacagtcccaccaacagtgtaaaagtgttcctatttct | SEQ ID NO: 70 |
| | ccgca | |
| 3H-5 | tgaggaatcgccacactgacttccacaatggttgaactagtttacagtcccaccaacagtgtaaaagtgttcctatttct | SEQ ID NO: 71 |
| | ccgca | |
| 3H-6 | tgaggaatcgccacactgacttccacaatggttgaactagtttacagtcccaccaacagtgtaaaagtgttcctatttct | SEQ ID NO: 72 |
| | ccgca | |
| 3H-5b | tgaggaatcgccacactgacttccacaatggttgaactagtttacagtcccaccaacagtgtaaaagtgttcctatttct | SEQ ID NO: 73 |
| | ccgca | |
| 3L-9 | tgaggaatcgccacactgacttccaccatggttgaactagtttacagtcccaccaacagtgtaaaagtgttcctatttct | SEQ ID NO: 74 |
| | ccgca | |
| 3L-10 | tgaggaatcgccacactgacttccacaatggttgaactagtttacagtcccatcaacagtgtaaaagtgttcctatttct | SEQ ID NO: 75 |
| | ccgca | |
| 3L-12 | tgaggaatcgccacactgacttccacaatggttgaactagtttacagtcccaccaacagtgtaaaagtgttcctatttct | SEQ ID NO: 76 |
| | ccgca | |
| 3L-13 | tgaggaatcgccacactgacttccacaatggttgaactagtttacagttccaccaacagtgtaaaagtgttcctatttct | SEQ ID NO: 77 |
| | ccgca | |
| 3L-14 | tgaggaatcgccacactgacttccacaatggttgaactagtttacagtcccaccaacagtgtaaaagtgttcctatttct | SEQ ID NO: 78 |
| | ccgca | |
| 3L-15 | tgaggaatcgccacactgacttccacaatggttgaactagtttacagtcccaccaacagtgtaaaagtgttcctatttct | SEQ ID NO: 79 |
| | ccgca | |
| 3L-16 | tgaggaatcgccacactgacttccacaatggttgaactagtttacagtcccaccaacagtgtaaaagtgttcctatttct | SEQ ID NO: 80 |
| | ccgca | |
| 4H-17 | tcaaacaccgcatattctcactcagaggtgggaattgaacaatgagatcacatggacaca | SEQ ID NO: 81 |
| 4H-18 | tcaaacaccgcatattctcactcataggtgggaattgagcaatgagatcacatggacaca | SEQ ID NO: 82 |
| 4H-19 | tcaaacaccgcatattctcactcataggtgggaactggacgatgagatcacatggacacag | SEQ ID NO: 83 |
| 4H-20 | tcaaacaccgcatattctcactcataggtgggaattgaacaatgagatcacatggacaca | SEQ ID NO: 84 |
| 4H-21 | tcaaacaccgcatattctcactcataggtgggaattgaacaatgagatcacatggacaca | SEQ ID NO: 85 |
| 4H-22 | tcaaacaccgcatattctcactcataggtgggaattgaacaatgagatcacatggacacag | SEQ ID NO: 86 |
| 4H-23 | tcaaacaccgcatattctcactcataggtgggaattgaacaatgagatcacatggacaca | SEQ ID NO: 87 |
| 4H-24 | tcaaacaccgcatattctcactcataggtgggaattgaacaatgagatcacatggacacag | SEQ ID NO: 88 |
| 4L-25 | tcaacaccgcatattctcactcataggtgggaattgaacaatgagatcacatggacaca | SEQ ID NO: 89 |
| 4L-26 | tcaacaccgcatattctcactcataggtgggaattgaacaatgagatcacatggacaca | SEQ ID NO: 90 |
| 4L-27 | tcaaacaccgcatattctcactcataggtgggaattgaacaatgagatcacatggacacag | SEQ ID NO: 91 |
| 4L-28 | tcaaaccgcatattctcactcataggtggggattgaacaatgagatcacatggacacag | SEQ ID NO: 92 |
| 4L-29 | tcaacaccgcatattctcactcataggtgggaattgaacaatgagatcacatggacaca | SEQ ID NO: 93 |
| 4L-30 | tcaaacaccgcatattctcactcataggtgggaattgaacaatgagatcacatggacacag | SEQ ID NO: 94 |
| 4L-31 | tcaacaccgcatattctcactcataggtgggaattgaacaatga-atcacatggacacag | SEQ ID NO: 95 |
| 4L-32 | tcaaacaccgcatattctcactcataggtgggaattgaacaatgagatcacatggacacag | SEQ ID NO: 96 |

TABLE 4B
Sequencing analysis of QPCR genomic DNA products

| Sample | ORF2 | | | | |
|---|---|---|---|---|---|
| # | Probe | Tissue | % identity | UCSC | REPEAT M |
| 3H-1 | #1 | Hippocampus | 100 | Hs | L1P1 |
| 3H-4 | #1 | Hippocampus | 100 | Hs | L1P1 |
| 3H-5 | #1 | Hippocampus | 100 | Hs | L1P1 |
| 3H-6 | #1 | Hippocampus | 100 | Hs | L1P1 |
| 3H-5b | #1 | Hippocampus | 100 | Hs | L1P1 |
| 3L-9 | #1 | Liver | 98 | Hs/PA3 | L1P1 |
| 3L-10 | #1 | Liver | 98 | Hs/PA3 | L1P1 |
| 3L-12 | #1 | Liver | 100 | Hs | L1P1 |
| 3L-13 | #1 | Liver | 98 | Hs/PA4 | L1P1 |
| 3L-14 | #1 | Liver | 100 | Hs | L1P1 |
| 3L-15 | #1 | Liver | 100 | Hs | L1P1 |
| 3L-16 | #1 | Liver | 100 | Hs | L1P1 |
| 4H-17 | #2 | Hippocampus | 98 | Hs/PA2/3 | Hs |
| 4H-18 | #2 | Hippocampus | 98 | Hs/PA3 | Hs |
| 4H-19 | #2 | Hippocampus | 95 | PA2/3 | Hs |
| 4H-20 | #2 | Hippocampus | 100 | Hs | Hs |
| 4H-21 | #2 | Hippocampus | 100 | Hs | Hs |
| 4H-22 | #2 | Hippocampus | 100 | Hs | Hs |
| 4H-23 | #2 | Hippocampus | 100 | Hs | Hs |
| 4H-24 | #2 | Hippocampus | 100 | Hs | Hs |
| 4L-25 | #2 | Liver | 100 | Hs | Hs |
| 4L-26 | #2 | Liver | 100 | Hs | Hs |
| 4L-27 | #2 | Liver | 98 | Hs/PA2/3 | Hs |
| 4L-28 | #2 | Liver | 98 | L1PA2 | Hs |
| 4L-29 | #2 | Liver | 100 | Hs | Hs |
| 4L-30 | #2 | Liver | 100 | Hs | Hs |
| 4L-31 | #2 | Liver | 98 | Hs | Hs |
| 4L-32 | #2 | Liver | 100 | Hs | Hs |

TABLE 5A
Sequences of QPCR ORF2 products from RT-PCR (RNA transcripts)

| ID | Sequence | |
|---|---|---|
| 3B | tgaggaatcgccacactgacttccacaatggttgaactagtttacagtcccaccaacagtgtaaaagtgttcctatttctccg | SEQ ID NO: 97 |
| | ca | |
| 3D | tgaggaatcgccacactgacttccacaatggttgaactagtttacagtcccaccaacagtgtaaaagtgttcctatttctccg | SEQ ID NO: 98 |
| | ca | |
| 3H | tgaggaatcgccacactgacttccacaatggttgaactagtttacagtcccatgaactgtgtaaaagtgttcctatttctccg | SEQ ID NO: 99 |
| | ca | |
| 4A | tgaggaatcgccacactgacttccacaatggttgaactagtttacagtcccaccaacagtgtaaaagtgttcctatttctccg | SEQ ID NO: 100 |
| | ca | |
| 4B | tgaggaatcgccacactgacttccacaatggttgaactagtttacagtcccaccaacagtgtaaaagtgttcctatttc | SEQ ID NO: 101 |
| 4G | tgaggaatcnccacactgacttccacaatggttgaactagtttacagtcccaccaacagtgtaaaagtgttcctatttctccg | SEQ ID NO: 102 |
| | ca | |
| 2 | tgaggaatcgccacactgacttccacaatggttgaactagtttacagtcccaccaacagtgtaaaagtgttcctatttctccg | SEQ ID NO: 103 |
| | ca | |
| 4 | tgaggaatcgccacactgacttccacaatggttgaactagtttacagtcccaccaacagggtaaaagtgttcctatttctccg | SEQ ID NO: 104 |
| 5 | tgaggaatcgccacactgacttccacaatggttgaactagtttacagtcccaccaaccgtgtaaaagtgttcctatttctc | SEQ ID NO: 105 |
| hfNSC- | tgaggaatcgccacactgacttccacaatggttgaactagtttacagtcccaccaacagtgtaaaagtgttcctatttctccg | SEQ ID NO: 106 |
| 1 | ca | |
| 7A | tgaggaatcgccacactgacttccacaatggttgaactagttgacagtcccaccaacagtgtaaaagtgttcctatttctccg | SEQ ID NO: 107 |
| 7B | tgaggaatcgccacactgacttccacaatggttgaactagtttacagtcccaccaacagtgtaaaagtgttcctatttctccg | SEQ ID NO: 108 |
| | ca | |
| 7E | tgaggaatcgccacactgacttccacaatggttgaactagttgacagtcccaccaacagtgtaaaagtgttcctatttctccg | SEQ ID NO: 109 |
| | ca | |
| 7G | tgaggaatcgccacactgacttccacaatggttgaactagtttacagtcccaccaacagtgtaaaagtgttcctatttctccg | SEQ ID NO: 110 |
| | ca | |
| 7H | tgaggaatcgccacactgacttccacaatggttgaactagtttacagtcccaccaacagtgtaaaagtgttcctatttctccg | SEQ ID NO: 111 |
| | ca | |
| 8A | tgaggaatcgccacactgacttccacaatggttgaactagtttacagtcccaccaacagtgtaaaagtgttcctatttctccg | SEQ ID NO: 112 |
| | ca | |
| 8B | tgaggaatcgccacactgacttccacaatggttgaactagtttacagtcccaccaacagtgtaaaagtgttcctatttctccg | SEQ ID NO: 113 |
| | ca | |
| 8C | tgaggaatcgccacactgacttccacaatggttgaactagtttacagtcccaccaacagtgtaaaagtgttcctatttctccg | SEQ ID NO: 114 |
| | ca | |
| 8E | tgaggaatcgccacactgacttccacaatggttgaactagtttacagtcccaccaacagtgtaaaagtgttcctatttctccg | SEQ ID NO: 115 |
| | ca | |
| 8H | tgaggaatcgccacactgacttccacaatggttgaactagttgacagtcccaccaacagtgtaaaagtgttcctatttctccg | SEQ ID NO: 116 |
| | ca | |
| 7A-2 | tgaggaatcgccacactgacttccacaatggttgaactagttgacagtcccaccaacagtgtaaaagtgttcctatttctccg | SEQ ID NO: 117 |
| 5D | tgaggaatcgccacactgacttccacaatggttgaactagtttacagtcccaccaacagtgtaaaagtgttcctatttctccg | SEQ ID NO: 118 |
| | ca | |
| 5F | tgaggaatcgccacactgacttccacaatggttgaactagtttacagtcccaccaacagtgtaaaagtgttcctatttctccg | SEQ ID NO: 119 |
| | ca | |
| 6D | tgaggaatcgccacactgacttccacaatggttgaactagtttacagtcccaccaacagtgtaaaagtgttcctatttctccg | SEQ ID NO: 120 |
| | ca | |
| 6F | tgaggaatcgccacactgacttccacaatggttgaactagtttacagtcccaccaacagtgtaaaagtgttcctatttctccg | SEQ ID NO: 121 |
| 6G | tgaggaatcgccacactgacttccacaatggttgaactagtttacagtcccaccaacagtgtaaaagtgttcctatttctccg | SEQ ID NO: 122 |
| | ca | |
| 6H | tgaggaatcgccacactgacttccacaatggttgaactagtttacagtcccaccaacagtgtaaaagtgttcctatttctccg | SEQ ID NO: 123 |
| | ca | |

TABLE 5B
Sequence analysis of QPCR products from L1 RT-PCR

| ID | Sample Type | % Identity | UCSC | Repeat M |
|---|---|---|---|---|
| 3B | fetal brain | 100 | Hs | L1P1 |
| 3D | fetal brain | 100 | Hs | L1P1 |
| 3H | fetal brain | 96 | L1PA4 | L1P1 |
| 4A | fetal brain | 100 | Hs | L1P1 |
| 4B | fetal brain | 100 | Hs | L1P1 |
| 4G | fetal brain | 98 | Hs | L1P1 |
| 2 | fetal brain | 100 | Hs | L1P1 |
| 4 | fetal brain | 97 | Hs | L1P1 |
| 5 | fetal brain | 98 | L1PA3 | L1P1 |
| hfNSC-1 | hCNS-SCns | 100 | Hs | L1P1 |
| 7A | hCNS-SCns | 98 | Hs | L1P1 |
| 7B | hCNS-SCns | 100 | Hs | L1P1 |
| 7E | hCNS-SCns | 98 | Hs | L1P1 |
| 7G | hCNS-SCns | 100 | Hs | L1P1 |
| 7H | hCNS-SCns | 100 | Hs | L1P1 |
| 8A | hCNS-SCns | 100 | Hs | L1P1 |
| 8B | hCNS-SCns | 100 | Hs | L1P1 |
| 8C | hCNS-SCns | 100 | Hs | L1P1 |
| 8E | hCNS-SCns | 100 | Hs | L1P1 |
| 8H | hCNS-SCns | 98 | Hs | L1P1 |
| 7A-2 | hCNS-SCns | 98 | Hs | L1P1 |
| 5D | ES-NPC | 100 | Hs | L1P1 |
| 5F | ES-NPC | 100 | Hs | L1P1 |
| 6D | ES-NPC | 100 | Hs | L1P1 |
| 6F | ES-NPC | 100 | Hs | L1P1 |
| 6G | ES-NPC | 100 | Hs | L1P1 |
| 6H | ES-NPC | 100 | Hs | L1P1 |

TABLE 6A
Actively transcribed ORF1 sequences
Sample

| ID | Sequence |
|---|---|
| 7 | aggaaatacagagaacgccacaaagatactcctcgagaagagcaactccaagacacataattgtcagattcaccaaagttgaaatg |
| SEQ ID NO:126 | aaggaaaaaatgttaagggcagccagagagaaaggtcgggttacccacaaagggaagcccatcagactaacagtggatctctcggc |
| | agaaaccctacaagccagaagagagtgggggccaatattcaacattcttaaagaaaagaattttcaacccagaatttcatatccag |
| | c |
| 14 | aggaaatacagagaacgccacaaagatactcctcgagaagagcaactccatgacacataattgtcagattcaccaaagttgaaatg |
| SEQ ID NO: 127 | aaggaaaaaatgttaagggcagccagagagaaaggtcgggttaccctcaaaggaaagcccatcagactaacagcggatctctcggc |
| | agaaaccctacaagccagaagagagtgggggccaatattcaacattcttaaagaaaagaattttcaacccagaatttcatatccag |
| | c |
| 6 | aggaaatacagagaacgccacaaagatgctcctcgagaagagcaactccaagacacataattgtcagattcaccaaagttgaaatg |
| SEQ ID NO: 128 | aaggaaaaaatgttaagggcagccagagagaaaggtcgggttactcacaaagggaagcccatcagactgacagcggatctctcagc |
| | agaaactctacaagccagaagagagtgggggccaatattcaaaattcttaaagaaaagaattttcaacccagaatttcatatccag |
| | c |
| 9 | aggaaatacagagaacgccacaaagatactcctcgagaagagcaactccaagacacataattgtcagattcaccaaagttgaaatg |
| SEQ ID NO: 129 | aaggaaaaaatgttaagggcagccagagagaaaggtcgggttacccacaaagggaagcccatcagactaacagcagatctcttggc |
| | agaaaccctacaagccagaagagagtgggggccaatattcaacattcttaaagaaaagaattttcaacccagaatttcatatccag |
| | c |
| 13 | aggaaatacagagaacgccacaaagatactcctcaagaagagcaactccaagacacataactgtcagattcaccaaagttgaaatg |
| SEQ ID NO: 130 | aaggaaaaaatgttaagggcagccagagagaaaggtcaggttaccctcaaaggaaagcccatcagactaacagcggatctctcggc |
| | agaaaccctacaagccagaagagagtgggggccaatattcaacattcttaaagacaagaattttcaacccagaatttcatatccag |
| | c |
| 16 | aggaaatacagagaacgccacaaagatactcctcgagaagagcaactccaagacacataattgtcagattcaccgaagttgaaatg |
| SEQ ID NO: 131 | aaggaaaaaatgttaagggcagccagagagaaaggtcgggttaccctcaaagggaagcccatcagactaacagcggatctctcggc |
| | agaaaccctacaagccagaagagag-gggggccaatattcaacattcttaaagaaaagaattttcaacccagaatttcatatccag |
| | c |
| 1 | aggaaatacagagaacgccacaaagatactcctcgagaagagcaactccaagacacataattgtcagattcaccaaagttgaaatg |
| SEQ ID NO: 132 | aaggaaaaaatgttaagggcagccagagagaaaggtcgggttaccctcaaagggaagcccctcagactaacagctgatctctcagc |
| | agaaactctacgagccagaagagagtgggggccaatattcaacattcttaaagaaaagaattttcaacccagaatttcatatccag |
| | c |
| 5 | aggaaatacagagaacgccacaaagatactcctcgagaagagcaactccaagacacataattgtcagattcaccaaagttgaaatg |
| SEQ ID NO: 133 | aaggaaaaaatgttaagggcagccagaga- |
| | aaaggtcaggttacccacaaagggaagcccatcagactaacagtggatctctctgcagaaactctacaagccagaagagagtgggg |
| | gccaatatccaacattcttaaagaaaagaattttcaacccagaatttcatatccagc |
| 4 | aggaaatacagagaacgccacaaagatactcctcgagaagagcaactccaagacacataattgtcagattcaccaaagttgaaatg |
| SEQ ID NO: 134 | aaggaaaaaatgttaagggcagccagagagaaaggtcgggttacccacaaagggaagcccatcagactaacagcggatctctcggc |
| | acaaactctacaagccagaagagaatgggagccaatattaaacattcttaaagaaaagaattttcaacccagaatttcatatccag |
| | c |
| 8 | aggaaatacagagaacgccacaaagatactcctcgagaagagcaactccaagacacataattgtcagattcaccaaagttgaaatg |
| SEQ ID NO: 135 | aaggaaaaaatgttaagggcagccagagagaaagatcggattactcacaaagggaagcccatcagactaacagctgatctctcagc |
| | agaaactctacaagccagaagagagtgggggccaatattcaacattcttaaagaaaagaattttcaacccagaatttcatatccag |
| | c |
| 15 | aggaaatacagagaacgccacaaagacactcctcgagaagagcaactccaagacacataattgtcagattcaccaaagttgaaatg |
| SEQ ID NO: 136 | aaggaaaaaatgttaagggcagccagagagaaaggtcgggttacccacaaagagaagaccatcagactaacagcagatctcttggc |
| | agaaactctacaagccagaagagagtgggggccaatattcaacattcttaaagaaaaggattttcaacccagaatttcatatccag |
| | c |
| 18 | aggaaatacagagaacgccacaaagatactcttcgagaagagcaaccccaagacacgtaattgtcagattcaccaaagttgaaatg |
| SEQ ID NO: 137 | aaggaaaaaatgttaagggcagccagagagaaaggtcgggttaccctcaaagggaagcccatcagactaacagcagatctctctgc |
| | agaaactctacaagccagaagagagtgggggccaatattcaacattcttaaagaaaagaattttcaacccagaatttcatatccag |
| | c |
| 19 | aggaaatacagagaacgccacaaagatactcctcgagaagagcaaccccaagacacataattgtcagattcaccaaagttgaaacg |
| SEQ ID NO: 138 | aaggaaaaaatgttaagggcagccagagagaaaggttgggttacccacaaagggaagcccatcagactaacagcagatctcttggc |
| | agaaaccctacaagccagaagagagtgggggccaatattcaacattcttaaagaaaaggattttcaacccagaatttcatatccga |
| | c |
| 10 | aggaaatacagagaacgccacaaagatactccttgagaagaccaactccaaaacacctaattgtcaaattcaccaaagttgaaatg |
| SEQ ID NO: 139 | aaggaaaaaatgttaagggcagccagagagaaaagtcggcttacccacaaagggaagcccatcagactaacagctgatctcttggc |
| | agaaattctacaagccagaagagagtgggggccaatattcaacattcttaaagaaaagaattttcaacccagaatttcatatccag |
| | c |
| 20 | aggaaatacagagaacgccacaaagatactcttcgagaagagcaaccccaagacacataattgtcagatttgccaaggttgaaatg |
| SEQ ID NO: 140 | aaggaaaaaatgttaagggcagccagagagaaaggtcgggttacccacaaagggaagcccatcagactaacagcggatctctcagc |
| | agaaactctataagccagaaaagagtgggggccaatattcaacattgttaaagaaaataattttcaacccagaatttcatatccag |
| | c |
| 3 | aggaaatacagagaacgccacaaagatgctctttgagaagagtaaccccaagacacataaccatcagattcaccaaggttgaaatg |
| SEQ ID NO: 146 | aaggaaaaaatgttacgggcaaccagagagaaaggctgggttacccacaaatggaagcccatcagact- |
| | acagtggatatctctgcagaaaccctacaagccagaaaagagtgggggccaatattcaacattcttaaagaaaagaattttcaacc |
| | cagaatttcatatccagc |
| 2 | aggaaatacagagaacgccacaaaaatactccttgagaagagcaaccccaaggcacataatcctcagattcaccaacgttgaaatg |
| SEQ ID NO: 147 | aaggaaaaaaatgttgagagcagccagagagaaaggtcgggttacccacaaagagaagcccatcagattaaccatgaatctctctg |
| | cagaaaccctacaagccagaagagagtgggagccaattattcaacattcttaaagaaaataattttcaacccagaatttcatatcc |
| | agc |
| 24 | aggaaatacagagaacgccacaaagatacacctcgagaagagcaaccccaggacacatggttgtcagattcaccaaggttgaaatg |
| SEQ ID NO: 148 | aaggaaagaatgttgagggcggccagagagaaaggtcgggttgcccacgaagggaggtccatcggactaacagcggatctctctgc |
| | agagaccctgcaagccagaagagagtgggggccagtattcaacattcttaaagaaaaagaattttcaacccagaatttcatatcca |
| | gc |
| hfNSC27 | aggaaatacagagaacgccacaaagatactcctcgagaagagcaactccaagacacataattgtcagattcaccaaagttgaaatg |
| SEQ ID NO: 149 | aaggaaaaaatgttaagggcagccagagagaaaggtcgggttaccctcaaaggaaagcccatcagactaacagcggatctctcggc |
| | agaaaccctacaagccagaagagagtgggggccaatattcaacattcttaaagaaaagaattttcaacccagaatttcatatccag |
| | c |
| hfNSC36 | aggaaatacagagaacgccacaaagatactcctcaagaagagcaactccaagacacataattgtcagattcaccaaagttgaaatg |
| SEQ ID NO: 150 | aaggaaaaaatgttaagggcagccagagagaaaggtcgggttaccctcaaagggaagcccatcagactaacagcggatctcttggc |
| | agaaaccctacaagccagaagagagtgggggccaatattcaacattcttaaagaaaagaattttcaacccagaatttcatatccag |
| | c |
| hfNSC35 | aggaaatacngagaacgccacaaacatactcctcgagaagagcaactccaagacacataattgtcagattcaccaaagttgaaatg |
| SSEQ ID NO: 151 | aaggaaaaaatgttaagggcagccagagagaaaggtcgggttacccacaaagggaagcccatcagactaacagcggatctctcggc |
| | agaaaccctacaagccagaagagggtgggggccaatattcaacattcttaaagaaaagaattttcaacccagaatttcatatccag |
| | c |
| hfNSC17 | gctggatatgaaattctgggttgaaaattcttttctttaagaatgttgaatattggcccccactctcttctggcttgtagggtttt |
| SEQ ID NO: 152 | ggccgagagatcagctgttagtctgatgggcttccctttgtgggtaacccaacctttctctctggctgcccttaacattttttcct |
| | tcatttcaactttggtgaatctgacaattatgtgtcttggagttgctcttctccaggagtatctttgtggcgttctctgtatttcc |
| | t |
| hfnSC25 | aggaaatacagagaacgccacaaaaatactcctcgagaagagcaactccaagacacataattgtcagattcaccaaagttgaaatg |
| SEQ ID NO: 153 | aaggaaaaaatgttaagggcagccagagagacatgtcgggttaccctcaaagggaagcccatcacactaacagcggatctctctgc |
| | agaaacgctacaagccagaagagagtgggggccaatattcaacattcttaaagaaaagaattttcaacccagaatttcatatccag |
| | c |
| hfnSC18 | gctggatatgaaattctgggttgaaaattcttttctataagaacgttgaatattggcccccactctctcctggcttgtagggtttc |
| SEQ ID NO: 154 | tgccaagagatccactgttagtctgatgagcttccctttgtaggtaacccaacctttctctctggctgcccttaacattttttcct |
| | tcatttcaaccttggtgaatctgacaattatgtgtcttggggttgctcttctcaaggagtatctttgtggcgttctctgtatttcc |
| | t |
| hfnSC19 | aggaaatacagagaacgccacaaagatactcctcgagaagaggaatcccaagacacataatcatcagatccaccaaggttgaaatg |
| SEQ ID NO: 155 | aaggaaaaaatgttaagggcagccagagagaaaggtcgggttacccacaaagggaagcccatcagactaacagtggatctctcggg |
| | agaaactctacaagccagaagagagtgggggccaatattcgacattcttaaagaaaagaattttcaacccagaatttcatatccag |
| | c |
| hfNSC20 | gctggatatgaaattctgggttgaaaattcttttctataagaacgttgaatattggcccccactctctcctggcttgtagggtttc |
| SEQ ID NO: 156 | tgccaagagatccactgttagtctgatgagcttccctttgtaggtaacccaacctttctctctggctgcccttaacattttttcct |
| | tcatttcaaccttggtgaatctgacaattatgtgtcttggggttgctcttctcaaggagtatctttgtggcgttctctgtatttcc |
| | t |
| hfNSC23 | aggaaatacagagaacgccacaaagatactcctcgagaagaggaatcccaagacacataatcatcagatccaccaaggttgaaatg |
| SEQ ID NO: 157 | aaggaaaaaatgttaagggcagccagagagaaaggtcgggttacccacaaagggaagcccatcagactaacagtggatctctcggg |
| | agaaactctacaagccagaagagagtgggggccaatattcgacattcttaaagaaaagaattttcaacccagaatttcatatccag |
| | c |
| hfNSC24 | gctggatatgaaattctgggttgaaaattcttttctataagaacgttgaatattggcccccactctctcctggcttgtagggtttc |
| SEQ ID NO: 158 | tgccaagagatccactgttagtctgatgagcttccctttgtaggtaacccaacctttctctctggctgcccttaacattttttcct |
| | tcatttcaaccttggtgaatctgacaattatgtgtcttggggttgctcttctcaaggagtatctttgtggcgttctctgtatttcc |
| | t |
| hfNSC28 | gctggatatgaaattctgggttgaaaattcttttctataagaacgttgaatattggcccccactctctcctggcttgtagggtttc |
| SEQ ID NO: 159 | tgccaagagatccactgttagtctgatgagcttccctttgtaggtaacccaacctttctctctggctgcccttaacattttttcct |
| | tcatttcaaccttggtgaatctgacaattatgtgtcttggggttgctcttctcaaggagtatctttgtggcgttctctgtatttcc |
| | t |
| hfNSC32 | gctggatatgaaattctgggttgaaaattcttttctataagaacgttgaatattggcccccactctctcctggcttgtagggtttc |
| SEQ ID NO: 160 | tgccaagagatccactgttagtctgatgagcttccctttgtaggtaacccaacctttctctctggctgcccttaacattttttcct |
| | tcatttcaaccttggtgaatctgacaattatgtgtcttggggttgctcttctcaaggagtatctttgtggcgttctctgtatttcc |
| | t |
| hfNSC22 | aggaaatacagagaacgccacaaagatactcctcgagaagagcaactccaagatacataaaggaaatacagagaacgccacaaaga |
| SEQ ID NO: 161 | tactcctcgagaagagcaactccaagatacataagttggattacccacaaagggaagcccatcagactaacagctgatctctcggc |
| | agaaactctataagccagaagagtg-gggggccaatattcaacattcttaaagaaaagaattttcaacccagaatttcatatccag |
| | c |
| hfNSC30 | gctggatatgaaattctgggttgaaaattcttttctttcagaatgttgaatattggaccccactctcttctggcttgtagagtttc |
| SEQ ID NO: 162 | tgccgagagatccactgttaagtctgatgggcttccctttgcaggtaacctgacctttctctctggctgcccttaatatgttttcc |
| | ttcatttcaactttggtgaatctgacaatttatgtgtcttggagttgctcttctcgaggtgttacctttgtggcgttctctgtatt |
| | tcct |
| hfNSC31 | aggaaatacagagaacgccacaaagatactcctcgagaagagcaactccaagatacataattgtcagattcaccaaagttgaaatg |
| SEQ ID NO: 163 | aa-gaaaaaatgttaagggccgccagagagaaaggttggattacccacaaagggaagcccatcagactaacagctgatctctcggc |
| | agaaactctataagccagaagagtg-gggggccaatattcaacattcttaaagaaaagaattttcaacccagaatttcatatccag |
| | c |
| hfNSC34 | aggaaatacagagaacgccacaaagatactcctcaagaagagcaaccccaagacgcataattgtcagattcaccaaagttgaaa- |
| SEQ ID NO: 164 | gaaggaaaaaatgttaagggcagccagagagaaaggtcaggttgcccacaaagggaagcccatcggactaacagtggatccctaag |
| | cagaaactctacaagccagaagacagtgggggccaatattcaacattcttaaagaaaagaattttcaacccagaatttcatatcca |
| | gc |
| hfNSC26 | aggaaatacagagaacgccacaaagatattcctcgagaagagcaaccccaagacacataatcatcagattaaccaaggttgaaatg |
| SEQ ID NO: 165 | aaggaaaaaatgttaagggcagccagagagaaaggtcgggttacccacaaagggaagcccatcagactaacagcagatctcttgga |
| | agaaaccctataagccagaagacagtgggggccaatattcaacattcttaaag---agaattttcaacccagaatttcatatccag |
| | c |
| hfNSC21 | gctggatatgaaattctgggttgaaaattcttttctttaagaatgttgaatattagcccccaatctcttgtggcttttagggtttc |
| SEQ ID NO: 166 | tgcagagagatctgctgttagtatgatgggcttccctttgtaggtaacccaacttttctctctggctgcccttaacattttttctt |
| | tcagttcaaccttggtgaatctgacgattatgtgtcttggggttgctcttctcaaagagtatctttgtggcgttctctgtatttcc |
| | t |
| hfNSC29 | aggaaatacagagaacgccacaaagatactcctcgagaagagcaaccccaagacacataatcatcagattcaccaaggttgaaatg |
| SEQ ID NO: 167 | aaggaaaaaatgttaagggcagccagagagaaaggtcacattacccacaaagggaaacccatcagactaacggcggatctctctgc |
| | agaaacgctacaacccagaaaagagtgggggcacacatttaacattcttaaggaaaagaattttcaacccagaatttcatatccag |
| | c |
| hfNSC33 | aggaaatacagagaacgccacaaagatactccttgagaagagcaaccccaagacacataattgtcagattcaccaaggttgaaatg |
| SEQ ID NO: 168 | aaggaaaaagtgttaagggcagccaaagagaaaggttgagttacccacaaagggacgcccatcagactaacagtggatctctctgc |
| | agaaaccctaca----agaagagagtgggggccaatattcaacattctt-----aaagaattttcaacccagaatttcatatccag |
| | c |
| NPC-11 | aggaaatacagagaacgccacaaagatactcctcgagaagagcaactccaagacacataattgtcagattcaccaaagttgaaatg |
| SEQ ID NO: 169 | aaggaaaaaatgttaagggcagccagagagaaaggtcgggttaccctcaaaggaaagcccatcagactaacagcggatctctcggc |
| | agaaaccctacaagccagaagagagtgggggccaatattcaacattcttaaagaaaagaattttcaacccagaatttcatatccag |
| | c |
| NPC-1F | aggaaatacagagaacgccacaaagatactcctcgagaagagcaactccaagacacataattgtcagattcaccaaagttgaaatg |
| SEQ ID NO: 170 | aaggaaaaaatgttaagggcagccagagagaaaggtcgggttaccctcaaaggaaagcccatcagactaacagcggatctctcggc |
| | agaaaccctacaagccagaagagagtgggggccaatattcaacattcttaaagaaaagaattttcaacccagaatttcatatccag |
| | c |
| NPC-2C | aggaaatacagagaacgccacaaagatactcctcgagaagagcaactccaagacacataattgtcagattcaccaaagttgaaatg |
| SEQ ID NO: 171 | aaggaaaaaatgttaagggcagccagagagaaaggtcgggttaccctcaaaggaaagcccatcagactaacagcggatctctcggc |
| | agaaaccctacaagccagaagagagtgggggccaatattcaacattcttaaagaaaagaattttcaacccagaatttcatatccag |
| | c |
| NPC37 | aggaaatacagagaacgccacaaagatactcctcgagaagagcaactccaagacacataaatgtcagattcaccaaagttgaaatg |
| SEQ ID NO: 172 | aaggaaaaaatgttaagggcagccagagagaaaggtcgggttaccctcaaagggaagcccatcagactaacagcggatctctcggc |
| | agaaaccctacaagccagaagagagtgggggccaatattcaacattcttaaagaaaagaattttcaacccagaatttcatatccag |
| | c |
| NPC-1C | aggaaatacagagaacgccacaaagatactcctcgagaagagcaactccaagacacataattgtcagattcaccaaagttgaaatg |
| SEQ ID NO: 173 | aaggaaaaaatgttaagggcagccagagagaaaggtcgggttaccctcaaagggaagcccatcagactaacagcggatctcttggc |
| | agaaactctacaagccagaagatagtgggggccaatattcaacattcttaaagaaaagaattttcaacccagaatttcatatccag |
| | c |
| NPC-1D | aggaaatacnganaacgccacaaagatactcctcgagaagagcaactccaagacacataattgtcagattcaccaaagttgaaatg |
| SEQ ID NO: 174 | aaggaaaaaatgttaagggcagccagagagaaaggtcgggttaccctcaaaggaaagcccatcagactaacagcggatctctcggc |
| | agaaaccctacaagccagaagagagtgggggccaatattcaacattcttaaagaaaagaattttcaacccagaatttcatatccag |
| | c |
| NPC-1H | aggaaatacngagaacgccacaaagatactcctcgagaagagcaactccaagacacataattgtcagattcaccaaagttgaaatg |
| SEQ ID NO: 175 | aaggaaaaaatgttaagggcagccagagagaaaggtcgggttaccctcaaagggaagcccatcagactaacaacggatctctcggc |
| | agaaaccctacaagccagaagagagtgggggccaatattcaacattcttaaagaaaagaattttcaacccagaatttcatatccag |
| | c |
| NPC-2D | aggaaatacagagaacgccacaaagatactcctcgagaagagcaactccaagacacataattgtcagattcaccaaagttgaaatg |
| SEQ ID NO: 176 | aaggaaaaaatgttaagggcagccagagagaaaggtcgggttactctcaaagggaagcccatcagactaacagcggatctctcggc |
| | agaaaccctacaagccagaagagagtgggggccaatattcaacattcttaaagaaaagaattttcaaccagaatttcatatccagc |
| NPC-1A | gaaatactcannacgccacaaagatactcctcgagaagagcaactcaaagacacataattgtcagattcaccaaagttgaaatgaa |
| SEQ ID NO: 177 | ggaaaaaatgttaagggcagccagagagaaaggtcgggttaccctctaagggaagcccatcagactaacagcggatctctcggcag |
| | aaaccctacaaaccagaagagagagtgggggccaatattcaacattcttaaagaaaagaattttcaacccagaatttcatatccag |
| | c |
| NPC-2A | aggaaatacagagaacgccacaaagatactcctcgagaagggcaactgcaagacacataattgtcagattcaccaaagttgaaatg |
| SEQ ID NO: 178 | aaggaaaaaatgttaagggcagccagagagaaaggtcgggttcccctcaaagggaagcccatcagactaacagcggttctctcggc |
| | agaaaccccacaagccagaagacagtggggaccaatattcaacattcttaaagaaaagaattttcaacccagaatttcatatccag |
| | c |
| NPC-2F | aggaaatacagagaacgccacaaagatactcttcgagaagagcaaccccaagacacataattgtcagattcaccaaggttgaaagg |
| SEQ ID NO: 179 | aaggaaaaaatgttaagggcagccagagagaaagtacaagccagaagagagtgggggccaatattcaacattcttaatgaaaagaa |
| | ttttcaacccagaatttcatatccagc |
| NPC-2E | aggaaatacagagagaacgccacaaagatactcctcgagaagagcaactccaagacacataatcgtcagattcaccaaggttgaaa |
| SEQ ID NO: 180 | tgaaggaaaaaatgttaaggattaccagagagaaaggtcgggttccccacaaaggaaagcccatcagactaacagcggatctctcg |
| | gcagaaactctacaagccagaagagagtgggggccaatattcagcattcttaaagaaaagaattttcaacccagaatttcatatcc |
| | agc |
| NPC-12 | aggaaatacagagaacgccacaaagatactccatgagaagagtaaccccaagaaatataattgtcagattcaccaaggttgaaatg |
| SEQ ID NO: 181 | aaggaaaaaatgttaagggcagccagagagaaaggtcgggttacccacaaagggaagcccatcagactaaaagcagatctctcggc |
| | agaaaccctataagccagaaaagagtgggggccaatattcaacattcttaaagaaatgaattttcaacccagaatttcatatccag |
| | c |
| NPC-13 | aggaaatacagagaacgccacaaagatagtccttgagaagagcaactccaagacacataattgtcagattcaccaaagttgaaatg |
| SEQ ID NO: 182 | aagaaacaaatgttcagggcagccagagagaaaggtcaggttacccacaaagggaagcccatcagactaacagctgatctctctgc |
| | agaaactctaccagccagaagagagtggggaccaatattcaacattcttaaagaaaagaattttcaacccagaatttcatatccag |
| | c |
| NPC-14 | aggaaatacagagaacgccacaaagatactccatgagaagagtaaccccaagaaatataattgtcagattcaccaaggttgaaatg |
| SEQ ID NO: 183 | aaggaaaaaatgttaagggcagccagagagaaaggtcgggttacccacaaagggaagcccatcagactaaaagcagatctctcggc |
| | agaaaccctataagccagaaaagagtgggggccaatattcaacattcttaaagaaatgaattttcaacccagaatttcatatccag |
| | c |
| NPC-16 | aggaaatacagagaacgccacaaagatactccatgagaagagtaaccccaagaaatataattgtcagattcaccaaggttgaaatg |
| SEQ ID NO: 184 | aaggaaaaaatgttaagggcagccagagagaaaggtcgggttacccacaaagggaagcccatcagactaaaagcagatctctcggc |
| | agaaaccctataagccagaaaagagtgggggccaatattcaacattcttaaagaaatgaattttcaacccagaatttcatatccag |
| | c |
| NPC-1E | gctggatatgaaattctgggttgaaaattcttttctttaagaatgttgaatattggcccccactctcttctggtttgtagggtttc |
| SEQ ID NO: 185 | tgcaaagagatccgctgttagtctgatgggcttctctttgtgggtaacccgacctttctctctggctgcccttaacattttttcct |
| | tcatttcaaccttggtgaatctgacaattatttgtcttggggttgct-gtctcgaagagtgtctttgtggcgttct-tgtatttcc |
| | t |
| NPC-1G | gctggatatgaaattctgggttgaaaattcttttctttaagaatgttgaatactggcccccactctcttctggattggag-- |
| SEQ ID NO: 186 | tttctgctgagagatcagttgttagtctgatgggcttccctttgtgggtaacccgacctttctctctggctgcccttaacattttc |
| | tccttcatttcaactttggtgaatctgacaattatgtgtcttggagttgctcttctcgaggagcatctttgtggcgttctctgtat |
| | ttcct |
| NPC-2B | aggaaatacagagaacgccacaaagatactccctgcgaagagcaaccccaagacacataatcgtcagattc- |
| SEQ ID NO: 187 | ccaaggttgaaatgaaggaaaaaatgttaagggcagccaaagagaaaggttggattacccccaaagggacgcccattgaactaaca |
| | gcagatctctctgcaaaaaccctacaagccagaatagagtgggggccaatattcaacattcttaaagaaaagaa-tttcaacccag |
| | aatttcatatccagc |
| NPC-2G | aggaaatacagagaacgccacaaagatactccttgcgaagagcaaccccaagacacataatcgtcagattc- |
| SEQ ID NO: 188 | ccaaggttgaaatgaaggaaaaaatgttaagggcagccaaagagaaaggttggattacccccaaagggacgcccattgaactaaca |
| | gcagatctctctgcaaaaaccctacaagccagaatagagtgggggccaatattcaacattcttaaagaaaagaa-tttcaacccag |
| | aatttcatatccagc |

TABLE 6B
Sequence analysis of actively transcribed ORF-1 fragments from RT-PCR

| Sample | | | | | |
|---|---|---|---|---|---|
| ID | Sample | | UCSC | Repeat M | Genomic Location |
| 7 | Fetal brain | | L1PA3 | Hs | 4 kB downstream from interleukin 1 receptor |
| | | | | | accessory protein-like 1 (IL1RAPL1) |
| 14 | Fetal brain | | Hs | Hs | In an intron of Homo sapiens protocadherin |
| | | | | | 11 Y-linked (PCDH11Y) |
| 6 | Fetal brain | | L1PA5 | L1P1 | 6 kB upstream from Homo sapiens dynein, |
| | | | | | axonemal, intermediate chain 1 (DNAI1) |
| 9 | Fetal brain | | L1PA2 | Hs | 10 kB downstream from Homo sapiens |
| | | | | | transmembrane protein with EGF-like and |
| | | | | | two follistatin-like domains 1 (TMEFF1) |
| 13 | Fetal brain | | L1PA2 | Hs | In an intron of Homo sapiens solute carrier |
| | | | | | family 24 (sodium/potassium/calcium |
| | | | | | exchanger), member 2 (SLC24A2) |
| 16 | Fetal brain | | L1PA2 | Hs | In an intron of Homo sapiens astrotactin 2 |
| | | | | | (ASTN2) |
| 1 | Fetal brain | | L1PA3 | L1P1 | no genes within 50 kB |
| 5 | Fetal brain | | L1PA5 | L1P1 | In an intron of Homo sapiens paralemmin 2 |
| | | | | | (PALM2) |
| 4 | Fetal brain | | L1PA3 | L1P1 | In an intron of Homo sapiens odz, odd |
| | | | | | Oz/ten-m homolog 3 (Drosophila) (ODZ3) |
| 8 | Fetal brain | | L1PA4 | L1P1 | In an intron of methyltransferase like 6 |
| 15 | Fetal brain | | L1P1 | L1P1 | In an intron of Homo sapiens catenin |
| | | | | | (cadherin-associated protein), alpha 3 |
| | | | | | (CTNNA3) |
| 18 | Fetal brain | | L1PA3 | Hs | In an intron of SEC14 and spectrin domains 1 |
| 19 | Fetal brain | 96 | L1PA3 | L1P2 | In an intron of Homo sapiens lipoma |
| | | | | | HMGIC fusion partner-like 2 (LHFPL2) |
| 10 | Fetal brain | 94 | L1PA4 | L1P1 | In an intron of Homo sapiens leucine rich |
| | | | | | repeat transmembrane neuronal 4 |
| | | | | | (LRRTM4) |
| 20 | Fetal brain | 94 | L1P2 | L1P2 | 4 kB downstream from Homo sapiens |
| | | | | | dishevelled associated activator of |
| | | | | | morphogenesis 1 (DAAM1) |
| 21 | Fetal brain | 94 | L1PA4 | L1P1 | In an intron of Homo sapiens mirror-image |
| | | | | | polydactyly 1 (MIPOL1) |
| 22 | Fetal brain | 94 | L1PA7 | L1P2 | In an intron of Homo sapiens F-box and |
| | | | | | leucine-rich repeat protein 17 (FBXL17) |
| 23 | Fetal brain | 94 | L1PA5 | L1P1 | no genes within 50 kB |
| 12 | Fetal brain | 93 | L1P2 | L1P2 | Within an intron of Homo sapiens lipoma |
| | | | | | HMGIC fusion partner-like 3 (LHFPL3) |
| 17 | Fetal brain | 93 | L1P2 | L1P2 | Within an intron of Homo sapiens lipoma |
| | | | | | HMGIC fusion partner-like 3 (LHFPL3) |
| 3 | Fetal brain | 92 | L1P2 | L1P2 | Within an intron of Homo sapiens dipeptidyl- |
| | | | | | peptidase 10 (DPP10) |
| 2 | Fetal brain | 91 | L1PA7 | L1P2 | no genes within 50 kB |
| 24 | Fetal brain | 91 | L1PA6 | L1P2 | In an intron of Homo sapiens neuronal cell |
| | | | | | adhesion molecule (NRCAM) |
| hfNSC27 | hCNS-SCns | 99 | Hs | Hs | In an intron of Homo sapiens protocadherin |
| | | | | | 11 Y-linked(PCDH11Y) |
| hfNSC36 | hCNS-SCns | 98 | L1PA3 | Hs | Into a region of antibody parts |
| hfNSC35 | hCNS-SCns | 97 | Hs | Hs | 5 kB downstream from hepatocyte nuclear |
| | | | | | factor 4, gamma |
| hfNSC17 | hCNS-SCns | 96 | L1PA4 | L1P1 | 8 kB downstream from proteolipid protein 1 |
| hfNSC25 | hCNS-SCns | 96 | L1PA4 | Hs | Into an intron of Homo sapiens cDNA clone |
| | | | | | IMAGE: 5298883 |
| hfNSC18 | hCNS-SCns | 95 | L1PA6 | L1P2 | Into an intron of cyclin-dependent kinase- |
| | | | | | like 3 |
| hfNSC19 | hCNS-SCns | 95 | L1PA3 | L1P2 | 10 kB downstream from glycosyltransferase S |
| hfNSC20 | hCNS-SCns | 95 | L1PA6 | L1P2 | Into an intron of cyclin-dependent kinase- |
| | | | | | like 3 |
| hfNSC23 | hCNS-SCns | 95 | PA3 | L1P2 | 10 kB downstream from glycosyltransferase 8 |
| hfNSC24 | hCNS-SCns | 95 | PA6 | L1P2 | Into an intron of cyclin-dependent kinase- |
| | | | | | like 3 |
| hfNSC28 | hCNS-SCns | 95 | PA6 | L1P2 | Into an intron of cyclin-dependent kinase- |
| | | | | | like 3 |
| hfNSC32 | hCNS-SCns | 95 | PA6 | L1P2 | Into an intron of cyclin-dependent kinase- |
| | | | | | like 3 |
| hfNSC22 | hCNS-SCns | 94 | PA4 | L1P1 | no genes within 50 kB |
| hfNSC30 | hCNS-SCns | 94 | PA2 | Hs | no genes within 50 kB |
| hfNSC31 | hCNS-SCns | 94 | PA4 | L1P1 | no genes within 50 kB |
| hfNSC21 | hCNS-SCns | 92 | L1PA7 | L1P2 | In an intron of Na+/K+ transporting ATPase |
| | | | | | interacting 3 |
| hfNSC29 | hCNS-SCns | 91 | L1PA7 | L1P2 | In an intron of DCC-interacting protein 13- |
| | | | | | beta (Dip13-beta) |
| hfNSC33 | hCNS-SCns | | L1PA6 | L1P2 | In an intron of Homo sapiens cDNA |
| | | | | | FLJ30851 fis, clone FEBRA2002908 |
| NPC-11 | ES-NPC | 99 | Hs | Hs | In an intron of protocadherin 11 Y-linked |
| NPC-1F | ES-NPC | 99 | Hs | Hs | In an intron of protocadherin 11 Y-linked |
| NPC-2C | ES-NPC | 99 | Hs | Hs | In an intron of protocadherin 11 Y-linked |
| NPC37 | ES-NPC | 98 | L1PA2 | Hs | no genes within 50 kB |
| NPC-1C | ES-NPC | 98 | L1PA2 | Hs | 5 kB downstream from Homo sapiens |
| | | | | | upstream binding transcription factor, RNA |
| | | | | | polymerase I-like 1 (UBTFL1) |
| NPC-1D | ES-NPC | 98 | Hs | Hs | In an intron of mitochondrial solute carrier |
| | | | | | protein SLC25A43. |
| NPC-1H | ES-NPC | 98 | L1PA2 | Hs | In an intron of astrotactin 2 isoform b |
| NPC-2D | ES-NPC | 98 | L1PA2 | Hs | no genes within 50 kB |
| NPC-1A | ES-NPC | 96 | L1PA2 | Hs | 10 kB upstream from defensin, beta 126 |
| | | | | | preproprotein |
| NPC-2A | ES-NPC | 96 | L1PA2 | Hs | In an intron of diacylglycerol kinase, beta |
| | | | | | isoform 1 |
| NPC-2F | ES-NPC | 96 | L1PA6 | L1P2 | In an intron of Helicase ARIP4 (EC 3.6.1.—) |
| | | | | | (Androgen receptor-interacting protein 4) |
| | | | | | (RAD54-like protein 2) |
| hfNSC34 | hCNS-SCns | 94 | L1PA5 | L1P2 | In an intron of coiled-coil domain containing |
| | | | | | 66 |
| hfNSC26 | hCNS-SCns | 93 | L1P2 | L1P2 | In an intron of Dmx-like 2 |
| NPC-2E | ES-NPC | 95 | L1PA5 | Hs | 8 kB downstream of dynein, axonemal, |
| | | | | | intermediate chain 1 |
| NPC-12 | ES-NPC | 94 | L1PA6 | L1P2 | no genes within 50 kB |
| NPC-13 | ES-NPC | 94 | L1PA4 | L1P1 | 10 kB upstream from ATPase family, AAA |
| | | | | | domain containing 2B |
| NPC-14 | ES-NPC | 94 | L1PA6 | L1P2 | no genes within 50 kB |
| NPC-16 | ES-NPC | 94 | L1PA6 | L1P2 | no genes within 50 kB |
| NPC-1E | ES-NPC | 94 | L1PA7 | L1P2 | In an intron of oxidation resistance 1 |
| | | | | | isoform 2 |
| NPC-1G | ES-NPC | 94 | L1PA4 | L1P1 | In an intron of ALMS1 |
| NPC-2B | ES-NPC | 91 | L1P2 | L1P2 | In an intron of NIMA (never in mitosis gene |
| | | | | | a)-related kinase |
| NPC-2G | ES-NPC | 91 | L1P2 | L1P2 | In an intron of NIMA (never in mitosis gene |
| | | | | | a)-related kinase |

What is claimed is:

1. A method of treating non-LTR retrotransposition in neural cells, the method comprising exposing a neural cell to a transposition inhibitor in an amount sufficient to decrease non-LTR retrotransposition in the neural cell or a progeny of the neural cell.

2. The method of claim 1, wherein the non-LTR retrotransposition involves at least one L1 retrotransposon.

3. The method of claim 1, wherein the neural cell is a neural stem cell or a neural precursor cell.

4. The method of claim 1, wherein the neural cell is a mammalian cell.

5. The method of claim 5, wherein the neural cell is a human cell.

6. The method of claim 1, wherein the neural cell is identified with a nervous system condition resulting from non-LTR retrotransposition in neural cells.

7. The method of claim 6, wherein the nervous system condition is autism or autism spectrum disorders, schizophrenia, Rett syndrome, Tourette syndrome, ataxia telangiectasia and other ataxias, xeroderma pigmentosum, Cockyne syndrome, fragile x, aspergers syndrome, childhood disintegrative disorder, tuberous sclerosis complex, or neurogiromatosis, Prader-Willi, Angelman, Joubert, Down, Williams and Cowdern syndrome or other psychiatric disorders, or any combination of conditions thereof.

8. The method of claim 1, wherein the transposition inhibitor is an anti-retroviral drug; an inhibitor of RNA stability; an inhibitor of reverse transcription; an inhibitor of L1 endonuclease activity; a stimulator of DNA repair machinery; a zinc-finger that targets the L1 promoter region; an enzyme that inhibits L1; a repressor that inhibits L1; or any combination thereof.

9. The method of claim 1, wherein the neural cell is a fetal or embryonic cell.

10. The method of claim 1, wherein the neural cell is in a patient.

11. The method of claim 10, wherein the neural cell is in an embryo or fetus in the patient.

12. A method of assaying retrotransposition in neural cells, comprising:
a) sorting synchronized neural cells of the same genetic background into single neural cells; and
b) subjecting one or more of the sorted single neural cells to quantitative polymerase chain reaction amplification of at least one retrotransposon.

13. The method of claim 12, further comprising comparing the content of the at least one retrotransposon in the one or more of the sorted single neural cells to the content of the at least one retrotransposon in one or more control cells.

14. The method of claim 13, wherein the one or more control cells is a neural or non-neural cell of comparable genetic background to the synchronized neural cells.

15. The method of claim 12, wherein the at least one retrotransposon is a non-LTR retrotransposon.

16. The method of claim 15, wherein the non-LTR retrotransposon is an L1 retrotransposon.

17. The method of claim 12, wherein the neural cell is a neural stem cell or a neural precursor cell.

18. A method of identifying an inhibitor of retrotransposition, comprising:
a) exposing one or more neural precursor cells to a candidate inhibitor;
b) determining the content of at least one retrotransposon in the one or more neural precursor cells, or in progeny of the one or more neural precursor cells, or in both neural precursor and progeny cells; and
c) comparing the content of the at least one retrotransposon in the one or more neural precursor cells, or in their progeny, or both, to the content of the at least one retrotransposon in one or more control cells not exposed to the candidate inhibitor,
wherein a decrease in content of the at least one retrotransposon in the one or more neural precursor cells, or in their progeny, or both, compared to the one or more control cells is indicative of inhibition of retrotransposition.

19. The method of claim 18, wherein the at least one retrotransposon is a non-LTR retrotransposon.

20. The method of claim 19, wherein the non-LTR retrotransposon is an L1 retrotransposon.

21. The method of claim 18, wherein the one or more control cells are neural precursor cells, or their progeny, or both.

22. A method of identifying a neural condition associated with non-LTR retrotransposition, comprising determining the content of at least one non-LTR retrotransposon in a neural cell in comparison to the content of the at least one non-LTR retrotransposon in one or more control cells, wherein the neural cell comprises a genotype associated with a nervous system condition.

23. The method of claim 22, wherein the nervous system condition is autism or autism spectrum disorders, schizophrenia, Rett syndrome, Tourette syndrome, ataxia telangiectasia and other ataxias, xeroderma pigmentosum, Cockyne syndrome, fragile x, aspergers syndrome, childhood disintegrative disorder, tuberous sclerosis complex, or neurogiromatosis, Prader-Willi, Angelman, Joubert, Down, Williams and Cowdern syndrome or other psychiatric disorders, or any combination of conditions thereof.

24. The method of claim 22, wherein the neural cell is from a knockout animal.

25. The method of claim 22, wherein the neural cell is from an individual having the nervous system condition.

26. The method of claim 22, wherein the at least one retrotransposon is a non-LTR retrotransposon.

27. The method of claim 26, wherein the non-LTR retrotransposon is an L1 retrotransposon.

28. A method of measuring Line-1 retrotransposition activity in single cells comprising:
(i) separating a tissue into single cells;
(ii) isolating genomic DNA from said single cells, thereby forming single cell DNA samples;
(iii) incubating said single cell DNA samples with Line-1 primers and control primers;
(iv) amplifying a Line-1 DNA with said Line-1 primers, thereby forming an amplified Line-1 DNA;
(v) amplifying a control DNA with said control primers, thereby forming an amplified control DNA;
(vi) comparing an amount of said amplified Line-1 DNA with an amount of said amplified control DNA, thereby measuring Line-1 retrotransposition activity in said single cells.

29. The method of claim 28, wherein said tissue is a fresh tissue.

30. The method of claim 28, wherein said tissue is a frozen tissue.

31. The method of claim 28, wherein said tissue is a brain tissue.

32. The method of claim 28, wherein said tissue is a tumor.

33. The method of claim 28, wherein said tissue is a fertilized oocyte.

34. The method of claim 28, wherein said tissue is a biopsy.

35. The method of claim 28, wherein said isolating genomic DNA from said single cells comprises isolating genomic DNA from nuclei of said single cells.

36. A kit for measuring Line-1 retrotransposition activity in a single cell, said kit comprising Line-1 specific primers, control primers and a single cell.

37. A kit for measuring Line-1 retrotransposition activity in a tissue, said kit comprising Line-1 specific primers, control primers and a tissue.


SEQUENCE LISTING (190 sequences)

SEQ ID NO: 1 - DNA, 22 bp, Artificial Sequence
  PCR primer
aacaaagtaacactagagataa

SEQ ID NO: 2 - DNA, 20 bp, Artificial Sequence
  Pcr primer
tttggtgggagaattgggct

SEQ ID NO: 3 - DNA, 19 bp, Artificial Sequence
  PCR primer
ctggcgaggatgtggagaa

SEQ ID NO: 4 - DNA, 20 bp, Artificial Sequence
  PCR primer
cctgcaatcccaccaacaat

SEQ ID NO: 5 - DNA, 22 bp, Artificial Sequence
  PCR primer
taagagagcttgccagcagaga

SEQ ID NO: 6 - DNA, 22 bp, Artificial Sequence
  PCR primer
gcagacctgggagacagattct

SEQ ID NO: 7 - DNA, 22 bp, Artificial Sequence
  PCR primer
agagagcttgccagcagagagt

SEQ ID NO: 8 - DNA, 22 bp, Artificial Sequence
  PCR primer
gcagacctgggagacagattct

SEQ ID NO: 9 - DNA, 23 bp, Artificial Sequence
  PCR primer
tgtctcccaggtctgctgataga

SEQ ID NO: 10 - DNA, 27 bp, Artificial Sequence
  PCR primer
gattgttcttctggtgattctgttacc

SEQ ID NO: 11 - DNA, 21 bp, Artificial Sequence
  probe
ctgtaaactagttcaaccatt

SEQ ID NO: 12 - DNA, 23 bp, Artificial Sequence
  PCR primer
tgcggagaaataggaacactttt

SEQ ID NO: 13 - DNA, 21 bp, Artificial Sequence
  PCR primer
tgaggaatcgccacactgact

SEQ ID NO: 14 - DNA, 15 bp, Artificial Sequence
  PCR primer
aggtgggaattgaac

SEQ ID NO: 15 - DNA, 23 bp, Artificial Sequence
  PCR primer
caaacaccgcatattctcactca

SEQ ID NO: 16 - DNA, 23 bp, Artificial Sequence
  PCR primer
cttcctgtgtccatgtgatctca

SEQ ID NO: 17 - DNA, 16 bp, Artificial Sequence
  probe
aaggcttcagacgatc

SEQ ID NO: 18 - DNA, 25 bp, Artificial Sequence
  PCR primer
gaatgattttgacgagctgagagaa

SEQ ID NO: 19 - DNA, 24 bp, Artificial Sequence
  PCR primer
gtcctcccgtagctcagagtaatt

SEQ ID NO: 20 - DNA, 14 bp, Artificial Sequence
  probe
tcccagcacgcagc

SEQ ID NO: 21 - DNA, 24 bp, Artificial Sequence
  PCR primer
acagctttgaagagagcagtggtt

SEQ ID NO: 22 - DNA, 21 bp, Artificial Sequence
  PCR primer
agtctgcccgttctcagatct

SEQ ID NO: 23 - DNA, 18 bp, Artificial Sequence
  probe
tcttcgtttcaaaactag

SEQ ID NO: 24 - DNA, 22 bp, Artificial Sequence
  PCR primer
ggtcaatggcagaaaaggaaat

SEQ ID NO: 25 - DNA, 21 bp, Artificial Sequence
  PCR primer
cgcagtttgtgggaatgattc

SEQ ID NO: 26 - DNA, 14 bp, Artificial Sequence
  probe
agggtcgggcctgg

SEQ ID NO: 27 - DNA, 23 bp, Artificial Sequence
  PCR primer
ctcgtctgatctcggaagctaag

SEQ ID NO: 28 - DNA, 20 bp, Artificial Sequence
  PCR primer
gcggtctcccatccaagtac

SEQ ID NO: 29 - DNA, 16 bp, Artificial Sequence
  probe
cccttcgctgactctc

SEQ ID NO: 30 - DNA, 19 bp, Artificial Sequence
  PCR primer
aatggccccacccctatct

SEQ ID NO: 31 - DNA, 17 bp, Artificial Sequence
  PCR primer
gcgggctgagtccgaaa

SEQ ID NO: 32 - DNA, 24 bp, Artificial Sequence
  PCR primer
gctggatatgaaattctgggttga

SEQ ID NO: 33 - DNA, 24 bp, Artificial Sequence
  PCR primer
gctggatatgaaattctgggttga

SEQ ID NO: 34 - DNA, 21 bp, Artificial Sequence
  PCR primer
aaggggttagggagttttttt

SEQ ID NO: 35 - DNA, 22 bp, Artificial Sequence
  PCR primer
tatctataccctacccccaaaa

SEQ ID NO: 36 - DNA, 23 bp, Artificial Sequence
  PCR primer
agatcaaactgcaaggcggcaac

SEQ ID NO: 37 - DNA, 26 bp, Artificial Sequence
  PCR primer
tcttcaaagctgtcagacagggacac

SEQ ID NO: 38 - DNA, 28 bp, Artificial Sequence
  PCR primer
aataggaacagctccggtctacagctcc

SEQ ID NO: 39 - DNA, 27 bp, Artificial Sequence
  PCR primer
cgccgtttcttaagccggtctgaaaag

SEQ ID NO: 40 - DNA, 21 bp, Artificial Sequence
  PCR primer
gctgtccatcactgtccttca

SEQ ID NO: 41 - DNA, 21 bp, Artificial Sequence
  PCR primer
ccatctctgaagactacagcg

SEQ ID NO: 42 - DNA, 21 bp, Artificial Sequence
  probe
ctgtaaactagttcaaccatt

SEQ ID NO: 43 - DNA, 23 bp, Artificial Sequence
  PCR primer
tgcggagaaataggaacactttt

SEQ ID NO: 44 - DNA, 20 bp, Artificial Sequence
  PCR primer
cctccattgttggtgggatt

SEQ ID NO: 45 - DNA, 20 bp, Artificial Sequence
  PCR primer
aaccgccagactgatttcca

SEQ ID NO: 46 - DNA, 24 bp, Artificial Sequence
  PCR primer
gagcactgaaactcagaggagaga

SEQ ID NO: 47 - DNA, 25 bp, Artificial Sequence
  PCR primer
ctggtgattctgttaccgtctatca

SEQ ID NO: 48 - DNA, 16 bp, Artificial Sequence
  probe
ctgtctcccaggtctg

SEQ ID NO: 49 - DNA, 23 bp, Artificial Sequence
  PCR primer
ctcgtctgatctcggaagctaag

SEQ ID NO: 50 - DNA, 20 bp, Artificial Sequence
  PCR primer
gcggtctcccatccaagtac

SEQ ID NO: 51 - DNA, 14 bp, Artificial Sequence
  probe
agggtcgggcctgg

SEQ ID NO: 52 - DNA, 23 bp, Artificial Sequence
  PCR primer
tgcggagaaataggaacactttt

SEQ ID NO: 53 - DNA, 21 bp, Artificial Sequence
  PCR primer
tgaggaatcgccacactgact

SEQ ID NO: 54 - DNA, 23 bp, Artificial Sequence
  PCR primer
caaacaccgcatattctcactca

SEQ ID NO: 55 - DNA, 23 bp, Artificial Sequence
  PCR primer
cttcctgtgtccatgtgatctca

SEQ ID NO: 56 - DNA, 15 bp, Artificial Sequence
  probe
aggtgggaattgaac

SEQ ID NO: 57 - DNA, 25 bp, Artificial Sequence
  PCR primer
gaatgattttgacgagctgagagaa

SEQ ID NO: 58 - DNA, 24 bp, Artificial Sequence
  PCR primer
gtcctcccgtagctcagagtaatt

SEQ ID NO: 59 - DNA, 16 bp, Artificial Sequence
  probe
aaggcttcagacgatc

SEQ ID NO: 60 - DNA, 24 bp, Artificial Sequence
  PCR primer
acagctttgaagagagcagtggtt

SEQ ID NO: 61 - DNA, 21 bp, Artificial Sequence
  PCR primer
agtctgcccgttctcagatct

SEQ ID NO: 62 - DNA, 14 bp, Artificial Sequence
  probe
tcccagcacgcagc

SEQ ID NO: 63 - DNA, 22 bp, Artificial Sequence
  PCR primer
ggtcaatggcagaaaaggaaat

SEQ ID NO: 64 - DNA, 21 bp, Artificial Sequence
  PCR primer
cgcagtttgtgggaatgattc

SEQ ID NO: 65 - DNA, 18 bp, Artificial Sequence
  probe
tcttcgtttcaaaactag

SEQ ID NO: 66 - DNA, 19 bp, Artificial Sequence
  PCR primer
aatggccccacccctatct

SEQ ID NO: 67 - DNA, 17 bp, Artificial Sequence
  PCR primer
gcgggctgagtccgaaa

SEQ ID NO: 68 - DNA, 16 bp, Artificial Sequence
  probe
cccttcgctgactctc

SEQ ID NO: 69 - DNA, 85 bp, Homo sapiens
tgaggaatcgccacactgacttccacaatggttgagctagtttacagtcccaccaacagtgtaaaagtgttcctatttctccgca

SEQ ID NO: 70 - DNA, 85 bp, Homo sapiens
tgaggaatcgccacactgacttccacaatggttgaactagtttacagtcccaccaacagtgtaaaagtgttcctatttctccgca

SEQ ID NO: 71 - DNA, 85 bp, Homo sapiens; identical to SEQ ID NO: 70

SEQ ID NO: 72 - DNA, 85 bp, Homo sapiens; identical to SEQ ID NO: 70

SEQ ID NO: 73 - DNA, 85 bp, Homo sapiens; identical to SEQ ID NO: 70

SEQ ID NO: 74 - DNA, 85 bp, Homo sapiens
tgaggaatcgccacactgacttccaccatggttgaactagtttacagtcccaccaacagtgtaaaagtgttcctatttctccgca

SEQ ID NO: 75 - DNA, 85 bp, Homo sapiens
tgaggaatcgccacactgacttccacaatggttgaactagtttacagtcccatcaacagtgtaaaagtgttcctatttctccgca

SEQ ID NO: 76 - DNA, 85 bp, Homo sapiens; identical to SEQ ID NO: 70

SEQ ID NO: 77 - DNA, 85 bp, Homo sapiens
tgaggaatcgccacactgacttccacaatggttgaactagtttacagttccaccaacagtgtaaaagtgttcctatttctccgca

SEQ ID NO: 78 - DNA, 85 bp, Homo sapiens; identical to SEQ ID NO: 70

SEQ ID NO: 79 - DNA, 85 bp, Homo sapiens; identical to SEQ ID NO: 70

SEQ ID NO: 80 - DNA, 85 bp, Homo sapiens; identical to SEQ ID NO: 70

SEQ ID NO: 81 - DNA, 60 bp, Homo sapiens
tcaaacaccgcatattctcactcagaggtgggaattgaacaatgagatcacatggacaca

SEQ ID NO: 82 - DNA, 60 bp, Homo sapiens
tcaaacaccgcatattctcactcataggtgggaattgagcaatgagatcacatggacaca

SEQ ID NO: 83 - DNA, 61 bp, Homo sapiens
tcaaacaccgcatattctcactcataggtgggaactggacgatgagatcacatggacacag

SEQ ID NO: 84 - DNA, 60 bp, Homo sapiens
tcaaacaccgcatattctcactcataggtgggaattgaacaatgagatcacatggacaca

SEQ ID NO: 85 - DNA, 60 bp, Homo sapiens; identical to SEQ ID NO: 84

SEQ ID NO: 86 - DNA, 61 bp, Homo sapiens
tcaaacaccgcatattctcactcataggtgggaattgaacaatgagatcacatggacacag

SEQ ID NO: 87 - DNA, 60 bp, Homo sapiens; identical to SEQ ID NO: 84

SEQ ID NO: 88 - DNA, 61 bp, Homo sapiens; identical to SEQ ID NO: 86

SEQ ID NO: 89 - DNA, 59 bp, Homo sapiens
tcaacaccgcatattctcactcataggtgggaattgaacaatgagatcacatggacaca

SEQ ID NO: 90 - DNA, 59 bp, Homo sapiens; identical to SEQ ID NO: 89

SEQ ID NO: 91 - DNA, 61 bp, Homo sapiens; identical to SEQ ID NO: 86

SEQ ID NO: 92 - DNA, 59 bp, Homo sapiens
tcaaaccgcatattctcactcataggtggggattgaacaatgagatcacatggacacag

SEQ ID NO: 93 - DNA, 59 bp, Homo sapiens; identical to SEQ ID NO: 89

SEQ ID NO: 94 - DNA, 61 bp, Homo sapiens; identical to SEQ ID NO: 86

SEQ ID NO: 95 - DNA, 59 bp, Homo sapiens
tcaacaccgcatattctcactcataggtgggaattgaacaatgaatcacatggacacag

SEQ ID NO: 96 - DNA, 61 bp, Homo sapiens; identical to SEQ ID NO: 86

SEQ ID NO: 97 - DNA, 85 bp, Homo sapiens; identical to SEQ ID NO: 70

SEQ ID NO: 98 - DNA, 85 bp, Homo sapiens; identical to SEQ ID NO: 70

SEQ ID NO: 99 - DNA, 85 bp, Homo sapiens
tgaggaatcgccacactgacttccacaatggttgaactagtttacagtcccatgaactgtgtaaaagtgttcctatttctccgca

SEQ ID NO: 100 - DNA, 85 bp, Homo sapiens; identical to SEQ ID NO: 70

SEQ ID NO: 101 - DNA, 79 bp, Homo sapiens
tgaggaatcgccacactgacttccacaatggttgaactagtttacagtcccaccaacagtgtaaaagtgttcctatttc

SEQ ID NO: 102 - DNA, 85 bp, Homo sapiens
  misc_feature (10)..(10): n is a, c, g, or t
tgaggaatcnccacactgacttccacaatggttgaactagtttacagtcccaccaacagtgtaaaagtgttcctatttctccgca

SEQ ID NO: 103 - DNA, 85 bp, Homo sapiens; identical to SEQ ID NO: 70

SEQ ID NO: 104 - DNA, 83 bp, Homo sapiens
  misc_feature (31)..(31): n is a, c, g, or t
tgaggaatcgccacactgacttccacaatgnttgaactagtttacagtcccaccaacagggtaaaagtgttcctatttctccg

SEQ ID NO: 105 - DNA, 81 bp, Homo sapiens
tgaggaatcgccacactgacttccacaatggttgaactagtttacagtcccaccaaccgtgtaaaagtgttcctatttctc

SEQ ID NO: 106 - DNA, 85 bp, Homo sapiens; identical to SEQ ID NO: 70

SEQ ID NO: 107 - DNA, 83 bp, Homo sapiens
tgaggaatcgccacactgacttccacaatggttgaactagttgacagtcccaccaacagtgtaaaagtgttcctatttctccg

SEQ ID NO: 108 - DNA, 85 bp, Homo sapiens; identical to SEQ ID NO: 70

SEQ ID NO: 109 - DNA, 85 bp, Homo sapiens
tgaggaatcgccacactgacttccacaatggttgaactagttgacagtcccaccaacagtgtaaaagtgttcctatttctccgca

SEQ ID NO: 110 - DNA, 85 bp, Homo sapiens; identical to SEQ ID NO: 70

SEQ ID NO: 111 - DNA, 85 bp, Homo sapiens; identical to SEQ ID NO: 70

SEQ ID NO: 112 - DNA, 85 bp, Homo sapiens; identical to SEQ ID NO: 70

SEQ ID NO: 113 - DNA, 85 bp, Homo sapiens; identical to SEQ ID NO: 70

SEQ ID NO: 114 - DNA, 85 bp, Homo sapiens; identical to SEQ ID NO: 70

SEQ ID NO: 115 - DNA, 85 bp, Homo sapiens; identical to SEQ ID NO: 70

SEQ ID NO: 116 - DNA, 85 bp, Homo sapiens; identical to SEQ ID NO: 109

SEQ ID NO: 117 - DNA, 83 bp, Homo sapiens; identical to SEQ ID NO: 107

SEQ ID NO: 118 - DNA, 85 bp, Homo sapiens; identical to SEQ ID NO: 70

SEQ ID NO: 119 - DNA, 85 bp, Homo sapiens; identical to SEQ ID NO: 70

SEQ ID NO: 120 - DNA, 85 bp, Homo sapiens; identical to SEQ ID NO: 70

SEQ ID NO: 121 - DNA, 83 bp, Homo sapiens
tgaggaatcgccacactgacttccacaatggttgaactagtttacagtcccaccaacagtgtaaaagtgttcctatttctccg

SEQ ID NO: 122 - DNA, 85 bp, Homo sapiens; identical to SEQ ID NO: 70

SEQ ID NO: 123 - DNA, 85 bp, Homo sapiens; identical to SEQ ID NO: 70

SEQ ID NO: 124 - DNA, 909 bp, Homo sapiens
gggggaggagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttctgagtcaaagaaaggggtgacggtcgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcagccgggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagacagactgcctcctcaagtgggtccctgactcctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaccacaaaga

SEQ ID NO: 125 - DNA, 910 bp, Homo sapiens
cccgcagttaacgcgactagtaccggcaatagaggacaggcctatgattagactgcttaggcccaggggactatggaagcccgcccaggggcacgcctactgcaagtgagatggtaggttcctggtgtgcgatccagagtctggccgttgcttgctgatggactgtacgctgacgaacgagggaacggttttggggagttgtggggcagcggtgggagctccacgccggaacgaaaccaagccctggaggaatcgttaattgcggtcccgtaccgaagcttggtacgacctgtacggaggccaacaatcccgcctggccacgcaaagcttgccccgtcgatacacgtgcacagcgcagtaaccggcgagaagaggctcccggcactgaacacaacaggaaaatgcgccaggttacgacgagcttagtggaaagaggcctagcctctcagctgaggcgcttgaaccgtacatttaccgggacgggctgcaccaagccgcgaatgatggcgtcacgaccgagaatcggtccacgataactagcacatgccatcacatctgccaatgtcgatccggaagcttccaaggaacgccccatggcctagtcaaggtatgcctggtgctaatagggcccatgagatcactaatctaccgcccagaaacatggccgccatcacacaacggaggacgagcggtctagaaagcctgttaagactaagggccagcgaaccggctaacgtaaggcaggcccttccaccctctgcgcggtacctccacccgattgaatgcccatgcatacgtgaacgcccactttaaagagcaccgcctatgctacgacgataaaacaaaggcatccagaggtcaagaaaaaggataacagcgagaggcagctcagcaggccccgagcgaacgc

SEQ ID NO: 126 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagagaaagtcgggttacccacaaagggaagcccatcagactaacagtggatctctcggcagaaaccctacaagccagaagagagtgggggccaatattcaacattcttaaagaaaagaattttcaacccagaattcatatccagc

SEQ ID NO: 127 - DNA, 257 bp, Homo sapiens
aggaaatacagagaacgccacaaagatactcctcgagaagagcaactccatgacacataattgtcagattcaccaaagttgaaatgaaggaaaaaatgttaagggcagccagagagaaagtcgggttaccctcaaaggaaagcccatcagactaacagcggatctctcggcagaaaccctacaagccagaagagagtgggggccaatattcaacattcttaaagaaaagaattttcaacccagaattcatatccagc

SEQ ID NO: 128 - DNA, 257 bp, Homo sapiens
aggaaatacagagaacgccacaaagatgctcctcgagaagagcaactccaagacacataattgtcagattcaccaaagttgaaatgaaggaaaaaatgttaagggcagccagagagaaagtcgggttactcacaaagggaagcccatcagactgacagcggatctctcagcagaaactctacaagccagaagagagtgggggccaatattcaaaattcttaaagaaaagaattttcaacccagaattcatatccagc

SEQ ID NO: 129 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagagaaagtcgggttacccacaaagggaagcccatcagactaacagcagatctcttggcagaaaccctacaagccagaagagagtgggggccaatattcaacattcttaaagaaaagaattttcaacccagaattcatatccagc

SEQ ID NO: 130 - DNA, 257 bp, Homo sapiens
aggaaatacagagaacgccacaaagatactcctcaagaagagcaactccaagacacataactgtcagattcaccaaagttgaaatgaaggaaaaaatgttaagggcagccagagagaaagtcaggttaccctcaaaggaaagcccatcagactaacagcggatctctcggcagaaaccctacaagccagaagagagtgggggccaatattcaacattcttaaagacaagaattttcaacccagaattcatatccagc

SEQ ID NO: 131 - DNA, 257 bp, Homo sapiens
aggaaatacagagaacgccacaaagatactcctcgagaagagcaactccaagacacataattgtcagattcaccgaagttgaaatgaaggaaaaaatgttaagggcagccagagagaaagtcgggttaccctcaaagggaagcccatcagactaacagcggatctctcggcagaaaccctacaagccagaagagaggggggccaatattcaacattcttaaagaaaagaattttcaacccagaatttcatatccagc

SEQ ID NO: 132 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagagaaagtcgggttaccctcaaagggaagcccctcagactaacagctgatctctcagcagaaactctacgagccagaagagagtgggggccaatattcaacattcttaaagaaaagaattttcaacccagaattcatatccagc

SEQ ID NO: 133 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagaaaaggtcaggttacccacaaagggaagcccatcagactaacagtggatctctctgcagaaactctacaagccagaagagagtgggggccaatatccaacattcttaaagaaaagaattttcaacccaaatttcatatccagc

SEQ ID NO: 134 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagagaaagtcgggttacccacaaagggaagcccatcagactaacagcggatctctcggcacaaactctacaagccagaagagaatgggagccaatattaaacattcttaaagaaaagaattttcaacccagaattcatatccagc

SEQ ID NO: 135 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagagaaagtcggattactcacaaagggaagcccatcagactaacagctgatctctcagcagaaactctacaagccagaagagagtgggggccaatattcaacattcttaaagaaaagaattttcaacccagaattcatatccagc

SEQ ID NO: 136 - DNA, 257 bp, Homo sapiens
aggaaatacagagaacgccacaaagacactcctcgagaagagcaactccaagacacataattgtcagattcaccaaagttgaaatgaaggaaaaaatgttaagggcagccagagagaaagtcgggttacccacaaagagaagaccatcagactaacagcagatctcttggcagaaactctacaagccagaagagagtgggggccaatattcaacattcttaaagaaaaggattttcaacccagaattcatatccagc

SEQ ID NO: 137 - DNA, 257 bp, Homo sapiens
aggaaatacagagaacgccacaaagatactcttcgagaagagcaaccccaagacacgtaattgtcagattcaccaaagttgaaatgaaggaaaaaatgttaagggcagccagagagaaagtcgggttaccctcaaagggaagcccatcagactaacagcagatctctctgcagaaactctacaagccagaagagagtgggggccaatattcaacattcttaaagaaaagaattttcaacccagaattcatatccagc

SEQ ID NO: 138 - DNA, 257 bp, Homo sapiens
aggaaatacagagaacgccacaaagatactcctcgagaagagcaaccccaagacacataattgtcagattcaccaaagttgaaacgaaggaaaaaatgttaagggcagccagagagaaagttgggttacccacaaagggaagcccatcagactaacagcagatctcttggcagaaaccctacaagccagaagagagtgggggccaatattcaacattcttaaagaaaaggattttcaacccagaattcatatccagc

SEQ ID NO: 139 - DNA, 257 bp, Homo sapiens
aggaaatacagagaacgccacaaagatactccttgagaagaccaactccaaaacacctaattgtcaaattcaccaaagttgaaatgaaggaaaaaatgttaagggcagccagagagaaaatcggcttacccacaaagggaagcccatcagactaacagctgatctcttggcagaaattctacaagccagaagagagtgggggccaatattcaacattcttaaagaaaagaattttcaacccagaattcatatccagc

SEQ ID NO: 140 - DNA, 257 bp, Homo sapiens
aggaaatacagagaacgccacaaagatactcttcgagaagagcaaccccaagacacataattgtcagatttgccaaggttgaaatgaaggaaaaaatgttaagggcagccagagagaaagtcgggttacccacaaagggaagcccatcagactaacagcggatctctcagcagaaactctataagccagaaaagagtgggggccaatattcaacattgttaaagaaaataattttcaacccagaattcatatccagc

SEQ ID NO: 141 - DNA, 257 bp, Homo sapiens
aggaaatacagagaacgccacaaagatactcctcaagaagagcaactccaagacacgtaattgtcagattcaccaaagttgaaatgaaggaaaaaatgttcagggcagccagagagaaagttgggttacccacaaagggaagcctatcagactaactgctgatgtctcagcagaaactctacgagccagcagagagtgggggccaatattcaacattcttaaagaaaagaattttcaacccagaattcatatccagc

SEQ ID NO: 142 - DNA, 258 bp, Homo sapiens
aggaaatacagagaacgccacaaagatactcctcgagaagggcaaccccaagacacataatcatcagattcaccaaggttgaaatgaaggaaaaatgttaagggcagccagagagaaaggttgggttacccacaaagggaagcccatcagacttaacaggggatctttctgtagaaatcctacaagccagaagagagtgggggccaatattcaacattcttaagaaaagaattttcaacccagaatttcatatccagc

SEQ ID NO: 143 - DNA, 257 bp, Homo sapiens
aggaaatacagagaacgccacaaagatactcctcgagaagagcaactccaagacacataattgtcagattcaccaaagttgaactgaaggaaaaaatattaagggcagccagagagaatgtcgggttacccacaaagggaaggctatcagactaacagcagatctcttggcagaaattctacaagccagaagagagtgggggccaatattcaacaatcttaaagaaaagaattttcaacccagaattcatatccagc

SEQ ID NO: 144 - DNA, 257 bp, Homo sapiens
aggaaatacagagaacgccacaaagatactcctccagaagagcaaccccaagacacatcattgtcagattcaccaaggttgaaatgaaggaaaaagtgttaagggcagccagagagaaagttgagttacccacagagggaagcccatcagactaacagcggatctctcagcagaaaccctacaagccagaagaaagtgggagccaatagtcaacattcttaaaggaaagaattttcaacccagaattcatatccagc

SEQ ID NO: 145 - DNA, 257 bp, Homo sapiens; identical to SEQ ID NO: 144

SEQ ID NO: 146 - DNA, 257 bp, Homo sapiens
aggaaatacagagaacgccacaaagatgctctttgagaagagtaaccccaagacacataaccatcagattcaccaaggttgaaatgaaggaaaaaatgttacgggcaaccagagagaaagctgggttacccacaaatggaagcccatcagactacagtggatatctctgcagaaaccctacaagccagaaaagagtgggggccaatattcaacattcttaaagaaaagaattttcaacccagaatttcatatccagc

SEQ ID NO: 147 - DNA, 259 bp, Homo sapiens
aggaaatacagagaacgccacaaaaatactccttgagaagagcaaccccaaggcacataatcctcagattcaccaacgttgaaatgaaggaaaaaaatgttgagagcagccagagagaaagtcgggttacccacaaagagaagcccatcagattaaccatgaatctctctgcagaaaccctacaagccagaagagagtgggagccaattattcaacattcttaaagaaaataattttcaacccagaattcatatccagc

SEQ ID NO: 148 - DNA, 257 bp, Homo sapiens
aggaaatacagagaacgccacaaagatacacctcgagaagagcaaccccaggacacatggttgtcagattcaccaaggttgaaatgaaggaaagaatgttgagggcggccagagagaaagtcgggttgcccacgaagggaggtccatcggactaacagcggatctctctgcagagaccctgcaagccagaagagagtgggggccagtattcaacattcttaaagaaaagaattttcaacccagaattcatatccagc

SEQ ID NO: 149 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagagaaaggcgggttaccctcaaaggaaagcccatcagactaacagcggatctctcggcagaaaccctacaagccagaagagagtgggggccaatattcaacattcttaaagaaaagaattttcaacccagaatttatatccagc

SEQ ID NO: 150 - DNA, 257 bp, Homo sapiens
aggaaatacagagaacgccacaaagatactcctcaagaagagcaactccaagacacataattgtcagattcaccaaagttgaaatgaaggaaaaaatgttaagggcagccagagagaaaggcgggttaccctcaaagggaagcccatcagactaacagcggatctcttggcagaaaccctacaagccagaagagagtgggggccaatattcaacattcttaaagaaaagaattttcaacccagaatttatatccagc

SEQ ID NO: 151 - DNA, 257 bp, Homo sapiens
  misc_feature (10)..(10): n is a, c, g, or t
aggaaatacngagaacgccacaaacatactcctcgagaagagcaactccaagacacataattgtcagattcaccaaagttgaaatgaaggaaaaaatgttaagggcagccagagagaaaggcgggttacccacaaagggaagcccatcagactaacagcggatctctcggcagaaaccctacaagccagaagagggtgggggccaatattcaacattcttaaagaaaagaattttcaacccagaatttatatccagc

SEQ ID NO: 152 - DNA, 257 bp, Homo sapiens
gctggatatgaaattctgggttgaaaattcttttctttaagaatgttgaatattggcccccactctcttctggcttgtagggttttggccgagagatcagctgttagtctgatgggcttcctttgtgggtaacccaacctttctctctggctgcccttaacattttttccttcatttcaactttggtgaatctgacaattatgtgtcttggagttgctcttctccaggagtatctttgtggcgttctcgtatttcct

SEQ ID NO: 153 - DNA, 257 bp, Homo sapiens
aggaaatacagagaacgccacaaaaatactcctcgagaagagcaactccaagacacataattgtcagattcaccaaagttgaaatgaaggaaaaaatgttaagggcagccagagagacatgcgggttaccctcaaagggaagcccatcacactaacagcggatctctctgcagaaacgctacaagccagaagagagtgggggccaatattcaacattcttaaagaaaagaattttcaacccagaatttatatccagc

SEQ ID NO: 154 - DNA, 257 bp, Homo sapiens
gctggatatgaaattctgggttgaaaattcttttctataagaacgttgaatattggcccccactctctcctggcttgtagggtttctgccaagagatccactgttagtctgatgagcttcctttgtaggtaacccaacctttctctctggctgcccttaacattttttccttcatttcaaccttggtgaatctgacaattatgtgtcttggggttgctcttctcaaggagtatctttgtggcgttctcgtatttcct

SEQ ID NO: 155 - DNA, 257 bp, Homo sapiens
aggaaatacagagaacgccacaaagatactcctcgagaagaggaatcccaagacacataatcatcagatccaccaaggttgaaatgaaggaaaaaatgttaagggcagccagagagaaaggcgggttacccacaaagggaagcccatcagactaacagtggatctctcgggagaaactctacaagccagaagagagtgggggccaatattcgacattcttaaagaaaagaattttcaacccagaatttatatccagc

SEQ ID NO: 156 - DNA, 257 bp, Homo sapiens; identical to SEQ ID NO: 154

SEQ ID NO: 157 - DNA, 257 bp, Homo sapiens; identical to SEQ ID NO: 155

SEQ ID NO: 158 - DNA, 257 bp, Homo sapiens; identical to SEQ ID NO: 154

SEQ ID NO: 159 - DNA, 257 bp, Homo sapiens; identical to SEQ ID NO: 154

SEQ ID NO: 160 - DNA, 257 bp, Homo sapiens; identical to SEQ ID NO: 154

SEQ ID NO: 161 - DNA, 257 bp, Homo sapiens
aggaaatacagagaacgccacaaagatactcctcgagaagagcaactccaagatacataaaggaaatacagagaacgccacaaagatactcctcgagaagagcaactccaagatacataagtggattacccacaaagggaagcccatcagactaacagctgatctctcggcagaaactctataagccagaagagtggggggccaatattcaacattcttaaagaaaagaattttcaacccagaatttcatatccagc

SEQ ID NO: 162 - DNA, 260 bp, Homo sapiens
gctggatatgaaattctgggttgaaaattcttttctttcagaatgttgaatattggaccccactctcttctggcttgtagagtttctgccgagagatccactgttaagtctgatgggcttcctttgcaggtaacctgacctttctctctggctgcccttaatatgttttccttcatttcaactttggtgaatctgacaatttatgtgtcttggagttgctcttctcgaggtgttacctttgtggcgtttctgtatttcct

SEQ ID NO: 163 - DNA, 261 bp, Homo sapiens
  misc_feature (3)..(3): n is a, c, g, or t
scnsaggaaatacagagaacgccacaaagatactcctcgagaagagcaactccaagatacataattgtcagattcaccaaagttgaaatgaagaaaaaatgttaagggccgccagagagaaaggttggattacccacaaagggaagcccatcagactaacagctgatctctcggcagaaactctataagccagaagagtggggggccaatattcaacattcttaaagaaaagaattttcaacccagaatttcatatccagc

SEQ ID NO: 164 - DNA, 257 bp, Homo sapiens
aggaaatacagagaacgccacaaagatactcctcaagaagagcaaccccaagacgcataattgtcagattcaccaaagttgaaagaaggaaaaaatgttaagggcagccagagagaaaggtcaggttgcccacaaagggaagcccatcggactaacagtggatccctaagcagaaactctacaagccagaagacagtgggggccaatattcacattcttaaagaaaagaattttcaacccagaatttcatatccagc

SEQ ID NO: 165 - DNA, 255 bp, Homo sapiens
aggaaatacagagaacgccacaaagatattcctcgagaagagcaaccccaagacacataatcatcagattaaccaaggttgaaatgaaggaaaaaatgttaagggcagccagagagaaaggcgggttacccacaaagggaagcccatcagactaacagcagatctcttggaagaaaccctataagccagaagacagtgggggccaatattcaacattcttaaagagaattttcaacccagaatttcatatccagc

SEQ ID NO: 166 - DNA, 257 bp, Homo sapiens
gctggatatgaaattctgggttgaaaattcttttctttaagaatgttgaatattagcccccaatctcttgtggcttttagggtttctgcagagagatctgctgttagtatgatgggcttcctttgtaggtaacccaacttttctctctggctgcccttaacattttttctttcagttcaaccttggtgaatctgacgattatgtgtcttggggttgctcttctcaaagagtatctttgtggcgttctcgtatttcct

SEQ ID NO: 167 - DNA, 257 bp, Homo sapiens
aggaaatacagagaacgccacaaagatactcctcgagaagagcaaccccaagacacataatcatcagattcaccaaggttgaaatgaaggaaaaaatgttaagggcagccagagagaaaggcacattacccacaaagggaaacccatcagactaacggcggatctctctgcagaaacgctacaacccagaaaagagtgggggcacacatttaacattcttaaggaaaagaattttcaacccagaatttatatccagc

SEQ ID NO: 168 - DNA, 249 bp, Homo sapiens
aggaaatacagagaacgccacaaagatactccttgagaagagcaaccccaagacacataattgtcagattcaccaaggttgaaatgaaggaaaaagtgttaagggcagccaaagagaaaggttagttacccacaaagggacgcccatcagactaacagtggatctctctgcagaaaccctacaagaagagagtgggggccaatattcaacattcttaaagaattttcaacccagaatttcatatccagc

SEQ ID NO: 169 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagagaaaggtgggttaccctcaaaggaaagcccatcagactaacagcggatctctcggcagaaaccctacaagccagaagagagtgggggccaatattcaacattcttaaagaaaagaattttcaacccagaatttctatccagc

SEQ ID NO: 170 - DNA, 257 bp, Homo sapiens; identical to SEQ ID NO: 169

SEQ ID NO: 171 - DNA, 257 bp, Homo sapiens; identical to SEQ ID NO: 169

SEQ ID NO: 172 - DNA, 257 bp, Homo sapiens
aggaaatacagagaacgccacaaagatactcctcgagaagagcaactccaagacacataaatgtcagattcaccaaagttgaaatgaaggaaaaaatgttaagggcagccagagagaaaggtgggttaccctcaaagggaagcccatcagactaacagcggatctctcggcagaaaccctacaagccagaagagagtgggggccaatattcaacattcttaaagaaaagaattttcaacccagaatttctatccagc

SEQ ID NO: 173 - DNA, 257 bp, Homo sapiens
aggaaatacagagaacgccacaaagatactcctcgagaagagcaactccaagacacataattgtcagattcaccaaagttgaaatgaaggaaaaaatgttaagggcagccagagagaaaggtgggttaccctcaaagggaagcccatcagactaacagcggatctcttggcagaaactctacaagccagaagatagtgggggccaatattcaacattcttaaagaaaagaattttcaacccagaatttctatccagc

SEQ ID NO: 174 - DNA, 257 bp, Homo sapiens
  misc_feature (10)..(10): n is a, c, g, or t
aggaaatacnganaacgccacaaagatactcctcgagaagagcaactccaagacacataattgtcagattcaccaaagttgaaatgaaggaaaaaatgttaagggcagccagagagaaaggtgggttaccctcaaaggaaagcccatcagactaacagcggatctctcggcagaaaccctacaagccagaagagagtgggggccaatattcaacattcttaaagaaaagaattttcaacccagaatttctatccagc

SEQ ID NO: 175 - DNA, 257 bp, Homo sapiens
  misc_feature (10)..(10): n is a, c, g, or t
aggaaatacngagaacgccacaaagatactcctcgagaagagcaactccaagacacataattgtcagattcaccaaagttgaaatgaaggaaaaaatgttaagggcagccagagagaaaggtgggttaccctcaaagggaagcccatcagactaacaacggatctctcggcagaaaccctacaagccagaagagagtgggggccaatattcaacattcttaaagaaaagaattttcaacccagaatttctatccagc

SEQ ID NO: 176 - DNA, 256 bp, Homo sapiens
aggaaatacagagaacgccacaaagatactcctcgagaagagcaactccaagacacataattgtcagattcaccaaagttgaaatgaaggaaaaaatgttaagggcagccagagagaaaggtgggttactctcaaagggaagcccatcagactaacagcggatctctcggcagaaaccctacaagccagaagagagtgggggccaatattcaacattcttaaagaaaagaattttcaaccagaatttcaatccagc

SEQ ID NO: 177 - DNA, 255 bp, Homo sapiens
  misc_feature (11)..(12): n is a, c, g, or t
gaaatactcannacgccacaaagatactcctcgagaagagcaactcaaagacacataattgtcagattcaccaaagttgaaatgaaggaaaaaatgttaagggcagccagagagaaaggtcggttaccctctaagggaagcccatcagactaacagcggatctctcggcagaaaccctacaaaccagaagagagtgggggccaatattcaacattcttaaagaaaagaattttcaacccagaatttcattccagc

SEQ ID NO: 178 - DNA, 257 bp, Homo sapiens
aggaaatacagagaacgccacaaagatactcctcgagaagggcaactgcaagacacataattgtcagattcaccaaagttgaaatgaaggaaaaaatgttaagggcagccagagagaaaggtgggttcccctcaaagggaagcccatcagactaacagcggttctctcggcagaaaccccacaagccagaagacagtggggaccaatattcaacattcttaaagaaaagaattttcaacccagaatttctatccagc

SEQ ID NO: 179 - DNA, 198 bp, Homo sapiens
aggaaatacagagaacgccacaaagatactcttcgagaagagcaaccccaagacacataattgtcagattcaccaaggttgaaaggaaggaaaaaatgttaagggcagccagagagaaagtaaagccagaagagagtgggggccaatattcaacattcttaatgaaaagaattttcaacccagaatttcatatccagc

SEQ ID NO: 180 - DNA, 257 bp, Homo sapiens
aggaaatacagagaacgccacaaagatactcctcgagaagagcaactccaagacacataatcgtcagattcaccaaggttgaaatgaaggaaaaaatgttaaggattaccagagagaaaggtgggttccccacaaaggaaagcccatcagactaacagcggatctctcggcagaaactctacaagccagaagagagtgggggccaatattcagcattcttaaagaaaagaattttcaacccagaatttctatccagc

SEQ ID NO: 181 - DNA, 257 bp, Homo sapiens
aggaaatacagagaacgccacaaagatactccatgagaagagtaaccccaagaaatataattgtcagattcaccaaggttgaaatgaaggaaaaaatgttaagggcagccagagagaaaggtgggttacccacaaagggaagcccatcagactaaaagcagatctctcggcagaaaccctataagccagaaaagagtgggggccaatattcaacattcttaaagaaatgaattttcaacccagaatttctatccagc

SEQ ID NO: 182 - DNA, 257 bp, Homo sapiens
aggaaatacagagaacgccacaaagatagtccttgagaagagcaactccaagacacataattgtcagattcaccaaagttgaaatgaagaaacaaatgttcagggcagccagagagaaaggtaggttacccacaaagggaagcccatcagactaacagctgatctctctgcagaaactctaccagccagaagagagtggggaccaatattcaacattcttaaagaaaagaattttcaacccagaatttctatccagc

SEQ ID NO: 183 - DNA, 257 bp, Homo sapiens; identical to SEQ ID NO: 181

SEQ ID NO: 184 - DNA, 257 bp, Homo sapiens; identical to SEQ ID NO: 181

SEQ ID NO: 185 - DNA, 256 bp, Homo sapiens
gctggatatgaaattctgggttgaaaattcttttctttaagaatgttgaatattggcccccactctcttctggtttgtagggtttctgcaaagagatccgctgttagtctgatgggcttctcttgtgggtaacccgacctttctctctggctgcccttaacattttttccttcatttcaaccttggtgaatctgacaattatttgtcttggggttgctgtctcgaagagtgtctttgtggcgttcttgtatttcct

SEQ ID NO: 186 - DNA, 256 bp, Homo sapiens
gctggatatgaaattctgggttgaaaattcttttctttaagaatgttgaatactggcccccactctcttctggattggagtttctgctgagagatcagttgttagtctgatgggcttccctttgtgggtaacccgacctttctctctggctgcccttaacattttctccttcatttcaactttggtgaatctgacaattatgtgtctggagttgctcttctcgaggagcatctttgtggcgttctctgtatttcct

SEQ ID NO: 187 - DNA, 256 bp, Homo sapiens
aggaaatacagagaacgccacaaagatactccctgcgaagagcaaccccaagacacataatcgtcagattcccaaggttgaaatgaaggaaaaaatgttaagggcagccaaagagaaaggttggattacccccaaagggacgcccattgaactaacagcagatctctctgcaaaaaccctacaagccagaatagagtggggccaatattcaacattcttaaagaaaagaatttcaacccagaatttcatatccagc

SEQ ID NO: 188 - DNA, 256 bp, Homo sapiens
aggaaatacagagaacgccacaaagatactccttgcgaagagcaaccccaagacacataatcgtcagattcccaaggttgaaatgaaggaaaaaatgttaagggcagccaaagagaaaggttggattacccccaaagggacgcccattgaactaacagcagatctctctgcaaaaaccctacaagccagaatagagtggggccaatattcaacattcttaaagaaaagaatttcaacccagaatttcatatccagc

SEQ ID NO: 189 - DNA, 80 bp, Homo sapiens
ttgaataacatagaatgaagatttagtgttaccgttaacaaacttattgtatcttacttctaaatcacaatggcaattgt

SEQ ID NO: 190 - DNA, 40 bp, Homo sapiens
  misc_feature (14)..(14): truncated L1 transposon inserted here
aataacatagaatgaagaagatttagtgttaccgttaaca